(19) United States
(12) Patent Application Publication
(10) Pub. No.: US 2024/0355474 A1
(43) Pub. Date: Oct. 24, 2024

(54) METHODS FOR DETERMINING THE BENEFITS AND RISKS IN HEALTHCARE APPLICATIONS

(71) Applicant: Richard George MATT, JR., Grayslake, IL (US)

(72) Inventor: Richard George MATT, JR., Grayslake, IL (US)

(21) Appl. No.: 18/643,355

(22) Filed: Apr. 23, 2024

Related U.S. Application Data
(60) Provisional application No. 63/461,562, filed on Apr. 24, 2023.

(51) Int. Cl. G16H 50/20; G16H 15/00

(52) U.S. Cl. CPC G16H 50/20; G16H 15/00

(57) ABSTRACT

Disclosed herein are novel methods and systems for defining, determining, and/or calculating or quantifying the benefits of one or more healthcare actions (e.g., products and/or services such as, for instance, medical devices and/or medical procedures). In particular, the application relates to methods and systems for determining (e.g., calculating and/or quantifying) whether the benefit of a healthcare action exceeds the benefit. In at least one example, the calculation of one or more benefits is achieved via the same scale used to calculate risks.

### CROSS-REFERENCE TO RELATED APPLICATION

This application claims priority to U.S. Provisional Application No. 63/461,562, filed Apr. 24, 2023, which is hereby incorporated by reference in its entirety.

### FIELD

The application relates generally to defining, determining, and/or calculating or quantifying the benefits of various situations in healthcare applications, including, for instance, performing one or more healthcare actions, as described below herein. In particular, the application relates to methods for determining (e.g., calculating and/or quantifying) whether the benefit of a healthcare action exceeds the risk. Such determination of benefit and risk uses the same scale, thereby permitting an accurate benefit-risk analysis.

### BACKGROUND

Risk management relates to the identification and evaluation of risks. Such risks may be due to various internal (e.g., issues in the design, production, and/or use of a product) and external (e.g., issues inherent to the options (even without any design, production, and/or use failures), and/or natural disasters) factors. Generally, risk management may involve, for instance, identifying risks, quantifying their potential severity, and/or quantifying their chance of occurring. An extension of risk management may also involve, for example, benefit-risk analysis.

Accordingly, risk management processes are used in various fields whenever a comparison of a given situation's risks and benefits is useful and/or necessary. These comparisons frequently guide significant, program-level decisions in a wide variety of industries, including, for instance, medicine, aviation, construction, defense, automotive, telecommunications, and the like.

As a further example, United States (U.S.) federal law requires that the likely benefit of a healthcare action outweighs that device's likely risk. See, e.g., Code of Federal Regulations (C.F.R.), Title 21, Section 820.30 (g). Although guidance based on this requirement sets forth specific requirements for assessing risk, there are a lack of methods for identifying and quantifying benefits. The same is true with respect to both healthcare action guidance documents and program-level decision processes in other fields.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure provides an objective way to identify, determine, calculate, and/or quantify whether the benefit of performing a healthcare action exceeds the risk of doing so. A “healthcare action” is defined further below herein and includes, for instance, following the instructions for use for a product and/or a service (e.g., a medical device, medical procedure, and the like). As used herein, the term “objective” does not necessarily imply that the aforementioned embodiments eliminate all uncertainty in benefit-risk analysis, but rather that such embodiments provide methods to quantify the benefit and risk in a defined manner that is significantly more repeatable than known methods.

In at least one example, the aforementioned calculation of one or more benefits is achieved via a measurement scale that may also be used to calculate risk of the same product and/or service. Measuring benefit with the same measurement scale used to calculate and/or quantify risk results in a straightforward process to compare benefits and risks, thereby simplifying the problem of determining whether benefits or risks of the given decision, product and/or service are greater. In at least another example, different metrics for benefit and risk could be defined, and a relationship could be established between these different metrics.

In at least one example, a method includes defining (e.g., by at least one processor) an appropriate risk metric (e.g., including possible risks to a patient's health).

In at least one example, a method includes defining (e.g., by at least one processor) one or more equations (e.g., Equation 9 as defined below herein) that relate benefit and risk. Risks can be defined (e.g., by using one or more tables) with reference to different levels of probability and severity.

In at least one example, a method includes utilizing (e.g., by at least one processor) one or more risk algebra rules for simplifying the aforementioned one or more equations, to determine whether the benefit of a healthcare action (e.g., a product and/or service, including a medical device, medical procedure, and the like) exceeds the risk.

In at least one example, a method includes creating (e.g., by at least one processor) and/or identifying one or more equations that define benefit and risk, based on the specific situation(s) to be reviewed and/or examined in a benefit-risk analysis.

In at least one example, a method includes selecting (e.g., by at least one processor) a best healthcare action (e.g., a best medical treatment and/or therapy) for a patient from a list of alternative healthcare actions (e.g., alternative medical treatments and/or therapies).

In at least one example, a method includes customizing (e.g., by at least one processor) a benefit-risk analysis for an individual (e.g., an individual patient) based on that individual's preferences.

In at least one example, a method includes modeling (e.g., by at least one processor) a risk that varies arbitrarily with time.

In at least one example, a method includes estimating (e.g., by at least one processor) the uncertainty associated with a benefit-risk analysis.

In at least one example, any one or more of the methods described herein may be executed, in whole or in part, on any of the computing devices and/or computing systems described herein. For instance, computing systems are described for identifying and/or calculating benefits of a healthcare action, and for comparing the benefits with the risks.

In at least one example, any one or more of the methods described herein further includes transmitting (e.g., by at least one processor) an alert to at least one computing device, the alert containing an answer for each of the one or more benefit-risk analysis (e.g., whether the benefit of a healthcare action, including, for instance, a medical product or service, outweighs the risk).

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 is a flowchart of a method of determining an appropriate risk metric for a healthcare action (e.g., providing a medical device and/or medical procedure), according to at least one example of the present disclosure.

FIG. 2 shows an equation communicating that benefit exceeds risk, according to at least one example of the present disclosure.

FIG. 3 is a flowchart of a method for using specific risk algebra rules to simplify various equations that define benefit and risk, according to at least one example of the present disclosure.

FIGS. 4A-4W show various equations that define benefit and risk, and their respective simplifications, to illustrate specific risk algebra rules, according to at least one example of the present disclosure.

FIG. 5 is a flowchart of a method for reviewing equations that define benefit and risk, according to at least one example of the present disclosure.

FIG. 6 is a flowchart of a method for determining a best healthcare action (e.g., a best medical treatment and/or therapy) from a list of potential alternative healthcare actions (e.g., alternative medical treatments and/or therapies), specifically that, for each R′+R, R′+R<R^{&} or the healthcare action cannot be ethically performed, according to at least one example of the present disclosure.

FIG. 7 is a flowchart of a method for calculating patient-specific weights for a benefit-risk analysis, according to at least one example of the present disclosure.

FIGS. 8A-8C relate to time-varying risk, and show a three-dimensional plot of probability and severity of various risks over a period of time (FIG. 8A), and flowcharts of a method for modeling a benefit that does not begin until after a period of time has passed (FIGS. 8B-8C), according to at least one example of the present disclosure.

FIG. 9 is a flowchart of a method for estimating the uncertainty associated with a benefit-risk analysis, according to at least one example of the present disclosure.

FIG. 10 is a block diagram of a computing system for identifying and/or calculating benefits of a healthcare action (e.g., a product and/or service, including, for instance, a medical device and/or a medical procedure), and for comparing the benefits with the risks, according to at least one example of the present disclosure.

FIG. 11 is a block diagram of a computing device, according to at least one example of the present disclosure.

FIG. 12 shows an example of a system for implementing certain aspects of the present technology.

FIG. 13 shows a further example of a system for implementing certain aspects of the present technology.

FIG. 14 is a flowchart of a method for determining whether a healthcare action's benefits exceed its risks, according to at least one example of the present disclosure.

FIG. 15 is a table of probability and severity categories for the health conditions before applying a specific healthcare action, Table_{RB}, set forth in Example 1, according to at least one example of the present disclosure.

FIG. 16 is another table of probability and severity categories for the health conditions after the healthcare action, Table_{RA}, in FIG. 15, according to at least one example of the present disclosure.

FIG. 17 is a table of probability and severity categories for additional health conditions caused by the healthcare action, Table_{R}, set forth in Example 1, according to at least one example of the present disclosure.

FIG. 18 is another table of probability and severity categories for the health conditions in FIGS. 15-17, after they have populated an equation, according to at least one example of the present disclosure.

FIGS. 19A-19H show various equations that define benefit and risk, and their respective simplifications, for the situation set forth in Example 1, according to at least one example of the present disclosure.

FIG. 20 is a table of probability and severity categories for the health conditions set forth in Example 2, according to at least one example of the present disclosure.

FIG. 21 is another table of probability and severity categories for the health conditions in FIG. 20, according to at least one example of the present disclosure.

FIG. 22 is a table of probability and severity categories for additional health conditions set forth in Example 2, according to at least one example of the present disclosure.

FIG. 23 shows an equation that defines benefit and risk for the situation set forth in Example 2, according to at least one example of the present disclosure.

FIGS. 24A-24F show various equations that define benefit and risk, and their respective simplifications, for the situation set forth in Example 2, according to at least one example of the present disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical or metric parameters set forth herein are approximations that can vary depending upon the desired properties sought to be obtained. At the very least, and not as an attempt to limit the application of the doctrine of equivalents to the scope of any claims, each numerical or metric parameter should be construed in light of the number of significant digits and ordinary rounding approaches. Further, the terms “numeric” (or “numerical”) and “metric” are used interchangeably herein.

The words “comprise,” “comprises,” and “comprising” are to be interpreted inclusively rather than exclusively. Likewise the terms “include,” “including,” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

In general, the word “instructions,” as used herein, refers to logic embodied in hardware or firmware, or to a collection of software units, possibly having entry and exit points, written in a programming language, such as, but not limited to, Python, R, Rust, Go, SWIFT, Objective-C, Java, JavaScript, Lua, C, C++, or C#. A software unit may be compiled and linked into an executable program, installed in a dynamic link library, or may be written in an interpreted programming language such as, but not limited to, Python, R, Ruby, JavaScript, or Perl. It will be appreciated that software units may be callable from other units or from themselves, and/or may be invoked in response to detected events or interrupts. Software units configured for execution on computing devices by their hardware processor(s) may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. Generally, the instructions described herein refer to logical modules that may be combined with other modules or divided into sub-modules despite their physical organization or storage. As used herein, the term “computer” is used in accordance with the full breadth of the term as understood by persons of ordinary skill in the art and includes, without limitation, mainframe computers, servers, desktop computers, laptop computers, embedded computers, tablets, smartphones, handheld computing devices, and the like.

In this disclosure, references are made to users performing certain steps or carrying out certain actions with their client computing devices/platforms. In general, such users and their computing devices are conceptually interchangeable. Therefore, it is to be understood that where an action is shown or described as being performed by a user, in various implementations and/or circumstances the action may be performed entirely by the user's computing device or by the user, using their computing device to a greater or lesser extent (e.g. a user may type out a response or input an action, or may choose from preselected responses or actions generated by the computing device). Similarly, where an action is shown or described as being carried out by a computing device, the action may be performed autonomously by that computing device or with more or less user input, in various circumstances and implementations.

In this disclosure, various implementations of a computer system architecture are possible, including, for instance, thin client (computing device for display and data entry) with fat server (cloud for app software, processing, and database), fat client (app software, processing, and display) with thin server (database), edge-fog-cloud computing, and other possible architectural implementations known in the art.

There exists a significant need for methods and processes for identifying, determining, calculating, and/or quantifying the benefits present in a given situation (e.g., the choice between competing program directions, the provision of a product or service, including the provision of a healthcare action, such as, for instance, a medical device and/or medical procedure, etc.), and for comparing such benefits to the situation's risks.

The term “healthcare action,” at least as used herein, refers to any action taken in a healthcare setting and/or in any healthcare application. Accordingly, a “healthcare action” includes, but is not limited to, the use, provision, and/or application of any product, device, and/or article of manufacture to one or more human individuals (e.g., one or more patients). Such products, devices, and/or articles of manufacture include, but are not limited to, those that require regulatory or governmental approval by one or more governmental entities, such as the United States Food and Drug Administration (FDA), before sale and/or use on one or more human individuals (e.g., one or more patients). Non-limiting examples of such products, devices, and/or articles of manufacture include, for instance, any instrument or machine that operates, or is intended to operate, to treat, cure, prevent, mitigate, and/or diagnose any disease, medical condition, and/or symptom thereof. The term “healthcare action” also includes, but is not limited to, the use, provision, and/or application of any service and/or procedure that operates, or is intended to operate, to treat, cure, prevent, mitigate, and/or diagnose any disease, medical condition, and/or symptom thereof.

Accordingly, embodiments of the present disclosure generally provide an objective way to define, determine, and/or calculate or quantify the benefits of one or more healthcare actions (e.g., provision of one or more products, such as a medical device), provision of one or more services, such as a medical procedure). As used herein, the term “objective” does not necessarily imply that the aforementioned embodiments eliminate all uncertainty in determining whether the benefit of a product and/or service outweighs the risk, but rather that such embodiments provide methods to quantify the benefits and/or risks in a defined manner.

In at least one example, novel methods are presented to determine whether benefit exceeds risk. Generally, such methods are different from currently-available methods by establishing more objective, common metrics for measuring both benefits and risks. Such methods further avoid the mistake of mapping risk to numbers for ease of manipulation. Instead, the methods described herein work with benefit and risk metrics directly, without conversion to numbers.

Accordingly, the methods may comprise defining one or more equations that weigh benefit and risk. The methods may further comprise one or more risk algebra rules for simplifying the aforementioned one or more equations.

The methods described herein can be applied to various healthcare actions, including, for instance, any medical device, any medical service (e.g., one or more treatments), any user population (e.g., any patient population), and any number or type of risks or benefits.

In at least one example, the method comprises quantifying and/or calculating benefit of a healthcare action (e.g., the provision of product and/or service) via a metric that is also used to calculate risk of the aforementioned action (e.g., product and/or service). Such quantification and/or calculation thereby simplifies the problem of determining whether benefits or risks of the given action (e.g., product and/or service) are greater.

The term “therapy,” at least as used herein, refers to any healthcare action (e.g., any medical device and/or medical procedure) that is used individually or in combination to treat, cure, prevent, mitigate, and/or diagnose any disease, medical condition, and/or symptom thereof.

Generally, the term “benefit-risk” analysis is used herein, as opposed to “risk-benefit” analysis. It should be appreciated that, in the last few years, various stakeholders (e.g., regulators) have used the term “benefit-risk” analysis due to the recognition that industry-submitted documents incorporating “risk-benefit” analysis has analyzed risk with only fleeting attention given to benefits. It is often the case that regulators want to see more information about a healthcare action's (e.g., a product's) benefits and a thorough analysis of whether the benefit or risk is greater. As a result, regulators have begun using the term “benefit-risk” instead of “risk-benefit.” See, e.g., EU Regulation 2017/745, Article 2, Section 24; and the FDA's “Enhancing Benefit-Risk Assessment in Regulatory Decision-Making.”

### Determining Whether Benefit Exceeds Risk

The phrase “first, do no harm” is often ascribed to the Hippocratic Oath and is generally recognized as the starting point of ethical medicine. Given the complexities of the human body, it is not always obvious what actions help, and what actions harm, a patient. Therefore, even a goal as modest as “first, do no harm” can be challenging to meet.

Generally, many healthcare actions, including, but not limited to, products or services such as medical devices and/or medical procedures, can expose users (e.g., patients in the context of healthcare actions such as medical devices and/or medical procedures) to some amount of risk. Accordingly, if one focused on “risk” alone, and without also thinking about “benefit,” then the fact that one should avoid risks (e.g., in the healthcare action context by following a “first, do no harm” principle) and the fact that the use of any product and/or service exposes a user to some risk would mean that one would do nothing. However, the entire point of providing healthcare actions, including products and/or services such as, for instance, medical devices and/or medical procedures, is that the use, provision, and/or application of such healthcare actions should be better than doing nothing. For instance, with respect to healthcare actions such as medical devices and/or medical procedures, the combination of the right patient with the right medicine should be better than doing nothing to assist the patient with his or her condition or medical issue. Therefore, in order to strike a balance that acknowledges the constant presence of both “risk” and “benefit” requires a framework that the benefits outweigh the risks. In the context of such healthcare actions (e.g., medical devices and/or medical procedures), this could entail a revision of the “first, do no harm” principle to “first, ensure the benefits outweigh the risks.”

Thus, the present disclosure, including, but not limited to, various embodiments presented herein, are not limited to specific applications and/or fields, including one or more healthcare actions (e.g., use, provision, and/or application of medical devices and/or medical procedures). For instance, various embodiments may be applicable to any product and/or service in which benefits and risks must be weighed against one another, including, for example, consumer appliances, electrical devices, and/or any product and/or service that can potentially result in some danger to the user.

Various institutions have developed risk management processes and standards, including, for example, the Project Management Institute, the National Institute of Standards and Technology (NIST), and the International Organization of Standardization (ISO). For instance, ISO has developed various standards (e.g., ISO 31000 and, in particular, ISO 31000:2018) relating to risk management. In the non-limiting context of healthcare actions (e.g., medical devices and/or medical procedures), ISO 14971:2019+A11:2021, Medical devices-Application of risk management to medical devices states: “All stakeholders need to understand that the use of a medical device involves an inherent degree of risk, even after the risks have been reduced to an acceptable level.”

From a regulatory perspective of products and/or services, including those used in healthcare actions, the test of whether benefit exceeds risk determines whether: (1) a product can be sold for use on patients (e.g., per the Food and Drug Administration (FDA)'s “Factors to Consider When Making Benefit/Risk Determinations in Medical Device Premarket Approval and De Novo Classifications,” issued Aug. 30, 2019, and the European Union (EU)'s Regulation (EU) 2017/745 of the European Parliament and of the Council, Annex I), (2) quality system remediations are acceptable (e.g., per “Factors to Consider Regarding Benefit-Risk in Medical Device Product Availability, Compliance, and Enforcement Decisions,” issued on Dec. 27, 2016, available at the fda.gov website), and (3) post-market regulatory actions (e.g., ranging from an audit recommendation to shutting down a company) are taken.

Additionally, legal challenges (e.g., product liability lawsuits) can be brought against a company based on challenges to whether a product and/or service's benefit exceeds its risk.

In at least one embodiment of the present disclosure, the likely amount of user (e.g., patient) benefit of a healthcare action (e.g., a product and/or service such as, for instance, a medical device and/or medical procedure) as B and the likely amount of user risk of that healthcare action as R, then the statement that benefit exceeds risk can be written as:

$$B > R \qquad ( Equation 1 )$$

Ironically, despite the long-standing requirement from both the FDA and the EU requiring medical manufacturers show Equation 1 is true, there is no objective or structured approach to show that a healthcare action's (e.g., a medical device's and/or medical procedure's) benefit exceeds its risk.

With respect to the “risk” side of the equation, various tools can be used to help understand and measure “risk,” with millions of hours training individuals on how to use these tools effectively. A small, non-limiting, sampling of the tools that have helped one understand risk include, (1) Failure Mode Analysis (FMA), originating during World War II (which was later expanded to include the effects of a failure mode, as which time the acronym FMA was lengthened to FMEA (Failure Mode Effects Analysis)), (2) Fault Tree Analysis (FTA), originating during the 1960's, and (3) Risk Prediction for Surgery, available at the riskprediction.org.uk0 website, with elements from 2016.

With respect to the “benefit” side of the equation, there have not been similar advances. For instance, there are no methods for measuring benefit that are analogous to FMEA, FTA, Risk Prediction, etc. One of the closest things to a universal metric for benefit is “financial units” (e.g., dollars, Euros, Yen, etc.). However, this metric is not suited to general application (including, for instance, to healthcare actions such as, e.g., medical devices and/or medical procedures) because regulators properly emphasize the user's welfare, and not financial considerations, to be the focus of risk management. Further, in contrast to various regulations (e.g., 21 Code of Federal Regulations (C.F.R.) § 820.30 (b)-(f)) that require device manufacturers to establish and maintain procedures on risk analysis, there are no regulatory requirements to establish or maintain procedures on benefit analysis.

In most benefit-risk analyses, the benefit and risk are stated in independent and unrelated manners. For instance, in the FDA Guidance on Benefit-Risk Analysis, the statements regarding risks and benefits discuss completely different topics, with no obvious way to compare them. Example 1 of this guidance discusses an “aesthetic device.” The benefit for this device is simply stated as “moderate,” with “some patients . . . [seeing] long-term aesthetic improvement” and the risks were stated as “adverse events of varying severity.” These statements have no way of being accurately and/or quantitatively compared, making it difficult to defend statements that benefit exceeds risk or vice versa.

Additionally, with respect to metrics such as changes in a patient's lifespan, one cannot currently measure the benefit and risk of healthcare actions (e.g., medical devices and/or medical procedures) by measuring changes in the patient's lifespan for risks and benefits that do not impact the patient's lifespan in a measurable manner (e.g., changes in the patient's lifespan are unlikely to be useful for acute-care healthcare actions). Further, even for medical conditions that do impact a patient's lifespan, measuring this effect will, necessarily, take years to decades. This will dramatically increase the cost of risk management, which may be impractical.

The Quality-Adjusted Time without Symptoms and Toxicity is another method that uses the same metric to measure both benefit and risk, where the metric is the time lost due to a healthcare action subtracted from the time gained from the action. While this example uses the same metric to measure both benefit and risk, the same problems exist as those mentioned above with respect to “changes in the patient's lifespan” metric.

Other tools that suffer from similar drawbacks include, for instance, Incremental net health benefit (INHB), Multi-Criteria Decision Analysis (MCDA) (which uses a complex and statistically tricky model, with assigned weights that can bring subjectivity biases into the model), Number-Needed-to Treat (NNT), and Number-Needed-to-Harm (NNH).

When filing a Section 510 (k) submission to “demonstrate that the device to be marketed is as safe and effective, that is, substantially equivalent, to a legally marketed device,” manufacturers traditionally show whether their product's benefit exceeds its risk by using “equivalence tables” to show that their product's safety and efficacy (in other words, “risks” and “benefits”) is at least as good as a “predicate device” that the manufacturer has identified because the “predicate device's” safety and efficacy record is both well-established and accepted by regulatory bodies. By choosing a predicate device with an accepted benefit and risk and by showing that the manufacturer's device is at least as good as the predicate device, the manufacturer shows the benefit and risk of the device in the 510 (k) is also acceptable.

However, the first problem with this submission strategy is that it requires a similar product already exist. Secondly, regulators occasionally remove products that have been on the market for years. This practice will throw into question all 510 (k) approvals using this product in an equivalence table.

By contrast, at least one embodiment of the disclosure comprises a method to show benefit exceeds risk that can be used on both completely novel products (e.g., medical devices), and/or on elements of an existing product design that are novel, without the potential problem that changes in another product's regulatory status will impact the new product's status.

Ultimately, the proliferation of information discussing the use of benefit-risk methods and structured benefit-risk assessments has not resulted in a consistent way to utilize these findings, especially in medical device development. Existing approaches to show benefit exceeds risk have significant drawbacks, including excessive appeal to expert opinion (i.e., lack of objective standards), potentially waiting for decades to collect data, not applying to novel products or procedures, and being overturned by changes in the regulatory status of other products. Without a universal, structured approach that enables objective comparisons of benefit and risk for any healthcare action (e.g., any product and/or service such as, for instance, medical devices and/or medical procedures), it is impossible to consistently and confidently state whether the risks or benefits of that healthcare action are greater.

### Examining Benefit

The following is a non-limiting examination of “benefit” in evaluating healthcare actions (e.g., products and/or services such as, for instance, medical devices and/or medical procedures), including “patient benefit.”

Specifically, the FDA's definition of a patient, per the FDA's “Patient-Focused Drug Development Glossary,” is “[a]ny individual with or at risk of a specific health condition, whether or not he or she currently receives any therapy to prevent or treat that condition. Patients are the individuals who directly experience the benefits and harms associated with medical products.” This definition of a patient has two parts that align with therapeutic, diagnostic, and/or preventive medical care, specifically (1) “[a] ny individual with a specific health condition,” and (2) “[a] ny individual at risk of a specific health condition.”

The medical benefits and risks of a therapeutic healthcare action (e.g., a medical device and/or medical procedure used for therapeutic purposes) can be fairly self-evident, namely, the benefit is that the health condition is improved, either partially or fully. The risk of such a therapeutic healthcare action is that it might cause some new harm (e.g., an infection) or that the health condition's improvement is not as extensive as usual.

The medical benefits of a diagnostic healthcare action (e.g., a medical device and/or medical procedure used for diagnosis purposes) can also be fairly self-evident, namely the presence or absence of a health condition is accurately identified. However, the scope of risks for a diagnostic healthcare action can be more subtle. Not only is there a chance that a diagnostic healthcare action might cause some new harm (e.g., an infection), but there are additional risks of not reporting a health condition that is present and reporting a health condition that is not present. This last option can lead to still more risks, including, for instance (a) performing an unnecessary therapeutic procedure, or (b) avoiding performing unnecessary therapeutic procedures by performing additional diagnostic tests, and the like. Further, even if additional diagnostic procedures eventually sort out the proper diagnostic result, this can result in months of therapeutic delays as successive conflicting diagnostic results are resolved to reach a single, confirmed result.

The medical benefits and risks of a preventative healthcare action (e.g., medical device and/or medical procedure used for preventative purposes) mirrors the benefits and risks of a therapeutic healthcare action. The benefit of the preventative healthcare action is successfully avoiding the undesirable health condition, either partially or fully. The risk of the preventative healthcare action is that it might cause some new harm or that the health condition occurs despite the preventative healthcare action.

Thus, whether the purpose of a healthcare action is therapeutic, diagnostic, or preventative, every such healthcare action exposes the patient to risk. In other words, in at least one embodiment of the disclosure, for every healthcare action,R>0.

Since R>0 for every healthcare action, and, per Equation 1, B>R for an ethical healthcare action, then the amount of benefit a patient is expected to receive from a given healthcare action holds a very special role in medicine: In at least one embodiment of the disclosure, the likely “benefit” of a healthcare action establishes an upper limit on the likely amount of “risk” that a patient can ethically be exposed to from the healthcare action; in other words, B>R.

In at least one embodiment, a variable H can be defined as the likely amount of harm from a specific health condition, then H≥B because a given healthcare action (a) cannot improve (or “benefit”) the patient's health by more than the amount of harm caused by a specific health condition (i.e., H is the maximum possible value for B, or H=B), or (b) may improve the patient's health, B>0, without completely curing the patient, so H>B.

Accordingly, in at least one embodiment, the definition of H means that the likely “harm” of a medical condition establishes an upper limit on the likely amount of “benefit” that a patient can ethically be exposed to from the healthcare action; that is, H≥ B.

If one considers an individual that does not meet the FDA's definition of a patient, than that person simultaneously does not currently have a health condition and is not at risk of a health condition in the future (i.e., for this person, H=0). This hypothetical person can be referred to as “100% healthy,” and everyone that does meet the FDA's definition of a patient can be referred to as “<100% healthy.”

In at least one embodiment, since H≥B and B>R, then, by the transitive properties of inequalities, H>R. Therefore, when H=0, it must also be true that both B=0 and R=0 for an ethical healthcare action. However, as discussed above herein, every healthcare action exposes the patient to some risk, i.e., R>0. Since R>0 for every healthcare action, but R=0 for an ethical healthcare action, then, in at least one embodiment, we can conclude that it is not ethical to use a healthcare action on anyone who is “100% healthy” (i.e., for whom B=0).

On the other hand, if someone either has, or is at least at risk of, a health condition, then their health is less than 100% (e.g., 90%). This loss of health presents an opportunity for the patient to benefit from a healthcare action. In at least one embodiment, this person can be ethically exposed to a healthcare action if the expected risk is less than the healthcare action's expected benefit.

### An Example of Benefit and Risk Tradeoffs

In many countries, including the U.S., there are many advertisements promoting botulinum toxin to treat migraines. Many such advertisements state that the toxin is intended to treat migraines in people with at least 15 headache days per month, where each headache lasts 4 hours a day or longer; that is, H=“at least 15 headache days per month, where each headache lasts at least 4 hours a day.” Since H≥B and B>R, then, by the transitive properties of inequalities, H>R. If it is assumed that, in order to maximize their patient population, makers of botulinum toxin set H as low as possible while still exceeding R, then R is only slightly smaller than H, so that R≈“15 headache days per month, where each headache lasts 4 hours a day.” This means, from the symptoms necessary for a patient to meet the intended use, that the healthcare action of migraines with botulinum toxin can be expected to cause significant, harmful side effects.

By extension, as the patient's health condition becomes more dire (i.e., as the amount of potential benefit increases), the amount of risk that can be tolerated in healthcare actions (e.g., a medical device and/or medical procedure) for treating the patient's health condition also increases. As a limit, patients with extreme health conditions (including, for instance, terminally ill patients) may legally and ethically be given experimental healthcare actions (e.g., experimental medical devices and/or experimental medical procedures) with unproven risks and benefits, typically through either controlled trials or compassionate use programs.

The following sections look at “benefit” to describe at least one embodiment of the disclosure that comprises an objective method of determining whether benefit exceeds risk in one or more healthcare actions, including, without limitation, products and/or services such as medical devices and/or medical procedures.

Describing “Benefit” with “Risk”

In at least one embodiment, since H is the likely amount of harm from a specific health condition, then, using the definition of B described above with respect to Equation 1, H−B is the likely amount of harm from the health condition that remains after the healthcare action (e.g., a medical device and/or medical procedure). That is, the benefit from a healthcare action is the same as the reduction in the patient's health condition from the healthcare action.

Additionally, juxtaposing a paraphrased definition of risk from ISO 14971 with the definition of H results in (1) “risk” is a combination of the probability of occurrence and severity of a specific harm, and (2) H is the likely amount of harm from a specific health condition.

Comparing the two statements above means that, in at least one embodiment, H is an estimate of the likely amount of harm from a specific health condition, and “risk” is a probabilistic (e.g., “likely”) estimate of the severity (e.g., “amount”) of harm from a specific property or indication (e.g., a “health condition”). This comparison results in the conclusion that, in at least one embodiment, H is the risk to someone's health from their health condition.

The significance of showing that H is a “risk” to someone's health is that it enables the use of tools that were initially developed for estimating the size and likelihood of a risk from a healthcare action in the novel application of estimating the size and likelihood of benefit from a health condition. Similar to H being the risk from a specific health condition, in at least one embodiment, H−B is the risk that remains from a health condition after the healthcare action is completed.

Although one cannot often directly observe the benefit of a healthcare action, one can, in at least one embodiment, observe the patient's health condition before and after the application of the healthcare action (e.g., before and after application of a medical device and/or medical procedure), and then compare the two observations to infer the benefit of the application of the healthcare action.

Accordingly, in at least one embodiment, the following variables are defined:
R_{B} as the likely amount of risk from the patient's health condition Before the healthcare action, and
R_{A} as the likely amount of risk from the patient's health condition After the healthcare action.

$$Thus , {R}_{B} = H , {R}_{A} = H - B , \\ and \\ B = {R}_{B} - {R}_{A} \qquad ( Equation 2 )$$

After defining three variables about risk, specifically, R_{B}, R_{A}, and R, at least one embodiment also uses the following IS/IS-NOT matrix, shown in Table 1 below, to clarify their definitions.

TABLE 1
IS/IS-NOT Matrix for Healthcare Actions

| | R_{B} | | R_{A} | | R | |
|---|---|---|---|---|---|---|
| | | IS | | IS | | IS |
| Characteristic | IS | NOT | IS | NOT | IS | NOT |
| Represents Patient Risk | X | | X | | X | |
| Represents Risk for the Patient Population | X | | X | | X | |
| Represents Risk for the Indications for Use | | X | | X | X | |
| Represents Risk for Contraindicated Uses | | X | | X | | X |
| Represents Unmitigated Risk | | X | | X | | X |
| Represents Mitigated Risk | X | | X | | X | |
| Represents Risk for Expected Misuse | | X | | X | X | |
| Represents Risk for Unexpected Misuse | | X | | X | | X |
| Represents Individual Residual Risks (e.g., per | | X | | X | X | |
| the definition of “Residual Risk” in ISO 14971) | | | | | | |
| Represents Risk from the Patient's Health | X | | | X | | X |
| Condition Before the Healthcare Action (e.g., | | | | | | |
| Medical Device and/or Medical Procedure) | | | | | | |
| Represents Risk from the Patient's Health | | X | X | | | X |
| Condition After the Healthcare Action | | | | | | |
| Represents Risk from the Healthcare Action | | X | | X | X | |
| Represents Risk from Side-Effects of the | | X | | X | X | |
| Healthcare Action | | | | | | |

### Establishing A Common Metric

An important feature of the definitions for Equation 1 and Equation 2 is that R_{B}, R_{A}, and R describe risks to the patient's health. Therefore, if an appropriate metric for risk to patient health can be established, then one can achieve the ultimate goal of measuring both benefit and risk with the same metric. This is important because it enables a direct comparison between the amount of risk and the amount of benefit in a significantly more objective manner than has traditionally been done for healthcare actions. In other words, at least one embodiment of the disclosure can show whether Equation 1, B>R, is true or not in an objective matter.

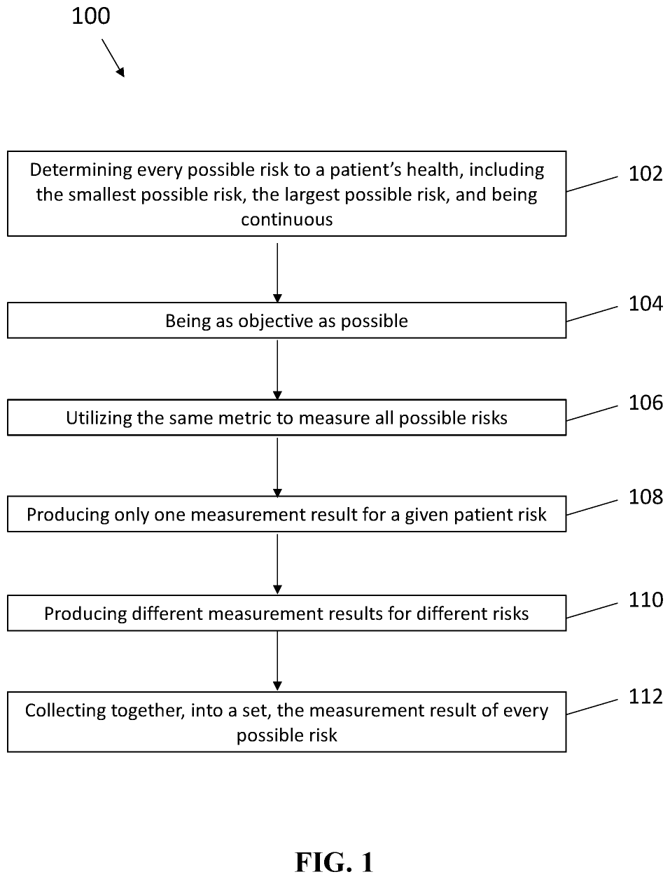

Turning now to FIG. 1, method 100 is shown that uses the following requirements and/or steps for an appropriate risk metric. The method comprises, at block 102, determining every possible risk to a patient's health, including (a) the smallest possible risk, (b) the largest possible risk, and (c) being continuous; that is, not having any gaps where risk is not measured. The method then comprises, at block 104, being as objective as possible. The method then comprises, at block 106, utilizing the same metric to measure all the possible risks. The method then comprises, at block 108, producing only one measurement result for a given patient risk. The method then comprises, at block 110, producing different measurement results for different risks. The method finally comprises, at block 112, collecting together, into a set, the measurement result of every possible risk.

In at least one embodiment, risk is a combination of severity and probability, specifically:

$${R}_{i} = ( {P}_{i} , {S}_{i} ) \qquad ( Equation 3 )$$

In Equation 3, Rᵢ is the i^{th} risk, Pᵢ is the probability of harm from the i^{th} risk, Sᵢ is the severity of harm from the i^{th} risk, and (Pᵢ, Sᵢ) is an ordered pair that holds Pᵢ and Sᵢ.

The following tables, specifically Table 2 and Table 3, are familiar to practitioners because they come from ISO 14971 and can be used to resolve the entire range of risk into five categories for severity and five categories for probability.

TABLE 2
Establishing Five Qualitative Severity Levels

| Common Terms | Possible Description |
|---|---|
| Catastrophic/Fatal | Results in death |
| Critical | Results in permanent impairment or irreversible |
| | injury |
| Serious/Major | Results in injury or impairment requiring |
| | medical or surgical intervention |
| Minor | Results in temporary injury or impairment not |
| | requiring medical or surgical intervention |
| Negligible | Results in inconvenience or temporary discomfort |

TABLE 3
Establishing Five Semi-Quantitative Probability Levels

| Common Terms | Examples of Probability Range |
|---|---|
| Frequent | ≥10⁻³ |
| Probable | <10⁻³ and ≥10⁻⁴ |
| Occasional | <10⁻⁴ and ≥10⁻⁵ |
| Remote | <10⁻⁵ and ≥10⁻⁶ |
| Improbable | <10⁻⁶ |

In at least one embodiment, the aforementioned producing only one measurement result for a given patient risk can be further refined with respect to severity and probability to ensure that significantly different risks do not have the same risk measurement.

Tables 2 and 3 above can be used to measure the risks from a given healthcare action (e.g., a given medical device and/or medical procedure), i.e., the risks in R. Accordingly, Table 3 does not resolve risks that occur more frequently than about 0.1% of the time. However, since the risks in R_{B} represent the risk to a patient's health before the healthcare action, at least some of the risks in R_{B} will always occur. Therefore, some of the risks in R_{B} will occur significantly more often than about 0.1% of the time. Using Table 3 as a risk metric, one would measure significantly different risks as the same if the risks occur more often than about 0.1% of the time. This would fail the abovementioned requirement and/or step to produce different measurement results for significantly different risks.

In order to correct the risk metric to treat risks with high rates of occurrence the same as risks for lower rates of occurrence, it is important to note that Table 3 uses decade-wide ranges for the center three risk categories. Therefore, in at least one embodiment of the disclosure, the high-occurrence end is extended while maintaining consistency with decade-wide probability ranges in the center of Table 3. The result is shown below in Table 4.

TABLE 4
Expanded Quantitative Table of Probability

| Common | Examples of | | |
|---|---|---|---|
| Terms | Probability Range | Equivalent Statements of Probability | |
| Expected | ≥10⁻⁰ and ≥10⁻¹ | ≥1.0 and ≥0.1 | ≥100% and ≥10% |
| Often | <10⁻¹ and ≥10⁻² | <0.1 and ≥0.01 | <10% and ≥1% |
| Frequent | <10⁻² and ≥10⁻³ | <0.01 and ≥0.001 | <1% and ≥0.1% |
| Probable | <10⁻³ and ≥10⁻⁴ | <0.001 and ≥0.0001 | <0.1% and ≥0.01% |
| Occasional | <10⁻⁴ and ≥10⁻⁵ | <0.0001 and ≥0.00001 | <0.01% and ≥0.001% |
| Remote | <10⁻⁵ and ≥10⁻⁶ | <0.00001 and ≥0.000001 | <0.001% and ≥0.0001% |
| Improbable | <10⁻⁶ | <0.000001 | <0.00001% |

Table 2 can also be improved, as described below herein with respect to at least one embodiment of the disclosure. Two of the five categories in that table briefly discuss “time.” At least one embodiment of the disclosure, set forth in further detail below, discusses an alternative way of accounting for the effect of “time” on risk. Further, when one reads the categories in Table 2, one thinks of physical harm to the patient. However, some healthcare actions are designed to mitigate mental and/or emotional harm to the patient (e.g., drugs to improve a patient's mental state or surgeries for aesthetic reasons only to improve a patient's sense of wellbeing). Thus, at least one embodiment of the disclosure, also set forth in further detail below, comprises a method to account for these factors.

Nevertheless, Tables 2 and 4 can be used in at least one embodiment of the disclosure to measure the severity of risk and the probability of risk, respectively.

### Organizing Risks

If Equation 2 is used to substitute R_{B} and R_{A} into Equation 1, and if R_{A} is added to each side of the equation, then the result is Equation 4 below:

$${R}_{B} > {R}_{A} + R \qquad ( Equation 4 )$$

The left-hand side of Equation 4 represents the patient's risk total before a healthcare action (e.g., a medical device and/or a medical procedure) is used to treat their health condition, and the right-hand side of Equation 4 represents the patient's risk total after a healthcare action is used to treat their health condition (i.e., the component of risk from the health situation that remains after the healthcare action and the component of risk from the healthcare action).

Therefore, Equation 4 states that the patient risk before a healthcare action is used to treat their health condition must be greater than the patient risk after that healthcare action is used to treat their health condition.

The three variables in Equation 4, namely R_{B}, R_{A}, and R, represent “sets of risks” and not “real numbers.” Thus, in at least one embodiment, Equation 4 can be further amended as follows. First, the addition sign (“+”) for real numbers is replaced by the union sign (“U”) for sets. Second, the greater than sign (“>”) for real numbers is replaced by a greater risk sign (“ [symbol] ”) for sets of risks. Just like “>” indicates that the number on the left-hand side of the inequality is larger than the number on the right-hand side of the inequality, so too does “ [symbol] ” indicate that the total amount of risk on the left side of the inequality is greater than the total amount of risk the right side of the inequality.

With the aforementioned substitutions, Equation 4 becomes:

$${R}_{B} {R}_{A} ⋃ R \qquad ( Equation 5 )$$

The three terms in Equation 5 each represent a set of risks, where, per Equation 3, each risk is represented by an ordered pair whose abscissa is the probability of harm and the ordinate is the severity of harm, and the probability of harm and severity of harm are text strings from Table 4 and 2, respectively.

At least one embodiment of the disclosure uses Equation 5 to organize sets of risks. The following table can be used to organize the individual risks to a patient within each set:

TABLE 5
Non-limiting example of a semi-quantitative 5 × 5 risk matrix

| | | Qualitative severity levels | | | | |
|---|---|---|---|---|---|---|
| | | | | Serious/ | | Catastrophic/ |
| | R | Negligible | Minor | Major | Critical | Fatal |
| Semi- | Frequent | | | | | |
| quantitative | Probable | R₁ | R₂ | | | |
| probability | Occasional | | R₄ | | R₅ | R₆ |
| levels | Remote | | | | | |
| | Improbable | | | R₃ | | |

Since this table is based on the five probability categories discussed in Table 3, and since seven probability categories are presented in Table 4, then Table 5, in at least one embodiment of the disclosure, can be extended by adding two more probability categories. The result is the following table which represents the entire range of risk with one of 35 combinations of probability and severity:

TABLE 6
Non-limiting example of a semi-quantitative 7 × 5 risk matrix

| | Qualitative severity levels | | | | |
|---|---|---|---|---|---|
| | | | Serious/ | | Catastrophic/ |
| R | Negligible | Minor | Major | Critical | Fatal |

| | |
|---|---|
| Semi- | Expected |
| quantitative | Often |
| probability | Frequent |
| levels | Probable |
| | Occasional |
| | Remote |
| | Improbable |

Per the definition of R and the right-most two columns of Table 1, R represents residual risks. For instance, if a healthcare action (e.g., a medical device and/or medical procedure) presents n risks to the patient (where n can represent any integer greater than zero), then the set of risks in R can be represented in any of three ways.

First, using n discrete risks, one can represent R as {R₁, R₂, R₃, . . . , Rₙ}.

Second, starting with n discrete risks, Equation 3 can be used to expand how the set of n discrete risks in R is represented, from {R₁, R₂, R₃, . . . , Rₙ} to {(P₁, S₁), (P₂, S₂), (P₃, S₃), . . . , (Pₙ, Sₙ)}. For instance, if a healthcare action exposes the patient to six risks and if:

$$R = \{ \begin{matrix}( Frequent , Minor ) , ( Occasional , Critical ) , ( Serious / Major , Improbable ) , \\ ( Occasional , Critical ) , ( Remote , Catastrophic ) , ( Probable , Negligible )\end{matrix} \}$$

then populating Table 6 with R results in the following:

TABLE 7
Populating Table 6 with R

| R | Negligible | Minor | Serious/Major | Critical | Catastrophic/Fatal |
|---|---|---|---|---|---|
| Expected | | | | | |
| Often | | | | | |
| Frequent | | (Frequent, Minor) | | | |
| Probable | (Probable, Negligible) | | | | |
| Occasional | | | | (Occassional, Critical) | |
| | | | | (Occassional, Critical) | |
| Remote | | | | | (Remote, Catastrophic) |
| Improbable | | | (Improbable, Serious/Major) | | |

Third, if the contents of each cell in Table 7 are replaced by a “count” of the number of risks that appear in that cell. Table 7 becomes Table 8, as shown below:

TABLE 8
Representing the Set of Risks in R

| | | | Serious/ | | Catastrophic/ |
|---|---|---|---|---|---|
| R | Negligible | Minor | Major | Critical | Fatal |
| Expected | 0 | 0 | 0 | 0 | 0 |
| Often | 0 | 0 | 0 | 0 | 0 |
| Frequent | 0 | 1 | 0 | 0 | 0 |
| Probable | 1 | 0 | 0 | 0 | 0 |
| Occasional | 0 | 0 | 0 | 2 | 0 |
| Remote | 0 | 0 | 0 | 0 | 1 |
| Improbable | 0 | 0 | 1 | 0 | 0 |

Similar tables can be used to represent the sets of risks in R_{B} and R_{A}. By substituting the tables that contain R_{B}, R_{A}, and R into Equation 5, a “table version” of Equation 5 is produced:

$${Table}_{{R}_{B}} \overbrace{>} {Table}_{{R}_{A}} ⋃ {Table}_{R}$$

The right-hand side of this equation can be simplified as follows:

$${Table}_{{R}_{A}} ⋃ {Table}_{R} = {Table}_{{R}_{A} ⋃ R} \qquad ( Equation 7 )$$

Standard matrix addition, i.e., by adding the risk count in corresponding cells of Table_{R}_{_{A}} and Table_{R} and putting the sum into the same cell in Table_{R}_{_{A}}_{∪R}, results in Equation 8:

$${Table}_{{R}_{B}} \overbrace{>} {Table}_{{R}_{A} ⋃ R} \qquad ( Equation 8 )$$

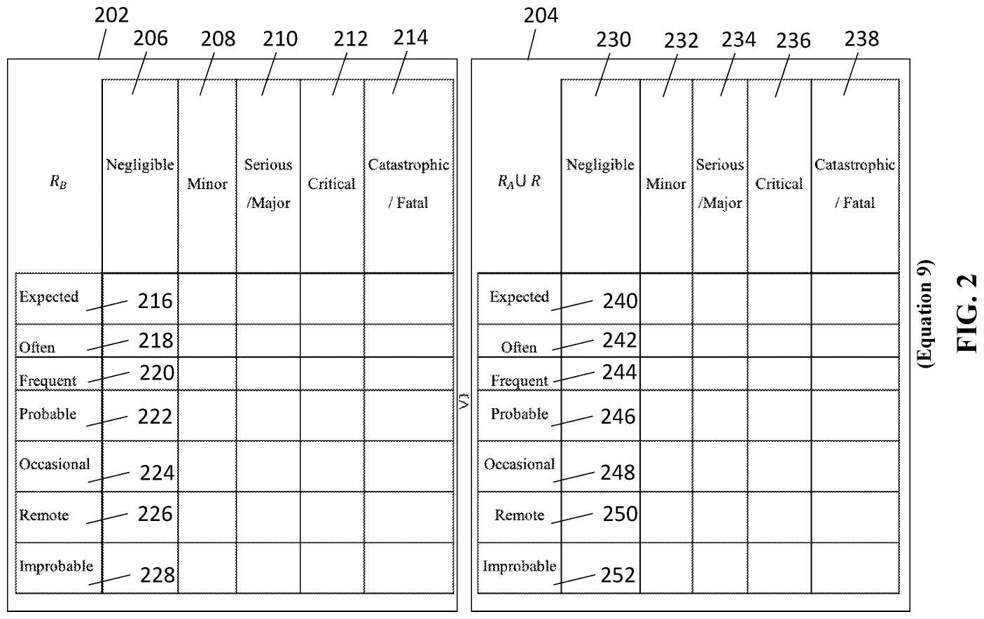

Equation 8 can be represented in “table form” as Equation 9, which is shown in FIG. 2. The left-hand side 202 of the equation represents R_{B}, while the right-hand side 204 represents R_{A}∪R. The left-hand side 202 has five severity levels/rankings as described above herein, specifically, “Negligible” in column 206, “Minor” in column 208, “Serious/Major” in column 210, “Critical” in column 212, and “Catastrophic/Fatal” in column 214. The left-hand side 202 further has seven probability levels/rankings as described above herein, specifically, “Expected” in row 216, “Often” in row 218, “Frequent” in row 220, “Probable” in row 222, “Occasional” in row 224, “Remote” in row 226, and “Improbable” in row 228. The right-hand side 204 has the same five severity levels/rankings as the left-hand side, namely “Negligible” in column 230, “Minor” in column 232, “Serious/Major” in column 234, “Critical” in column 236, and “Catastrophic/Fatal” in column 238. Finally, the right-hand side 204 has the same seven probability levels/rankings as the left-hand side, namely “Expected” in row 240, “Often” in row 242, “Frequent” in row 244, “Probable” in row 246, “Occasional” in row 248, “Remote” in row 250, and “Improbable” in row 252.

Thus, each side of Equation 9 (i.e., each table) represents a set of risks (the set R_{B} on the left-hand side of Equation 9 and the set R_{A}∪R on the right-hand side of Equation 9). Further, each cell in each table in contains a “count” of the number of risks with that cell's combination or probability and severity. Each count can be obtained by the process shown above to use Table 7 to obtain a count in Table 8 of the risks in Equation 6.

### Implementation

Equation 9 evolved from Equation 1, as described above herein. The following explains four rules of risk algebra that can be used to further simplify Equation 9. Since these rules are designed to not change whether the inequality in Equation 9 is true or not, if the simplified version of Equation 9 presented below herein is true, then Equation 1 is also true; that is, the benefit of a healthcare action (e.g., a device and/or service such as, for instance, a medical device and/or medical procedure) outweighs the risks.

In at least one embodiment of the disclosure, Equation 9 is simplified to determine whether the benefit exceeds the risk, or vice versa. As an example, if one wishes to solve the equation 3x²+7=55 for the number, x, the steps are:

| Equation | Simplification Step |
|---|---|
| 3x² + 7 = 55 | Original Equation − No |
| | simplification |
| 3x² + 7 − 7 = 55 − 7 | Subtract a number from both sides |
| 3x² + 0 = 48 | Simplify the Addition on both sides |
| $\frac{3 {x}^{2}}{3} = \frac{48}{3}$ | Divide by a number on both sides |
| $\frac{3}{3} {x}^{2} = 16$ | Simplify the Division on both sides |
| $\sqrt{{x}^{2}} = \sqrt{16} \\ x = 4$ | Take the square root of both sides The final answer − No simplification |

That is, to simplify an equation with numbers, as long as one does the same “thing” to both sides of the equation, that “thing” does not change the equation's equality. With respect to Equation 9, as long as the same risk is added, subtracted, or rearranged in the same way, to both sides, the equation remains true.

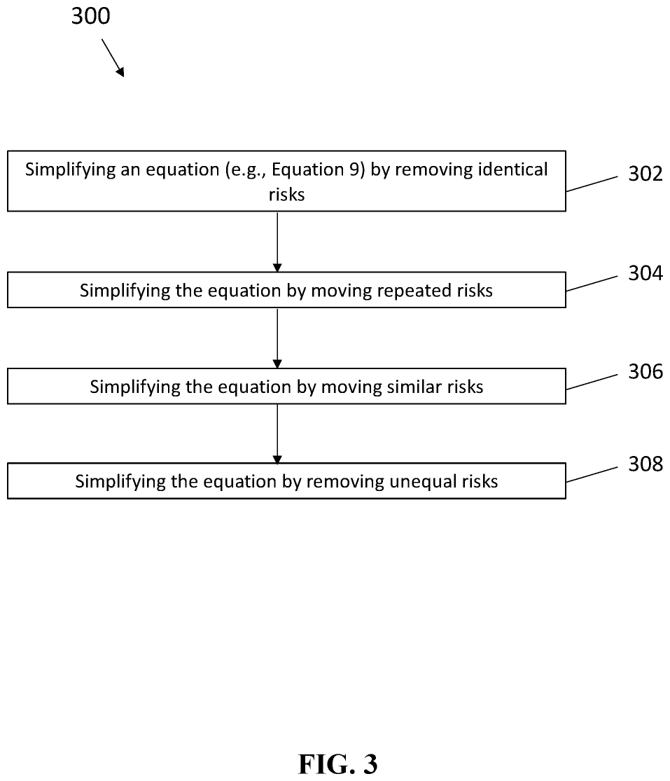

The following are four rules that can be used for simplifying Equation 9. The first two rules simplify the equation by (1) removing identical risks, and (2) removing unequal risks. The last two rules simplify the equation by (3) moving repeated risks, and (4) moving similar risks. Thus, FIG. 3 shows a method 300 according to at least one embodiment of the disclosure that encompasses the above for risk algebra rules. The method 300 may comprise, at block 302, simplifying an equation (e.g., Equation 9 as shown in FIG. 2) by removing identical risks. The method 300 may further comprise, at block 304, simplifying the equation by moving repeated risks. The method 300 may further comprise, at block 306, simplifying the equation by moving similar risks. The method 300 may further comprise, at block 308, simplifying the equation by removing unequal risks. Depending on the specific benefit-risk analysis and the specific equation to be simplified, one or more of these steps may be used more than once, or not at all.

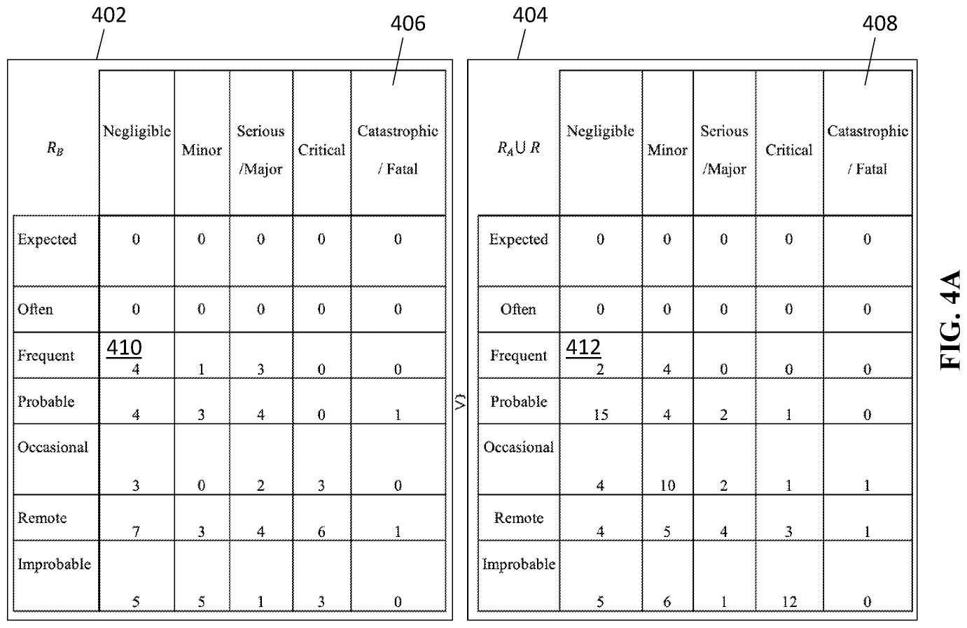

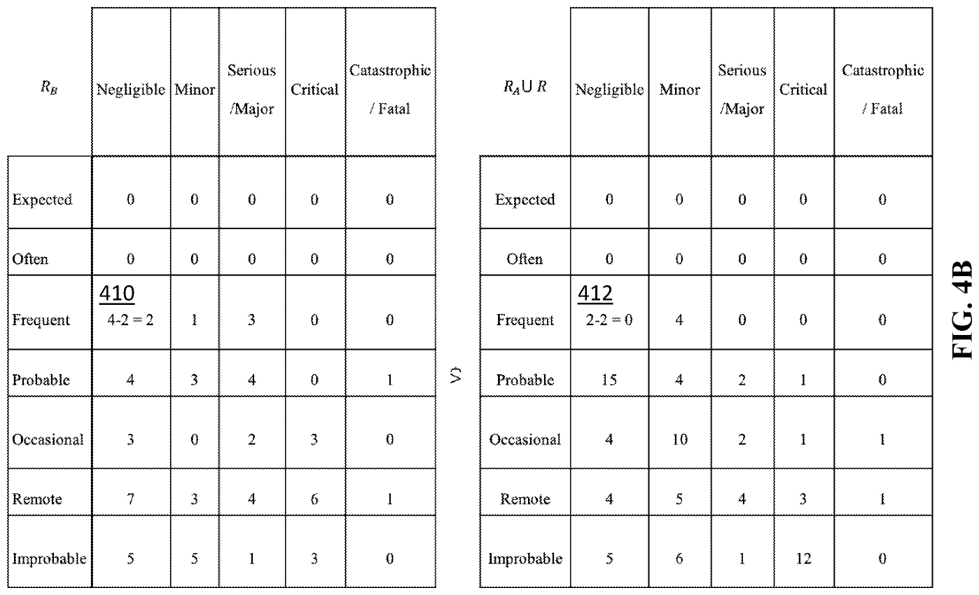

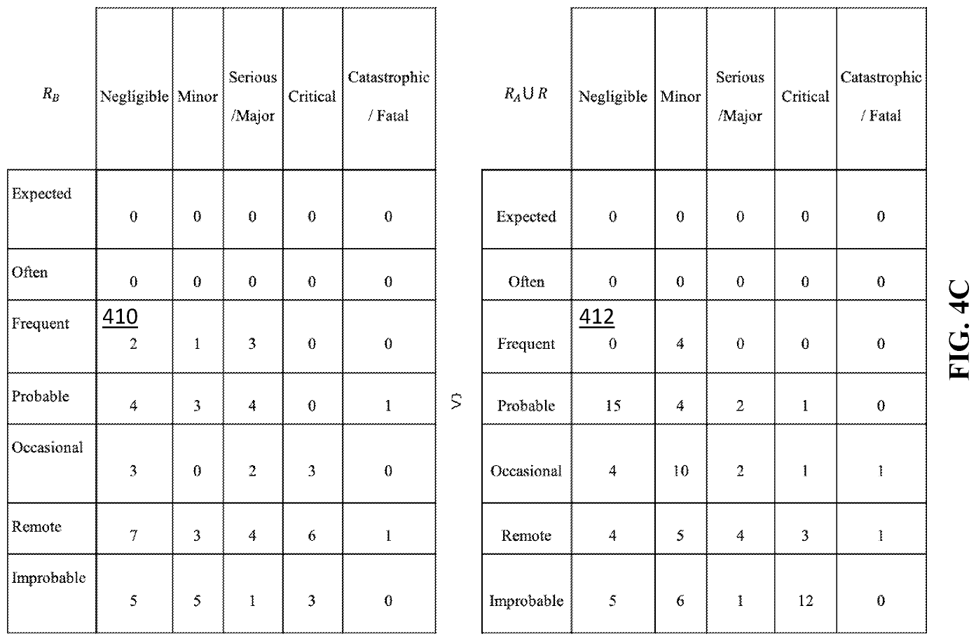

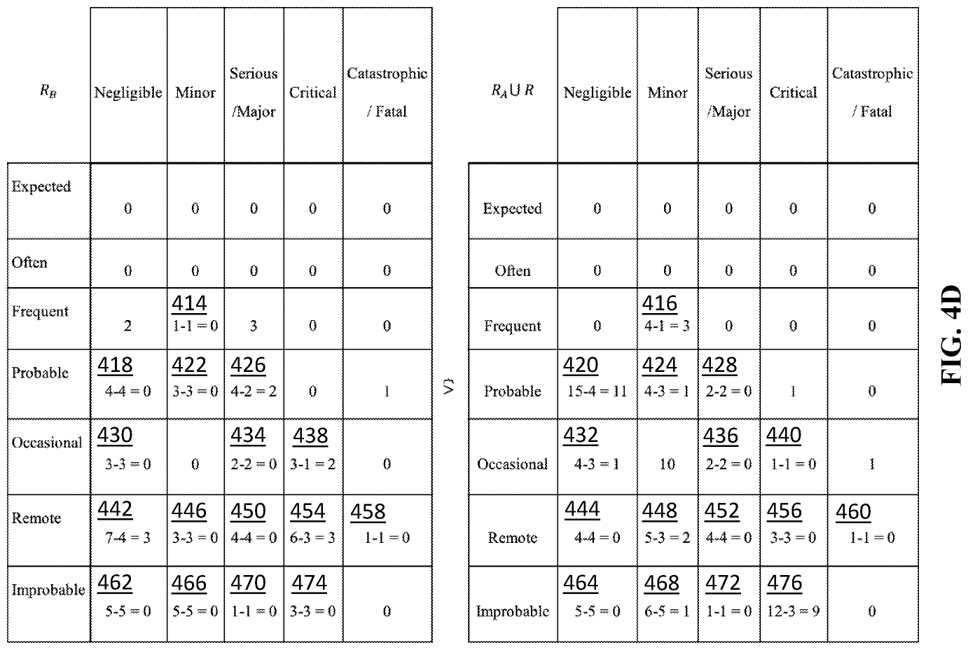

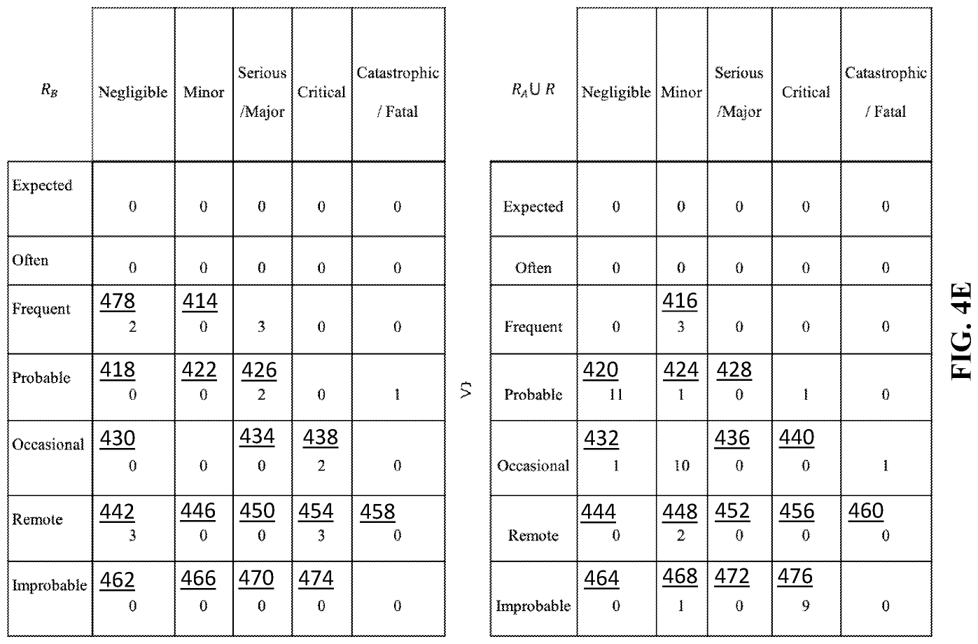

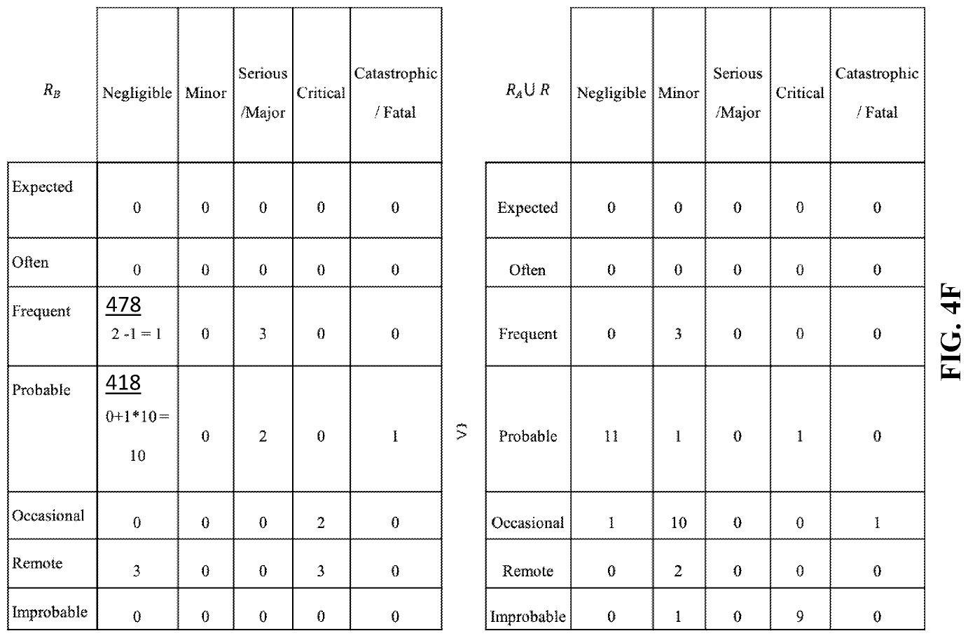

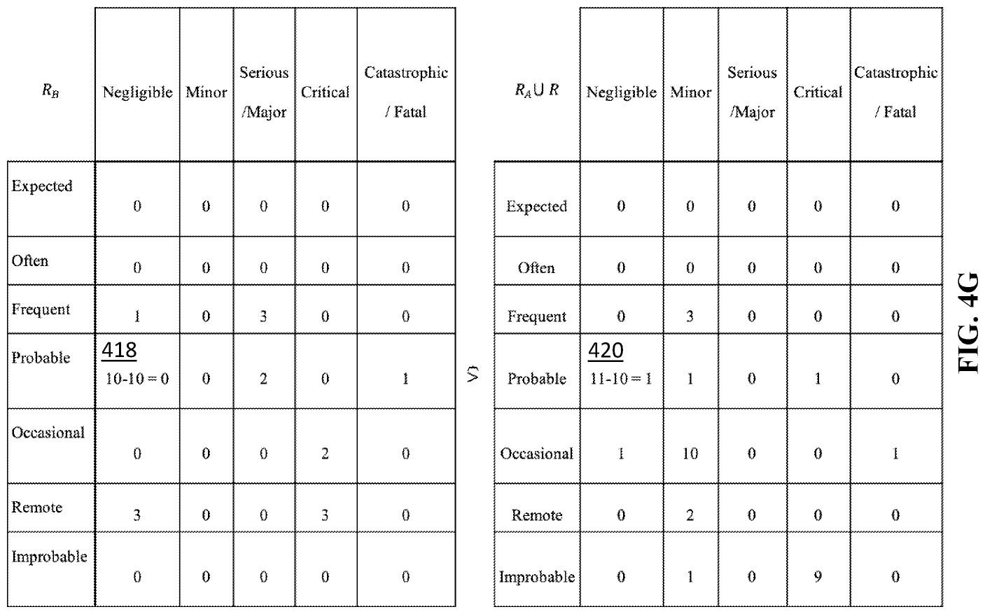

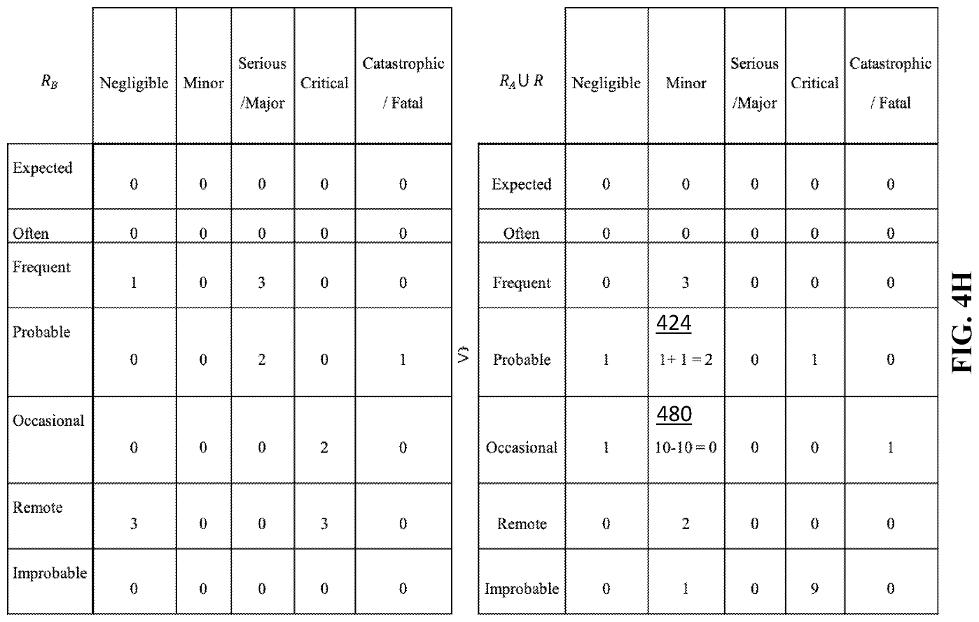

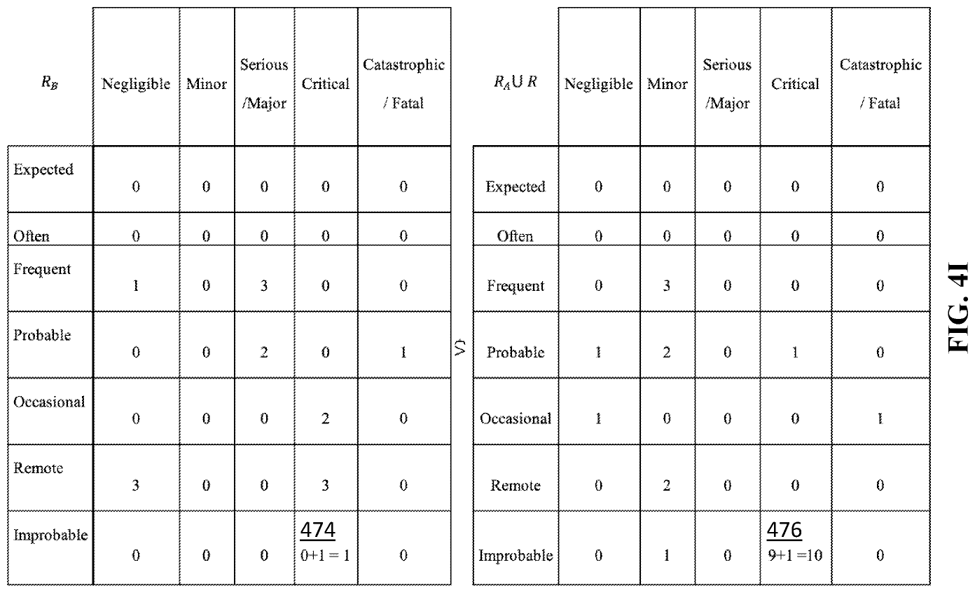

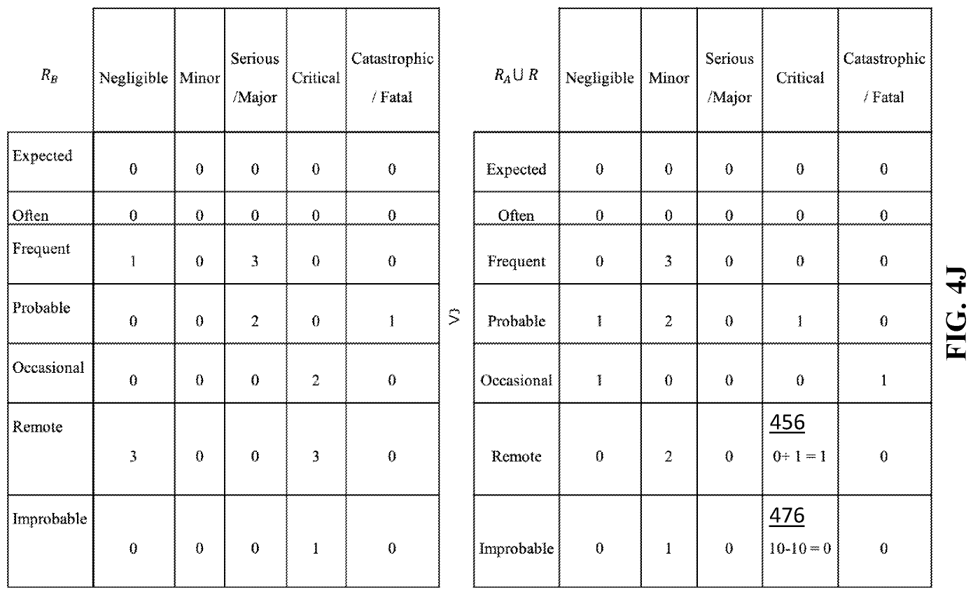

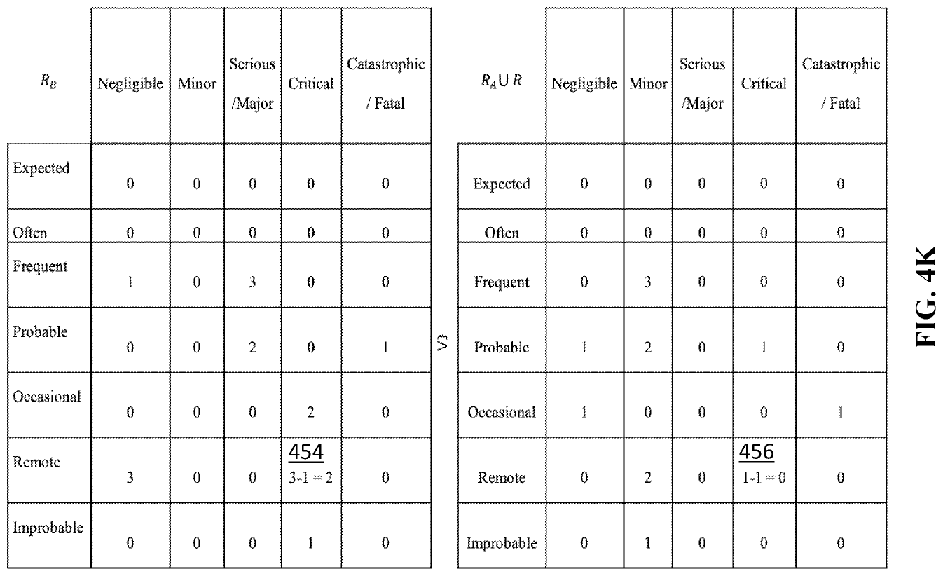

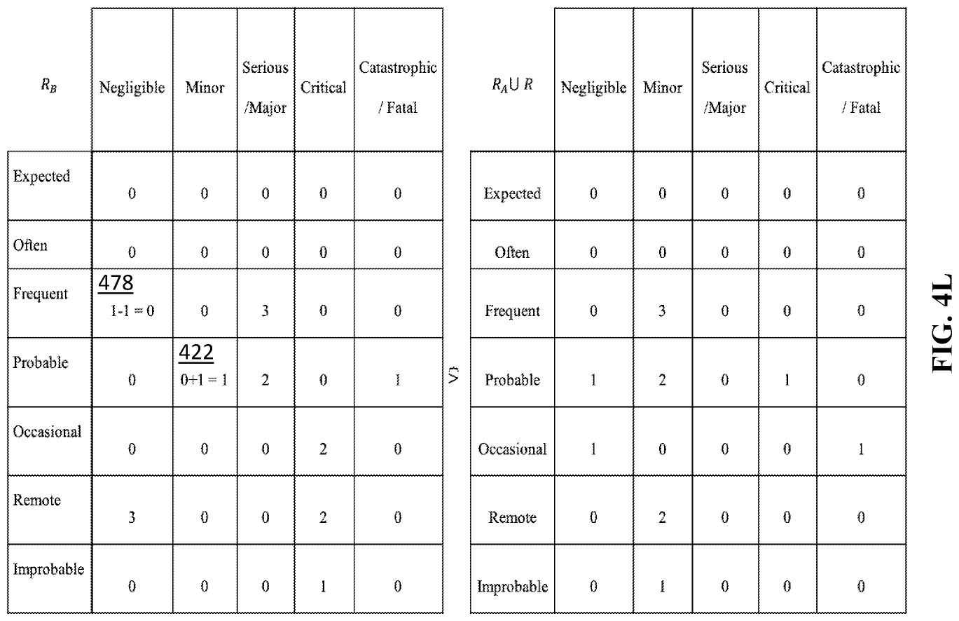

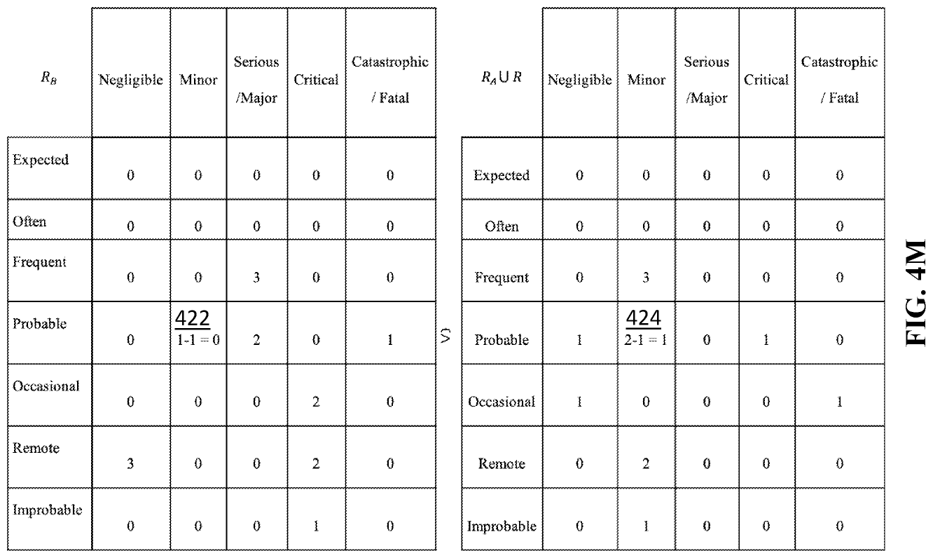

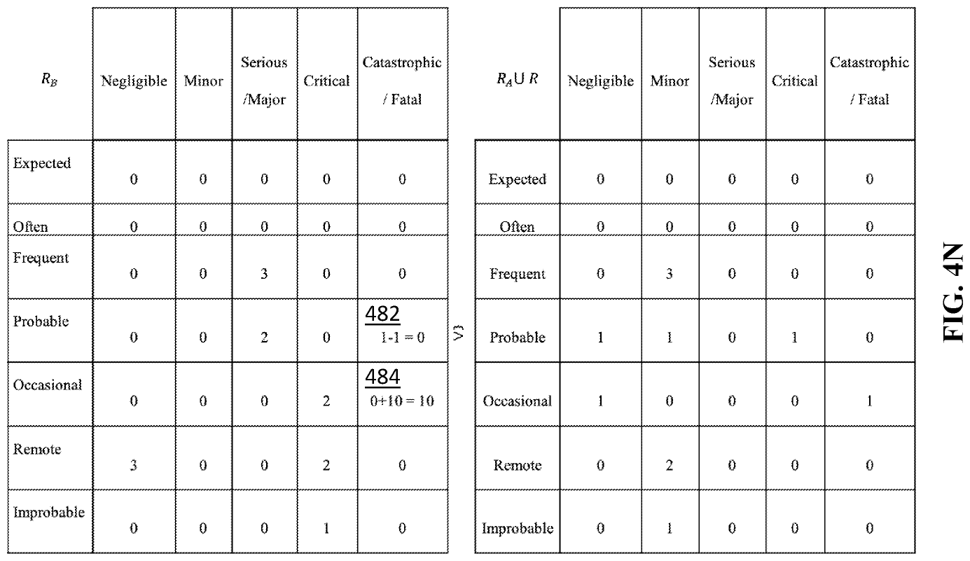

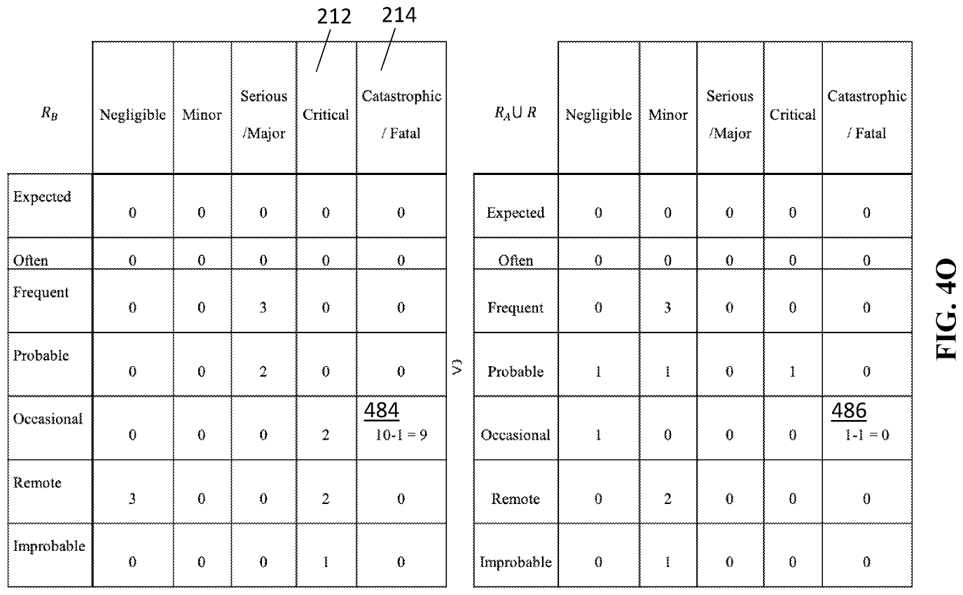

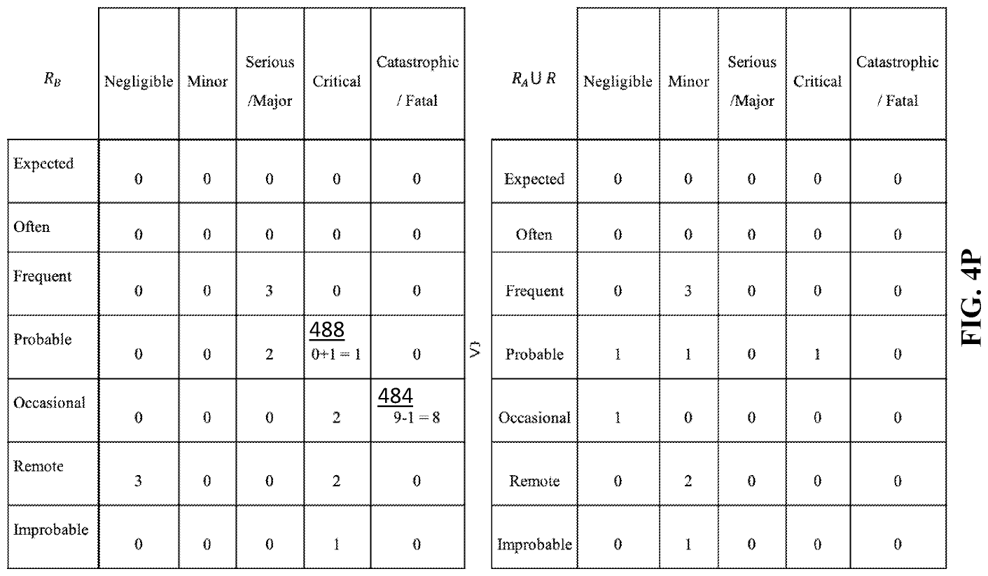

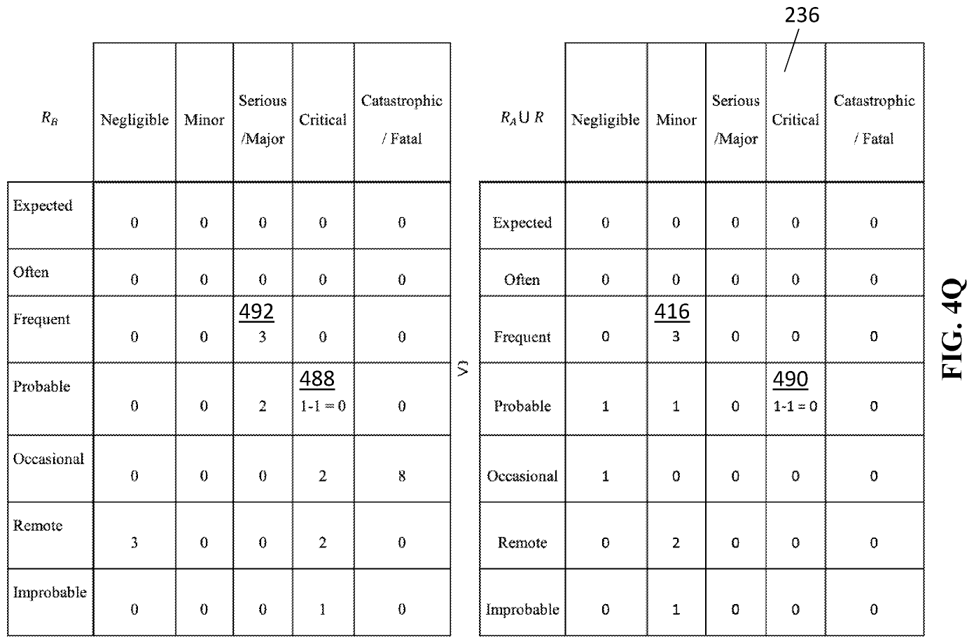

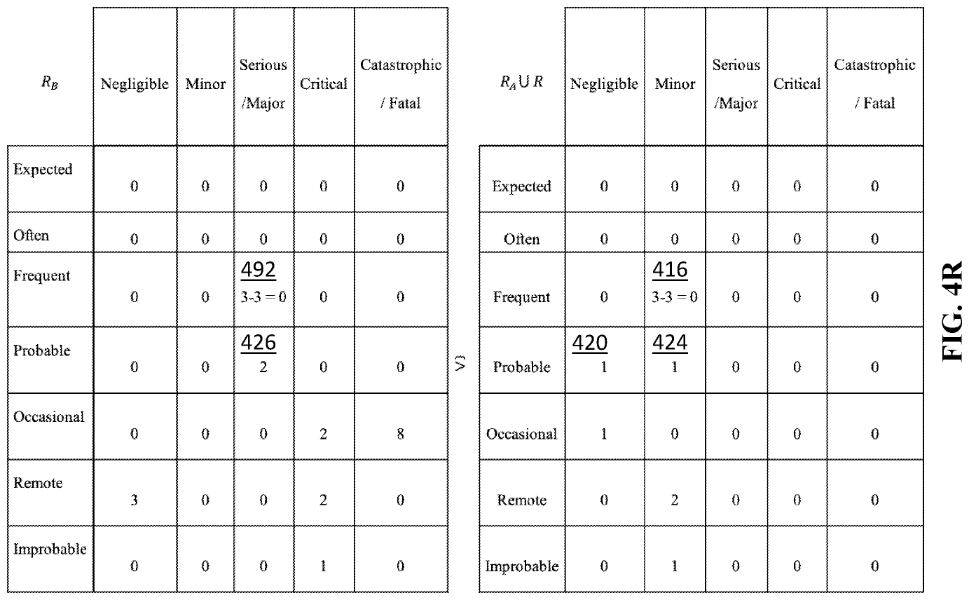

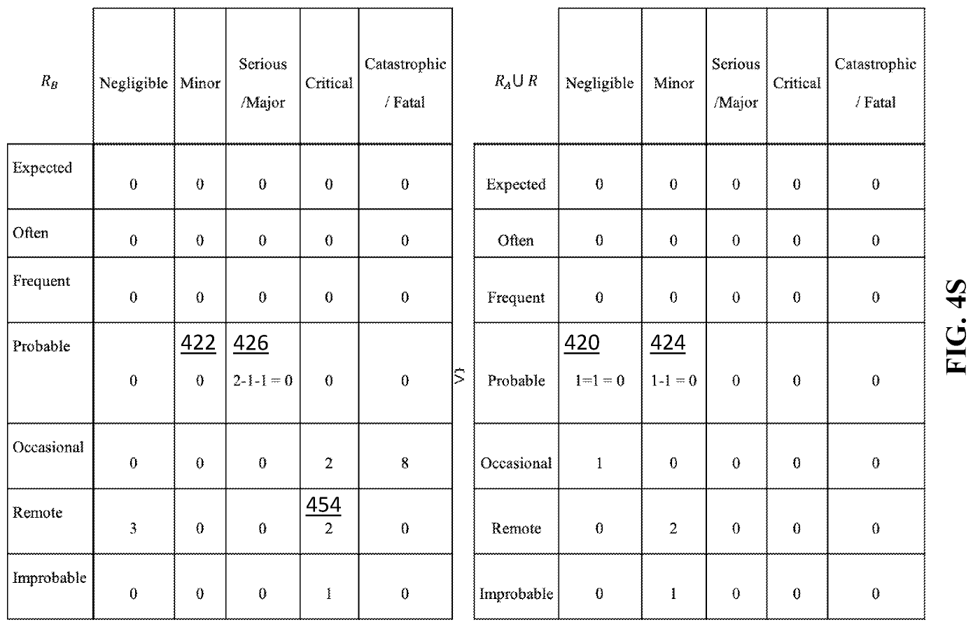

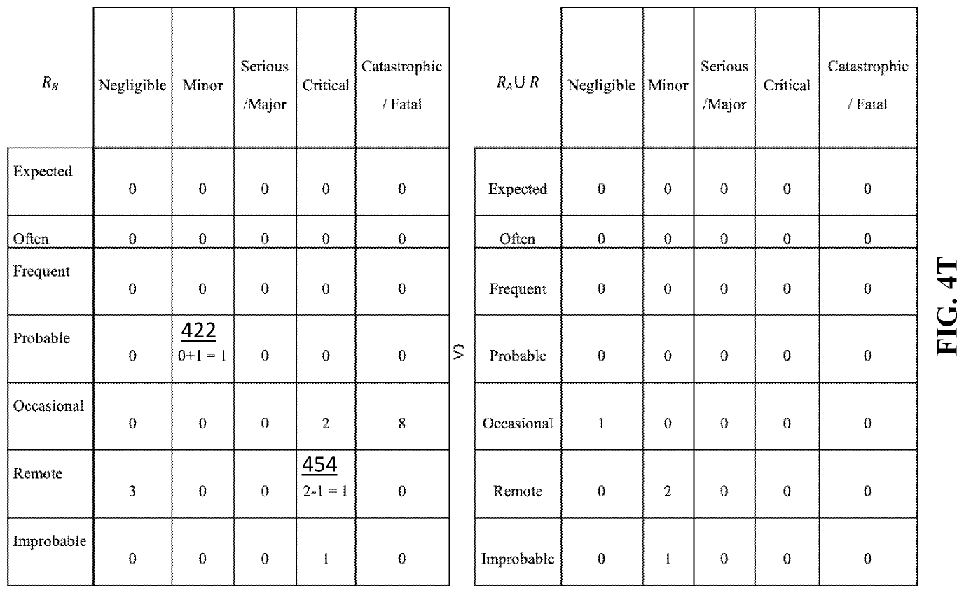

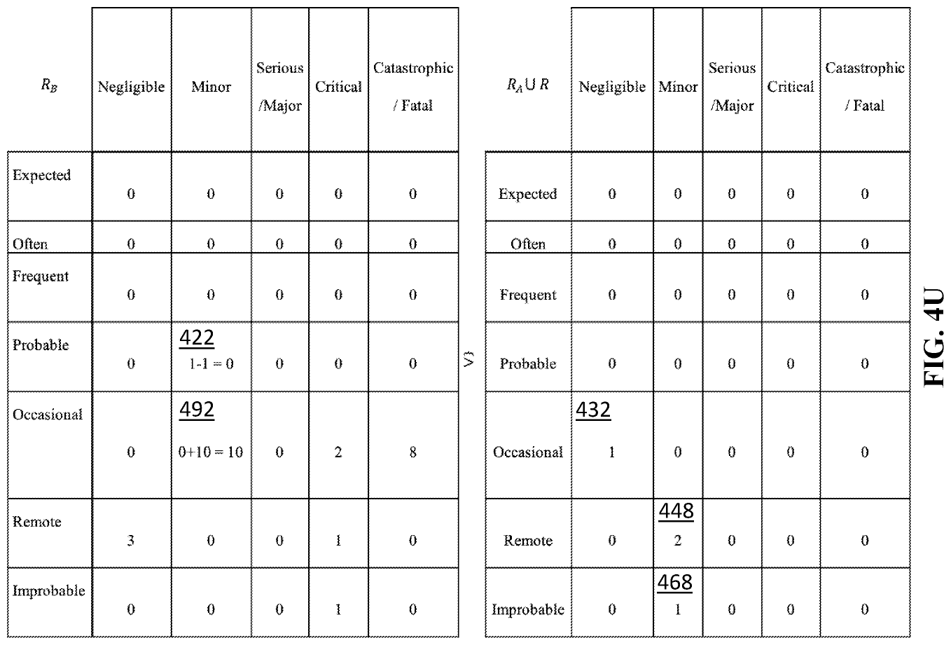

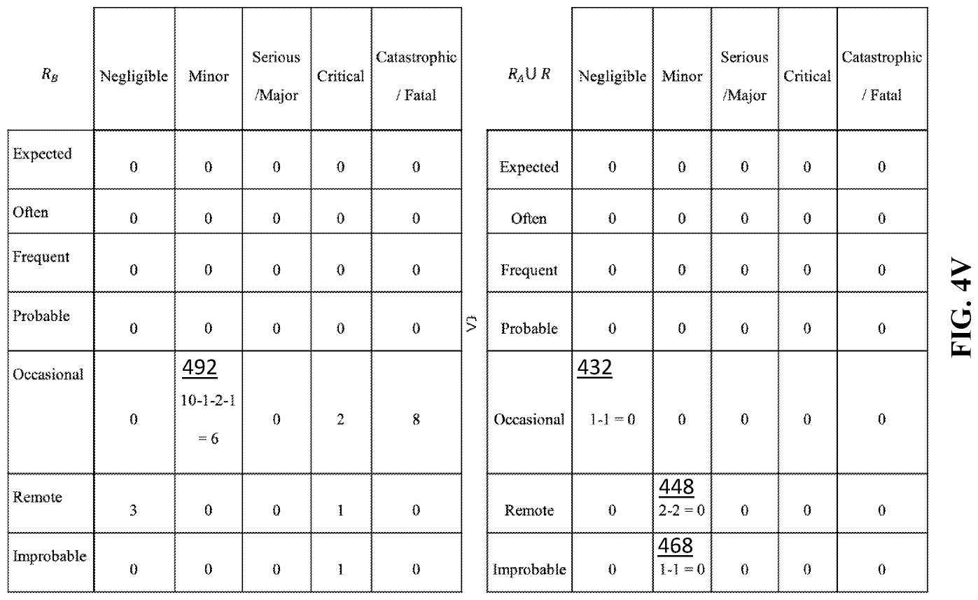

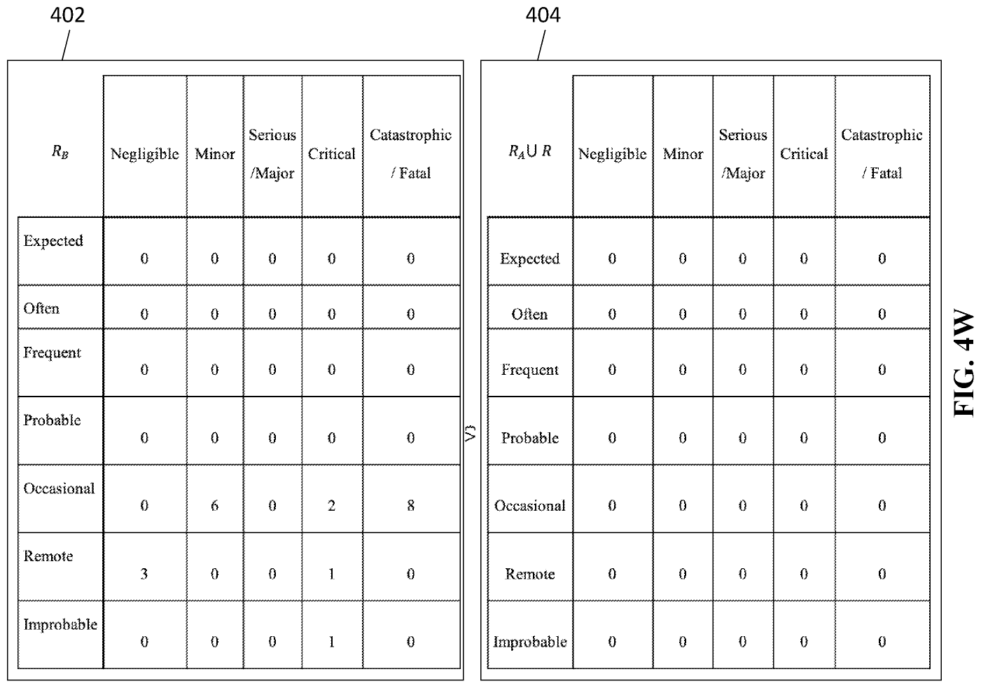

FIG. 4A shows Equation 10, which is a non-limiting example of specific “counts” in the cells of Equation 9. This example of counts is to illustrate how to use risk algebra and does not represent a true benefit-risk analysis. Examples of a true benefit-risk analysis are provided below herein in the “Examples” section.

As shown in Equation 10 in FIG. 4A, the sum of all the numbers on the right-hand side 402 of the equation is greater than the sum of the numbers on the left-hand side 404; that is, the right-hand side contains more risks than the left-hand side. Additionally, both sides of the equation contain the same number of risks in the “Catastrophic/Fatal” columns 406 and 408. That is, column 406, on the left-hand side of the equation, contains two total risks, while column 408, on the right-hand side of the equation, also contains two total risks. These observations might lead to the conclusion that the risk after application of the healthcare action (e.g., a medical device and/or medical procedure) is higher than the risk before; i.e., the healthcare action's risk exceeds its benefit.

However, for specific analysis, risk algebra can be used in at least one embodiment of the disclosure to simplify Equation 10 using the four algebraic rules described below herein (e.g., as represented in FIG. 3).

### Removing Identical Risks

If the same ordered pair (Pᵢ, Sᵢ) appears on both sides of the inequality in Equation 9, then it makes sense that removing that “same” ordered pair on both sides does not change which side of the equation has the greater risk. That is, if b represents a single ordered pair of probability and severity, then {a, b} [symbol] {c, b} implies the simpler equation {a} [symbol] {c}.

In order to recognize that the same ordered pair (Pᵢ, Sᵢ) appears on both sides of the inequality in Equation 9, it is important to note that the number that appears in each cell of the equation represents the number of risks in each set that share the probability and severity, (Pᵢ, Sᵢ), of that row and column (respectively) of the matrix. Therefore, if one looks for all of the instances in Equation 9 where the same cell has a non-zero number on both sides of the equation, then it is possible to identify all of the instances where the same ordered pair appears on both sides of the inequality in Equation 9. Removing one (or two or more) of these ordered pairs from a given cell means that “1” (or “2” or more) risks are subtracted from the same cell on both sides of Equation 9.

Turning now to Equation 10, shown in FIG. 4A, it can be seen that cell 410, at the junction of “Frequent” and “Negligible,” has a count of four risks on the left-hand side of Equation 10 and a count of two risks on the right-hand side (cell 412). By applying the rule of “Removing Identical Risks” to both sides of Equation 10, two Frequent/Negligible risks can be removed from each side of Equation 10 without changing whether the equation is true. This can be achieved by subtracting “2” from the Frequent/Negligible cell on each side of Equation 10, as shown in Equation 11 in FIG. 4B. As can be seen, cell a value of “2” has been subtracted from cell 410 and from cell 412.

After the above manipulation is performed, Equation 12, shown in FIG. 4C, is the result. As can be seen, FIG. 4C is identical to FIG. 4B, with the exception that the counts in cells 410 and 412 have been updated to their new values of “2” and “0,” respectively.

Further, if all identical risks are removed in all of the possible places in Equation 12, then Equation 13, shown in FIG. 4D, is the result. Specifically, a value of “1” has been removed from both cell 414 and corresponding cell 416. Similarly, a value of “4” has been removed from cells 418 and 420, respectively. A value of “3” has been removed from cells 422 and 424, respectively. A value of “2” has been removed from cells 426 and 428, respectively. A value of “3” has been removed from cells 430 and 432, respectively. A value of “2” has been removed from cells 434 and 436, respectively. A value of “1” has been removed from cells 438 and 440, respectively. A value of “4” has been removed from cells 442 and 444, respectively. A value of “3” has been removed from cells 446 and 448, respectively. A value of “4” has been removed from cells 450 and 452, respectively. A value of “3” has been removed from cells 454 and 456, respectively. A value of “1” has been removed from cells 458 and 460, respectively. A value of “5” has been removed from cells 462 and 464, respectively. A value of “5” has been removed from cells 466 and 468, respectively. A value of “1” has been removed from cells 470 and 472, respectively. Finally, a value of “3” has been removed from cells 474 and 476, respectively.

After the subtractions in Equation 13 have been performed, the equation is simplified to Equation 14, shown in FIG. 4E.

Since R_{B} and R_{A} contain risks to the patient's health before, and after, respectively, application of the healthcare action (e.g., a medical device and/or medical procedure), and since R_{B} and R_{A} are on opposite sides of the inequality in Equation 9, the rule of “Removing Identical Risks” will eliminate all of the patient health risks that are not affected by the healthcare action. Therefore, depending on the depth of detail in the patient health model, the first application of “Removing Identical Risks” may eliminate quite a few risks on both sides of the inequality in Equation 9, just like what occurred in this case.

### Moving Repeated Risks

Since the rows in Equation 9 represent probability, which row holds the “count” for a risk can be changed if the severity remains unchanged and the number of occurrences of the risk are changed to maintain the same probability. That is, if a cell generally contains a count of n risks, then one can move some, or all, of the risks down one row by the following.

First, in the original cell, n can be reduced by any positive integer less than or equal to n, say i. Thus, the “count” in the original cell changes from n to n−i. Second, i can be multiplied by the factor used to decrease probability range from the original row to the row below, F_{P}. Third, i×F_{P} can be added to the “count” in the cell below the original cell.

For example, if Table 4 can be used to define the probability in each row of Equation 9, then, in the left-hand side of Equation 14, shown in FIG. 4E, the row below “Frequent” (i.e., “Probable”) has a probability range that is 1/10^{th} the probability range of “Frequent”; i.e., F_{P}=10. The Frequent/Negligible cell in the left-hand side of Equation 14 (cell 478) has a value of “2”; i.e., n=2. Thus, the count in cell 478 can be reduced by “1”; i.e., i=1. Finally, the count in the Probable/Negligible cell in the left-hand side of Equation 14 (cell 418) can be increased by “10”; i.e., i×F_{P}. The result of the above-mentioned is to change Equation 14 into Equation 15, shown in FIG. 4F.

Then, Equation 15 can be further simplified by removing “10” from cell 418 and corresponding cell 420, as shown in Equation 16 in FIG. 4G.

There are many variants on the “Moving Repeated Risks” rule. For instance, while the previous example moved a risk “count” down one row to increase the count of a risk, a risk count can also be moved up one row to reduce the count of risk, as is shown in Equation 17 in FIG. 4H. Specifically, the risk count in cell 480 has been removed, and the risk count in cell 418 has been added accordingly.

Further, identical risks can be added to both sides of a risk equation without changing whether the equation is true in order to, for instance, set up other risk algebra rules. For example, one can “add” an “Improbable/Critical” risk to both sides of Equation 17, resulting in the equation shown in FIG. 4I. Specifically, a count of “1” has been added to both cell 474 and corresponding cell 476.

Using the “Moving Repeated Risks” rule on the equation shown in FIG. 4I yields the equation shown in FIG. 4J. Specifically, the risk count in cell 476 has been removed, and the risk count in cell 456 has been added accordingly.

This equation shown in FIG. 4J can then itself be modified using the “Removing Identical Risks” rule to produce Equation 18, shown in FIG. 4K. Specifically, a count of “1” has been removed from both cell 454 and corresponding cell 456.

A fundamental problem with many currently-available risk metrics is that they assign sequential integers to each severity category; i.e., “1” for “Negligible” and “5” for “Catastrophic/Fatal.” This leads to the conclusion that three “Minor” harms are more important than one “Catastrophic/Fatal” harm, which is both untrue and offensive. A strength of the methods presented herein is that the two previous rules of risk algebra always work within the same severity level, so that comparisons of the relative importance of, for instance, three “Serious” harms and one “Catastrophic/Fatal” harm are avoided. However, the last two risk algebra rules, presented below herein, can work across severity levels, but do so in ways that avoid the untrue and offensive comparisons that are inevitable when severity levels are assigned numbers and risks are added together.

### Moving Similar Risks

The previous algebraic rule was built on the concept that the same risk can be represented in different cells, e.g., of Table 6, by moving the “count” for a risk to different rows within the same severity. This algebraic rule generalizes the concept of representing the same risk in different cells by moving the “count” for a risk across a band of cells with similar risks. In the following table, combinations of Severity and Probability with the same number and shading color have approximately the same amount of risk.

For example, a “Remote-Serious/Major” risk is in a cell of Table 9 with the number “4.” This means that the “count” for this risk could be moved to another cell within band number “4,” such as, for instance, “Occasional-Minor.”

An important consideration to ensure the “Moving Similar Risks” rule never changes a false equation to a true one is to construct risk metrics so that changes in the level of probability and severity have roughly the same impact on patient risk. Specifically, a change of one level of probability or severity have roughly the same impact on patient risk.

A second consideration is based on two core characteristics that matter when moving between severity categories: First, changes in severity category cause non-linear changes in patient risk, and second, severity changes increase monotonically from “Negligible” to “Catastrophic/Fatal.”

As a result of these characteristics, when using the “Moving Similar Risks” rule, it is important to avoid understating the risk by moving “Minor” risks to “Catastrophic/Fatal” risks. Instead, one should overstate the risk by moving risks from higher severities to lower severities without changing the number of risks. For example, moving n “Catastrophic/Fatal” risks to n “Critical” risks is conservative.

Additionally, since risk metrics cannot be constructed so that changes in the level of probability and severity have exactly the same impact on patient risk for all combinations of probability and severity, as few risks should be moved as necessary to simplify the equation, but no more. Further, risks should be moved down as few severities as necessary to simplify the equation.

By applying the “Moving Similar Risks” rule to Equation 18, shown in FIG. 4K, we can move the Negligible/Probable cell (cell 478) on the left-hand side of the equation, along the “4” risk band, to the Minor/Occasional cell (cell 422), as shown in the equation in FIG. 4L.

This change enables the use of the “Removing Identical Risks” rule to produce Equation 19, shown in FIG. 4M. Specifically, a risk count of “1” is removed from both cell 422 and corresponding cell 424.

All three of the above-mentioned risk algebra rules can now be combined to simplify the “Catastrophic/Fatal” risks and then the “Critical” risks on both sides of Equation 19. First, the “Moving Repeated Risks” rule can be used on the left-hand side of Equation 19 to produce the equation shown in FIG. 4N. Specifically, the risk count of cell 482 is removed, and the risk count of cell 484 is added accordingly.

Second, the “Removing Identical Risks” rule can be used on the “Occasional-Catastrophic/Fatal” cells, as shown in the equation shown in FIG. 4O. Specifically, a risk count of “1” has been removed from both cell 484 and corresponding cell 486.

In the above, all “Catastrophic/Fatal” risks have been eliminated from the right-hand side of the equation. Similarly, all “Critical” Risks can be eliminated from the right-hand side as well. First, the “Moving Similar Risks” rule can be used to move the risks in the “Catastrophic/Fatal” column (column 214 of the left-hand side of the equation shown in FIG. 4O), along the “7” risk band, to the “Critical” column (column 212). This simplification produces the equation shown in FIG. 4P. Specifically, a risk count of “1” has been removed from cell 484, and the same risk count has been added to corresponding cell 488, along the same risk band.

Then, the “Removing Identical Risks” rule can be used to simplify both sides of the equation shown in FIG. 4P to produce Equation 20, shown in FIG. 4Q. Specifically, a risk count of “1” has been removed from both cell 488 and corresponding cell 490.

As shown in Equation 20, all of the risks in the “Critical” column (column 236) have been eliminated from the right-hand side.

The simplifications discussed above herein have eliminated all of the risks from the right-hand side of Equation 20 that are greater than “Minor.” Equation 20 can continue to be further simplified to illustrate the fourth risk algebra rule, “Removing Unequal Risks.”

### Removing Unequal Risks

As described with reference to the “Removing Identical Risks” rule, {a, b} [symbol] {c, b} implies the simpler equation {a} [symbol] {c}. Considering the starting equation, {a, b} [symbol] {c, d}, assume |{x}| is a metric that measures the amount of risk in the set {x}. Since {b} is a set of risks that was removed from {a}, then {a}∩{b}=Ø. However, because {a}∩{b}=Ø, then |{a, b}|=|{a}|+|{b}|. Similarly, |{c, d}|=|{c}|+|{d}|. Therefore, {a, b} [symbol] {c, d} is equivalent to |{a}|+|{b}| [symbol] |{c}|+|{d}|. Since |{a}| is a lot of symbols, the symbology can be simplified so that a, b, c, and d represent the amount of risk in each set of risks. Thus, {a, b} [symbol] {c, d} can be simplified to a+b [symbol] c+d. With this symbology, we want to simplify a+b [symbol] c+d to a [symbol] c.

Since unequal risks are being removed, then either b [symbol] d or d [symbol] b, and each case will be considered below in turn.

First, for the case where b [symbol] d, if one begins with Equation 9 written as a [symbol] c, and remove b₁ from a (so, a₁=a [symbol] b₁) and remove d₁ from c (so, c₁=c [symbol] d₁), then a₁+b₁ [symbol] c₁+d₁ and b₁ [symbol] d₁ implies that a₁+(b₁ [symbol] d₁) [symbol] c₁, where b₁ [symbol] d₁ [symbol] 0. Therefore, when a₁+b₁>c₁+d₁ is simplified to a₁>c₁, the amount of risk on the left-hand side of the equation is reduced by the amount of b₁ [symbol] d₁.

If the previous manipulations are previous paragraph n times, then a [symbol] c becomes:

$${a}_{n} + {\sum }_{i = 1}^{n} ( {b}_{i} \overbrace{-} {d}_{i} ) \overbrace{>} {c}_{n}$$

and, simplifying the previous equation to aₙ [symbol] cₙ, the amount of risk on the left-hand side of the equation is reduced by Σᵢ₌₁ⁿ(bᵢ [symbol] dᵢ).

As n increases, one of two things will happen first. Either (1) cₙ=0; i.e., there are more risks to remove on the left-hand side of the equation, but no more risks to remove from the right-hand side of the equation, or (2) aₙ=0; i.e., there are more risks to remove on the right-hand side of the equation, but no more risks to remove from the left-hand side of the equation. If the first case is true, then Equation 9 is true. If, on the other hand, the second case occurs, then Equation 9 isn't necessarily false. It is also possible that Σᵢ₌₁ⁿ(bᵢ [symbol] dᵢ) grew larger than aᵢ [symbol] cᵢ. One way to tell which of these options occurred is to reverse the inequality in Equation 9 and simplify it again. Thus, in this new equation, d [symbol] b, so:

$${a}_{n} \overbrace{>} {c}_{n} + {\sum }_{i = 1}^{n} ( {d}_{i} \overbrace{-} {b}_{i} )$$

$${a}_{n} \overbrace{>} {c}_{n} + {\sum }_{i = 1}^{n} ( {d}_{i} {\overbrace{-}}_{i} )$$

and the result of the simplification will be different than it was the first time.

Second, for the case where d [symbol] b, if one begins with Equation 9 written as a [symbol] c, and remove b₁ from a (so, a₁=a [symbol] b₁) and remove d₁ from c (so, c₁=c [symbol] d₁), then a₁+b₁ [symbol] c₁+d₁ and b₁ [symbol] d₁ implies that a₁ [symbol] c₁+(d₁ [symbol] b₁), where d₁ [symbol] b₁ [symbol] 0. Therefore, when a₁+b₁>c₁+d₁ is simplified to a₁>c₁, the amount of risk on the right-hand side of the equation is reduced by the amount of d₁ [symbol] b₁.

If the previous manipulations are repeated n times, then a [symbol] c becomes:

$${a}_{n} \overbrace{>} {c}_{n} + {\sum }_{i = 1}^{n} ( {d}_{i} \overbrace{>} {b}_{i} )$$

and, simplifying the previous equation to aₙ [symbol] cₙ, then the amount of risk on the right-hand side of the equation is reduced by Σᵢ₌₁ⁿ(dᵢ [symbol] bᵢ).

As n increases, one of two things will happen first. Either (1) cₙ=0; i.e., there are more risks to remove on the left-hand side of the equation, but no more risks to remove from the right-hand side of the equation, or (2) aₙ=0; i.e., there are more risks to remove on the right-hand side of the equation, but no more risks to remove from the left-hand side of the equation. If the first case occurs, Equation 9 isn't necessarily false. It is also possible that Σᵢ₌₁ⁿ(bᵢ [symbol] dᵢ) grew larger than cᵢ. If the second case occurs, Equation 9 also isn't necessarily false. It is also possible that Σᵢ₌₁ⁿ(dᵢ [symbol] bᵢ) grew larger than aᵢ [symbol] cᵢ.

Comparing the two previous situations, either b [symbol] d or d [symbol] b, the only situation that enables us to conclude whether Equation 9 is true or not is the case where b [symbol] d. Therefore, in at least one embodiment of the disclosure, Equation 9 will be simplified by removing unequal risks only if b [symbol] d is true.

When using the “Removing Unequal Risks” rule, if the simplified version of Equation 9 is true, then the original Equation 9 is also true. However, if Σᵢ₌₁ⁿ(bᵢ [symbol] dᵢ) becomes too large, it is possible to incorrectly show that Equation 9 is false. In order to minimize the chance this occurs, each bᵢ [symbol] dᵢ should be made as small as possible. To use a purposefully extreme example, one should not use an “Expected/Catastrophic” risk to remove an “Improbable/Negligible” risk. Instead, one should pick risks for {b} that are as close to, but only slightly greater, than the risks for {d} as possible; e.g., to pick the best possible examples, one can, for instance, use {b}={(occasional, critical)} to remove {d}={(remote, critical)}, or use {b}={(frequent, minor)} to remove {b}={(occasional, minor)}.

To illustrate the above in practice, recall that, if {b} represents one or more risks, {d} represents one or more risks, and {b} [symbol] {d}, then {a, b} [symbol] {c, d} implies the simpler equation {a} [symbol] {c}. Turning back to Equation 20, shown in FIG. 4Q), there is a risk count of “3” in the “Frequent-Serious/Major” cell on the left-hand side (specifically, cell 492) and a risk count of “3” in the “Frequent-Minor” cell on the right-hand side (specifically, cell 416). Since the probabilities are the same and each “Serious/Major” risk is only one level larger than each “Minor” risk, and since the larger risks are on the left-hand side of Equation 20, then the conditions for “Removing Unequal Risks” are met and Equation 20 can be simplified to the equation shown in FIG. 4R. Specifically, a risk count of “3” has been removed from both cell 492 and corresponding cell 416. As can be seen from FIG. 4R, there is a risk count of “2” in the “Probable-Serious/Major” cell on the left-hand side (specifically, cell 426) and a risk count of “1” in the “Probable-Minor” cell on the right-hand side (specifically, cell 424), plus a risk count of “1” in the “Probable-Negligible” cell on the right-hand side (specifically, cell 420). Since the probabilities are the same and a “Serious/Major” risk is larger than either a “Minor” risk or a “Negligible” risk, and since the larger risks are on the left-hand side of Equation 20, then the conditions for applying the “Removing Unequal Risks” rule are met and Equation 20 can further be simplified to the equation shown in FIG. 4S.

The “Moving Similar Risks” rule and “Moving Repeated risks” rule can now be used for a final simplification. First, the “Moving Similar Risks” rule can be used to move the risks in the “Remote—Critical” cell (cell 454) of the left-hand side of the equation shown in FIG. 4S, along the “5” risk band, to the “Probable-Minor” cell (cell 422). This simplification produces the equation shown in FIG. 4T.

Second, the “Moving Repeated Risks” rule can be used to move the “Probable-Minor” risk (cell 422) down one row to cell 492, as shown in the equation shown in FIG. 4U.

Finally, because the ten “Occasional-Minor” risks (cell 492) on the left-hand side of the above equation are each larger than the one “Occasional-Negligible” (cell 432), the two “Remote-Minor” (cell 448), and the one “Improbable-Minor” (cell 468) risks on the right-hand side, then all of the conditions are met to apply the “Removing Unequal Risks” rule and the equation in FIG. 4U can be simplified to the equation shown in FIG. 4V. Specifically, risk counts of “1,” “2,” and “1” are removed from cells 432, 448, and 468, respectively, while these same counts are removed from cell 492.

The equation shown in FIG. 4V can then be simplified to Equation 21, shown in FIG. 4W. Since the right-hand side 404 of Equation 21 has no risks and the left-hand side 402 of Equation 21 has twenty-one risks, Equation 21 is true. Since the risk algebra simplifications will not change whether the original equation (in this case, Equation 10) is true or false, then the fact that Equation 21 is true means that Equation 10 is also true. This then shows that the four risk algebra principles described above can be used to simplify Equation 10 until it becomes obvious whether the equation is true.

The creation and simplification of the equations described herein (e.g., Equation 9) can be, in at least one embodiment of the disclosure, critically reviewed from start to end by one or more individuals responsible for assessing benefit and risk (e.g., risk management professionals, other professionals, members of a cross-functional team, and the like). The review can additionally be documented.

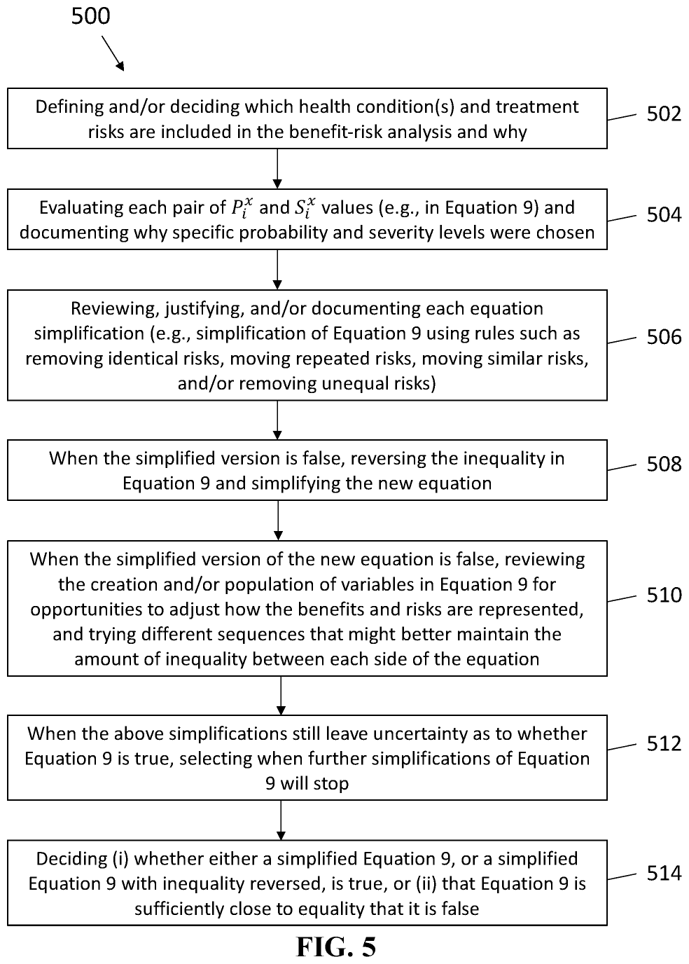

Turning now to FIG. 5, since there are different ways to create and simplify the above-mentioned equations, including, for instance, Equation 9 (shown in FIG. 2), a method 500 for reviewing one or more equations that define benefit and risk comprises, at block 502, defining and/or deciding which health condition(s) and healthcare action risks are included in the benefit-risk analysis and why. The method 500 can further comprise, at block 504, evaluating each pair of Pᵢ^{x} and Sᵢ^{x} values in Equation 9 and documenting why specific probability and severity levels were chosen. The method 500 can further comprise, at block 506, reviewing, justifying, and/or documenting each simplification. As described above herein, simplifications can follow one or more of the following four risk algebra rules: removing identical risks, moving repeated risks, moving similar risks, and removing unequal risks.

The removal of identical risks may often be a straightforward operation; documentation of applications of such removal may also be similarly simple (e.g., listing each pair of identical ordered pairs (Pᵢ^{x}, Sᵢ^{x}) that are removed from each side of a given equation (e.g., Equation 9).

The moving of repeated risks can have, in at least one example, several options within it. Accordingly, review, analysis, and/or documentation may include one or more explanations of which variation and/or option was used and which ordered pairs (Pᵢ^{x}, Sᵢ^{x}) were effected.

The moving of similar risks can be documented by, for instance, one or more explanations of steps used to ensure accurate implementation, as described in the “Moving of Similar Risks” section above herein.

The removing of unequal risks can be documented by, for instance, one or more explanations of steps used to ensure accurate implementation, as described in the “Removing of Unequal Risks” section above herein.

If, after simplifications, the simplified versions of the equations (e.g., simplified versions of Equation 9) is true, then the equations themselves (e.g., Equation 9) are true. However, if the simplified versions are false, then, before concluding that Equation 9 is false, the method 500 may further comprise, at block 508, reversing the inequality in Equation 9 and simplifying the new equation. If the simplified version of this new equation is false, then Equation 9 is false.

However, it is also possible that the simplified version of this new equation will be false again. This would indicate that Equation 9 is roughly equal and the method 500 may further comprise, at block 510, trying different sequences that might better maintain the amount of inequality between each side of Equation 9 (e.g., a different sequence that minimizes Et (bᵢ [symbol] dᵢ) more than the current sequence of simplification) or even reviewing the creation and/or population of variables in Equation 9 for any improvements that might break the near tie in Equation 9.

It should be acknowledged that there will be instances where Equation 9 is so close to equality that none of these techniques will be able to resolve for certain whether Equation 9 is true or not. In such a case, the method 500 may further comprise, at block 512, selecting when the simplification of Equation 9 will stop. The method 500 may further comprise, at block 514, deciding whether either a simplified Equation 9, or a simplified Equation 9 with the inequality reversed, is true or not, or deciding that Equation 9 is sufficiently close to an equality that Equation 9 is false.

### The Evolution of Risk Management

Applications of the benefit-risk analysis methods described herein can be understood with respect to the context of the evolution of risk management.

In the 1990's and before, there were no comprehensive, generally-accepted methods for assessing risks due to the provision of products and/or services, including, for instance, the risks presented by healthcare actions. Accordingly, the need for risk mitigations were left to the discretion and judgement of individuals and/or corporations charged with developing, implementing, and/or providing products and/or services (e.g., professionals charged with developing, implementing, and/or providing healthcare actions).

In the 2000's, ISO 14971 provided the first comprehensive, generally-accepted method to manage the risks presented by healthcare actions, including the need for risk mitigations. However, the responsibility for performing this analysis largely remained with engineers. In the 2010's, medical personnel began engaging with various portions of ISO 14971; however, engineers generally continued to complete the majority of risk management processes. Finally, in the 2020's, patient harms began to be analyzed and the role of medical personnel was often expanded to include non-local effects.

Two trends have emerged from the above developments: First, risk management has generally become more formal and detailed over time. Second, in the context of healthcare actions, the quantity of work performed by medical personnel is increasing. The method in this document continues both of these trends.

As the role of medical personnel continues to expand, it should be noted that professionals other than physicians can capably provide additional valuable expertise and resources, including sales and marketing personnel, nursing staff, clinicians and clinical researchers, and other medical technical workers. For instance, sales and marketing personnel often have intimate knowledge of both common practices around a particular healthcare action and the relevant scientific and/or medical literature and practices surrounding the healthcare action, including patients' health concerns. Accordingly, these people may be able to augment physicians by capably assisting in the characterization of patients' health risks. Further, nursing staff, including those that specialize in specific patient health concerns, can also contribute significantly in the characterization of patients' health risks.

Embodiments of the disclosure, which establishes the benefit of a healthcare action (e.g., a medical device and/or medical procedure) by looking at the change in the patient population's health concerns before and after a healthcare action, enable different populations of individuals, including one or more of the above types of professionals and/or workers, to provide valuable perspectives of patients' health concerns in the context of objective methods and systems.

### Food and Drug Administration (FDA) Approvals

Submissions to the U.S. Food and Drug Administration (FDA) to obtain Section 510 (k) clearance to market a healthcare action (e.g., a medical device) are based on a company's ability to show equivalence of the product they want to market with a predicate device that was already on market in 1976 and whose efficacy (i.e., “benefit”) and safety (i.e., “risk”) have been shown over time to be acceptable.

Pre-market authorizations (“PMA” or “PMAs”) are used to obtain clearance to market a device or product that was introduced since 1976. For this reason, their efficacy and safety record is considered insufficient to gain approval to market based on the safety record of other PMA products. For PMA products, a benefit-risk analysis may be key to showing that the product can be marketed.

Many products are based primarily on pre-1976 product technology, but have added some new feature(s) that did not exist prior to 1976. These product submissions for clearance to market the product should be a blend of the methods used for Section 510 (k) clearance and PMAs.

Embodiments of the present disclosure can therefore be used for benefit-risk analysis supporting one or more types of submissions to the FDA and/or other similar regulatory agencies to obtain clearance for marketing healthcare actions.

### ISO 14971

ISO 14971 defines the concept of “acceptable” and “unacceptable” risks based on a risk policy that is approved. ISO 14971 further establishes various criteria for risk acceptability.

Embodiments of the present disclosure can therefore be used for benefit-risk analysis with respect to “unacceptable” risks.

### Dominant Risks

Although the equations described above herein (e.g., Equation 9, shown in FIG. 2) allows for a large number of risks to be considered, and while this can be necessary to show whether benefit exceeds risk for healthcare actions (e.g., products and/or services such as, for instance, medical devices and/or medical procedures) when the amount of benefit and risk are nearly equal, Equation 9 can be quite simple if a few risks dominate the other risks and the risk difference between the two sides of the equation exceeds the size of those other risks. In such a situation, Equation 9 can be simplified, in at least one embodiment of the disclosure, to only use a few pre-determined risks that are dominant. In at least one example, Equation 9 can also be accompanied by one or more justifications for omitting the other risks.

In at least another example, Equation 9 can have several large risks on the left-hand side (e.g., R_{B}) that a given healthcare action reduces to small risks on the right-hand side (e.g., R_{A}). This may result from, for instance, a highly-effective medical therapy. Thus, it may become apparent from the benefit-risk analyses described herein that a few of the large risks on the left-hand side are far larger than all of the risks on the right-hand side (e.g., R_{B}∪R).

If this is the case, then the documentation of the benefit-risk analysis can use such dominant risks to establish a maximum bound for all of the risks on the right-hand side of Equation 9. Accordingly, it may be true that benefit exceeds risk because all of the risks on the right-hand side of the equation are smaller than this maximum bound. In at least one example, risk algebra rules as described above herein can be used to represent the aforementioned (e.g., by combining the removal of repeated risks with the removal of unequal risks).

Selecting a Therapy from a List of Alternative Therapies

In at least one embodiment of the present disclosure, a method for selecting a therapy (e.g., a best therapy) from a list of alternative therapies is disclosed. Equation 4 can be used for selecting such a best therapy.

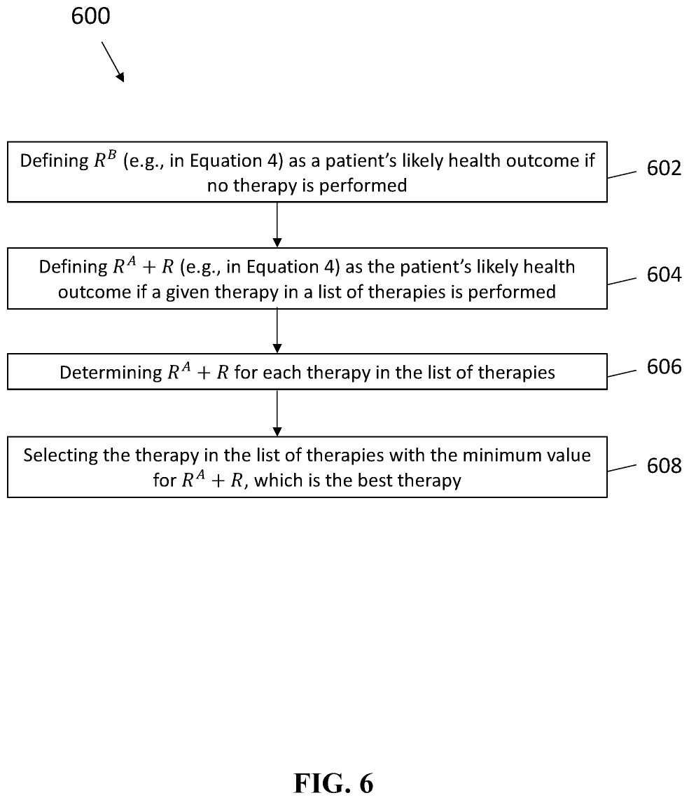

Turning now to FIG. 6, a method 600 is shown for selecting a best therapy from a list of therapies. The method 600 comprises, at block 602, defining R^{B} (e.g., in Equation 4) as a patient's likely health outcome if nothing (e.g., no therapy) is done. The method 600 can further comprise, at block 604, defining R^{A}+R (e.g., in Equation 4) as the patient's likely health outcome if a given therapy is performed. Selecting the best therapy from a list of alternative therapies can then be achieved by, at block 606, determining R^{A}+R for each therapy in a list of therapies, and then, at block 608, selecting the therapy with the minimum value for R^{A}+R. The above assumes that Equation 9 (shown in FIG. 2) is met for each alternative therapy.

### Customizing Benefit-Risk Analysis to Small Populations or Individuals

In at least one embodiment of the present disclosure, a method to customize the benefit-risk analysis to an individual patient is disclosed. Generally, weights can be used to customize a benefit-risk analysis to individual patients. Just as benefit-risk analysis was shown above herein to facilitate selecting the best therapy from among alternative therapies, customizing the analysis to an individual patient's preferences can be invaluable to facilitate holistic discussions with a patient about healthcare action options and ensuring all factors are considered, with no specific factors given excessive weight.

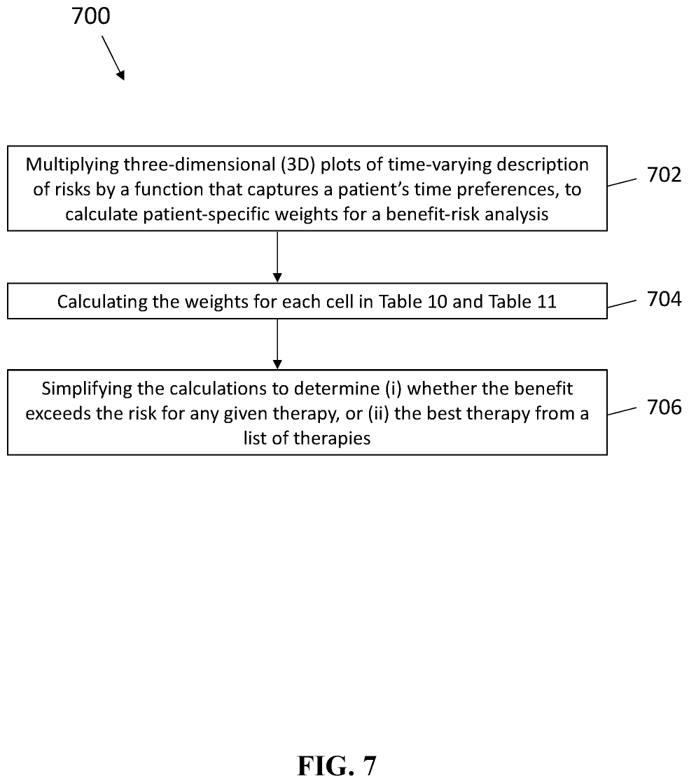

Turning now to FIG. 7, a method 700 is shown for customizing a benefit-risk analysis to an individual patient based on the patient's preferences. These preferences may be, in at least one example, time-based. That is, a patient might, for example, prefer to maximize their quality of life during one or more upcoming life-events (e.g., a birth or marriage) at the expense of quality of life after those life-events. The method 700 can comprise, at block 702, multiplying three-dimensional (3D) plots of time-varying description of risks by a function that captures the patient's time preferences. The integration of a point-by-point product of two functions is a standard operation in calculus, which is referred to as a “convolution integral.”

To extend the above example, if a patient cares only about their health for the next two years, the patient's time-preference is simply a “box function” that is one unit high for the next two years and zero for all other time points. Convoluting this “box function” with the 3D plot for each risk will provide a weight for each risk that is customized to the patient's preferences.

Once patient-specific weights are calculated, the method 700 further comprises, at block 704, calculating the weights for each cell in Tables 10 and 11. The method further comprises, at block 706, simplifying the calculations to determine whether the benefit exceeds the risk (for any given therapy) or determine the best therapy from a list of alternative therapies (e.g., with reference to FIG. 6).

### Accounting for Time-Varying Benefits and Risks

ISO 14971 defines “risk” as a combination of “severity” and “probability,” while the FDA defines both “risk” and “benefit” as a combination of “severity,” “probability,” and “duration.” At least one embodiment of the disclosure provides a systematic framework for severity, probability, and duration.

Benefit-risk analyses based on severity and probability have been described above herein. With respect to “duration,” at least one embodiment of the disclosure comprises a method to account for the effect of time on risk. “Time” is an important, and usually under-accounted for, factor in risk management. While the duration of a risk is mentioned briefly in various risk management systems (e.g., as documented in Table 2 above herein), any accounting for time using these systems becomes muddled because the same table is also used to describe severities, which are independent of time. In addition, some healthcare actions (e.g., some medical devices and/or medical procedures) require a significant amount of time before they produce benefits, and many such benefits may wear out and/or diminish over time.

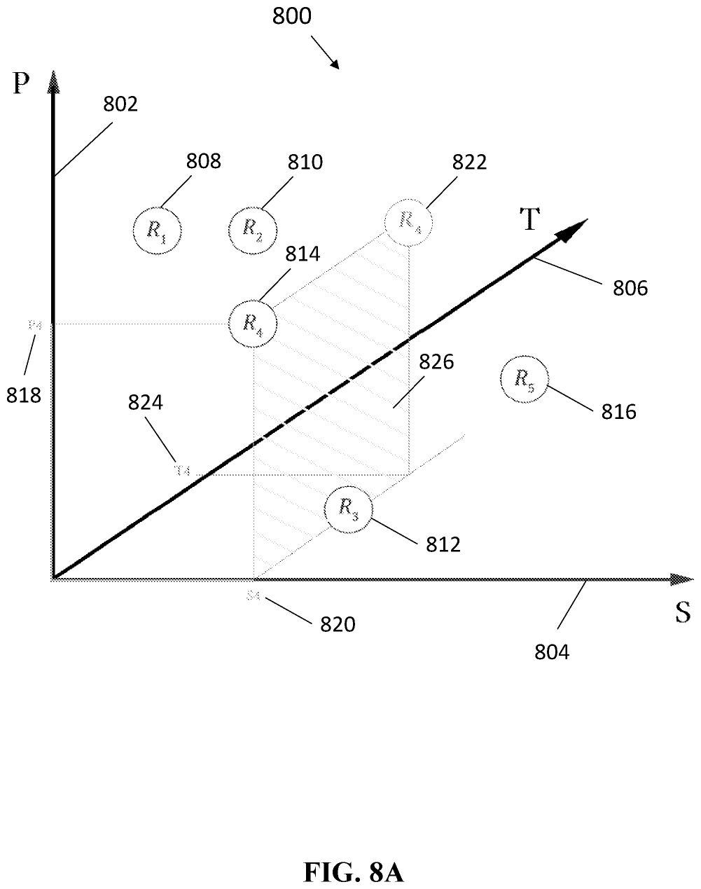

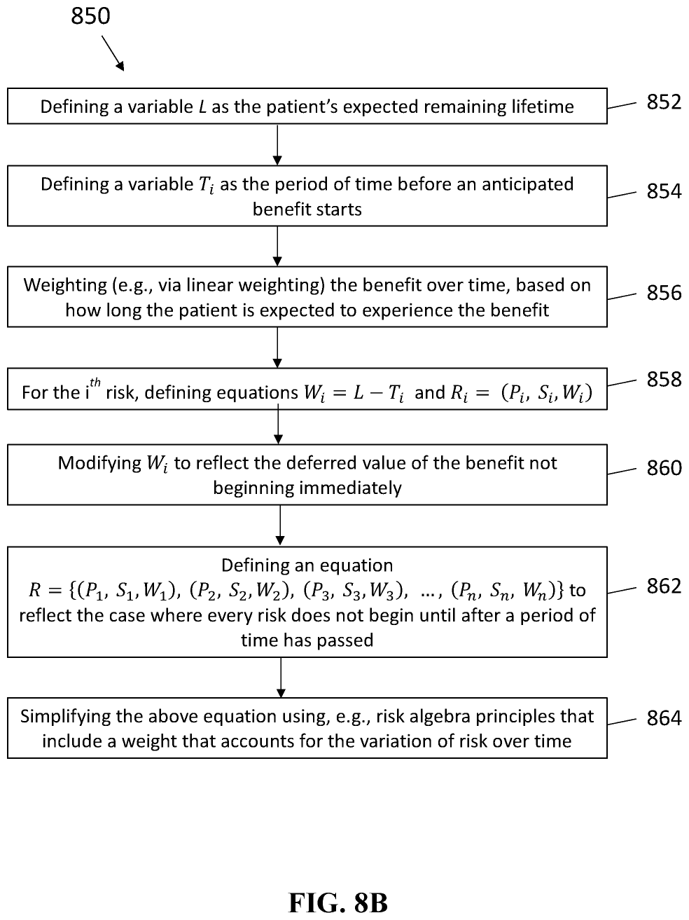

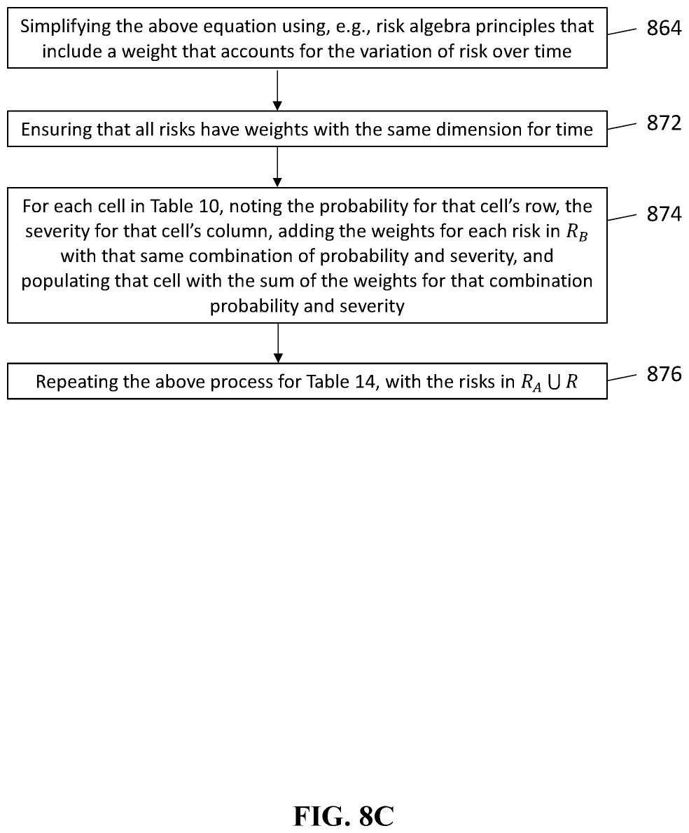

Turning now to FIG. 8A, a three-dimensional (3D) plot 800 of probability P, severity S, and time T is shown. Specifically, probability P is shown on y-axis 802, severity S is shown on x-axis 804, and time T is shown on z-axis 806. Various risks R are shown (specifically, R₁ 808, R₂ 810, R₃ 812, R₄ 814, and R₅ 816, each of which are, as described above herein, combinations of a specific probability P and a severity S. Thus, R₄ 814 is the combination of P₄ 818 and S₄ 820. To account for the changes in R₄ over time, R₄ can be extended along axis 806 to a point 822 at a time T₄ 824. The area 826 bounded by these axes therefore represents the weight based on time, W, given to a particular benefit or risk (in this example, R₄) over time.

The area 826 can be generalized to more complex shapes, such as, for instance, where both the probability and severity are functions of time and the principles of integration, from calculus, are needed to determine the area (e.g., for two dimensions, time and either probability or severity) or volume (e.g., for three dimensions, probability, severity, and time).

In at least one embodiment of the disclosure, area or volume are used as the weight for each risk to account for time. That is, the i^{th} risk, Rᵢ, is represented by the ordered triplet:

$${R}_{i} = ( {P}_{i} , {S}_{i} , {W}_{i} ) \qquad ( Equation 22 )$$

The equation for R then becomes:

$$R = \{ ( {P}_{1} , {S}_{1} , {W}_{1} ) , ( {P}_{2} , {S}_{2} , {W}_{2} ) , ( {P}_{3} , {S}_{3} , {W}_{5} ) , … , ( {P}_{n} , {S}_{n} , {W}_{n} ) \} \qquad ( Equation 23 )$$

Time-dependent weights can be added to each risk in different ways. A non-limiting example is shown in FIG. 8B. In at least one embodiment of the disclosure, a method 850 of modeling a benefit that does not begin until after a period of time has passed and then continues, unchanged, for the rest of the patient's life comprises, at block 852, defining a variable L as the patient's expected remaining lifetime. The method 850 further comprises, at block 854, defining a variable Tᵢ as the period of time before the benefit starts. The method 850 further comprises, at block 856, weighting (e.g., via linear weighting) the benefit over time, based on how long the patient is expected to experience those benefits. The method 850 further comprises, at block 858, for the i^{th} risk, defining equations Wᵢ=L−Tᵢ and Rᵢ=(Pᵢ, Sᵢ, L−Tᵢ). The method 850 may further comprise, at block 860, modifying Wᵢ to reflect the deferred value of the benefit not beginning immediately. Such modification may, in at least one example, account for patient preference, as described further herein.

The method 850 may further comprise, at block 862, defining an equation:

$$R = \{ ( {P}_{1} , {S}_{1} , L - {T}_{1} ) , ( {P}_{2} , {S}_{2} , L - {T}_{2} ) , ( {P}_{3} , {S}_{3} , L - {T}_{3} ) , … , ( {P}_{n} , {S}_{n} , L - {T}_{n} ) \} ,$$

which is a version of Equation 23 in the case where every risk does not begin until after a period of time has passed. In at least other examples, each value of Wᵢ can be calculated by a different equation.

The method 850 may further comprise, at block 864, simplifying the equation using, for instance, risk algebra principles (e.g., as described above herein) that include a weight that accounts for the variation of risk over time.

FIG. 8C shows further steps in the aforementioned simplification. First, at block 872, ensure that all of the risks have weights with the same dimension for time. Then, at block 874, for each cell in a table showing the probability and severity of risks (e.g., the below Table 10), note the probability for that cell's row, the severity for that cell's column, add the weights for each risk in R_{B} with that same combination of probability and severity, and populate that cell with the sum of the weights for that combination probability and severity. Then, at block 876, repeat the process set forth in block 874 for Table 14, with the risks in R_{A} ∪R. This is equivalent to what was described for risks without a weight for time if Wᵢ=1 is assigned for all values of i.

TABLE 10
R_{B} Table Showing Combinations of Probability and Severity

| | | | Serious/ | | Catastrophic/ |
|---|---|---|---|---|---|
| R_{B} | Negligible | Minor | Major | Critical | Fatal |
| Expected | | | | | |
| Often | | | | | |
| Frequent | | | | | |
| Probable | | | | | |
| Occasional | | | | | |
| Remote | | | | | |
| Improbable | | | | | |

The risk algebra principles and/or rules described herein function when risk is represented by a triplet (e.g., Equation 22) or as an ordered pair (e.g., Equation 3). In at least one example, simplifying risks (e.g., with the “Removing Identical Risks” rule) may result in the term remaining, with some amount of time associated with the Probability-Severity combination. The rule of “Removing Unequal Risks” is therefore useful for resolving such Probability-Severity combinations with small amounts of time remaining.

### Uncertainty in the Benefit-Risk Analysis

Since the inputs to benefit-risk analysis embodiments described herein have uncertainty levels surrounding them, the analysis also has an uncertainty interval around its conclusion that benefit outweighs risk. Knowing this interval can inform decisions about how much the analysis decision can be trusted. For instance, if the uncertainty interval of each Table in Equation 9 is much smaller than the difference between the right and left sides, then the conclusion of the benefit-risk analysis may be more trustworthy. Conversely, if the uncertainty interval of each Table in Equation 9 s much greater than the difference between the right and left sides, then the benefit-risk analysis may be further modified.

In at least one embodiment of the disclosure, the Taylor Series can be used to approximate variation in the inputs to any one or more of the equations described above herein. Specifically:

$${σ}_{R}^{2} \approx {\sum }_{i = 1}^{k} {( \frac{\partial R}{\partial {x}_{i}} )}^{2} {σ}_{{x}_{i}}^{2} .$$

As mentioned previously herein, the table look-up operation (e.g., for Equation 9) can be implemented as a table look-up with wide ranges for each value. Accordingly, the respective derivatives,

$$\frac{\partial R}{\partial {S}_{i}} and \frac{\partial R}{\partial {P}_{i}} ,$$

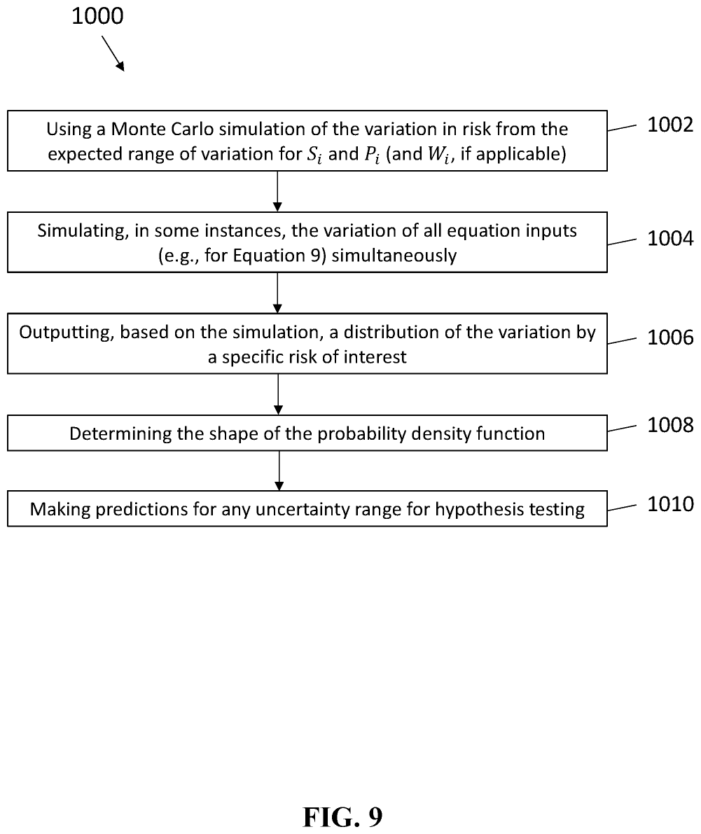

may not be continuous functions. Turning now to FIG. 9, a method 1000 is shown for estimating the uncertainty associated with a benefit-risk analysis. The method 1000 comprises, at block 1002, using a Monte Carlo simulation of the variation in risk from the expected range of variation for Sᵢ and Pᵢ (and Wᵢ, if applicable). The method 1000 may further comprise, at block 1004, simulating, in some instances, the variation of all equation inputs (e.g., for Equation 9) simultaneously. Once the modeling of the variation of all the inputs to the equation is complete, a Monte Carlo simulation with, for instance, a hundred randomly chosen values from each input will, when these hundred sets of inputs are run through the equation, provide a reliable distribution of the variation by the risk of interest. The method 1000 can therefore further comprise, at block 1006, outputting, based on the simulation, a distribution of the variation by a specific risk of interest. Once the variation has been determined, the shape of the probability density function can be determined, as shown at block 1008. Finally, the method 1000 may comprise, at block 1010, making predictions for any uncertainty range for hypothesis testing.

### Different Patient Populations

In at least one embodiment of the disclosure, the benefit and/or risk of a particular healthcare action (e.g., a particular medical device and/or medical procedure) can be calculated for different patient populations using one or more of the methods described above herein. Special care should be taken with patient populations to ensure that the risks are based on data unique to these populations and ensure that any risks that are unique to, or absent from, this patient population are accounted for in the model of patient health. By noting the specific patient populations or sub-populations in which the benefits outweigh the risks, the range of populations that can use a healthcare action can be appropriately limited or increased.

Multiple Conditions and/or Diseases

In at least one embodiment of the disclosure, if a particular healthcare action (e.g., a particular medical device and/or medical procedure) can be, and/or is intended to be, used to treat multiple conditions and/or diseases, each such condition and/or disease may have its own benefit-risk calculation and/or analysis using one or more of the methods described above herein. Thus, the healthcare action being evaluated could, for instance, only be indicated for use for those conditions and/or diseases (or for those combinations of conditions and/or diseases) where the benefit exceeds the risk.

### Instructions for Use (IFU)

Generally, the instructions for use (IFU) of a particular healthcare action (e.g., a particular medical device and/or medical procedure) should define how that healthcare action is indicated for use. Thus, in at least one embodiment of the disclosure, the benefit-risk analysis can comprise showing the benefit to exceed the risk for one or more, or each, of the different indications for use. Such a process can reveal that different indications for use have different benefit-risk ratios. The calculated benefit-risk ratios for each indication can assist in defining when the healthcare action should be used despite the risks, and for which patient population or sub-population.

### Customizing Risk Metrics for Emotional Risks

Most healthcare actions treat a patient's physical harms; however, currently-used methods of evaluating benefits and risk do not function well in the context of emotional benefits and/or emotional risks (e.g., whether the benefits of a surgery whose goal is to improve someone's appearance outweigh the risks of that surgery). The benefit-risk analysis described herein can be used for such emotional benefits and/or emotional risks.

As mentioned above herein, Table 2 and Table 3 are non-limiting examples of the tables that can be used to quantify the probability and severity of harm. Part of the flaws present in currently-used methods of evaluating benefits and risk is that purely aesthetic healthcare actions (e.g., purely aesthetic medical devices and/or medical procedures) are not “fixing” a specific physical injury that can be measured by the aforementioned tables. Instead, the primary benefit of applying these healthcare actions is the improved self-esteem of patients.

As discussed above herein, benefits and risks are traditionally stated in terms that make comparison difficult (e.g., the previously cited FDA guidance on Benefit-Risk Analysis). This difficulty is especially evident for a purely aesthetic healthcare action whose benefit lies in the mismatch of a purely emotional and/or mental benefit versus risks that are both physical and emotional/mental.

In order to account properly for benefits to the patient's improved self-esteem and self-image, at least one embodiment of the disclosure accounts for emotional and/or mental risks instead of only physical risks. In order that such emotional and/or mental risks are given proper weight, relative to any physical risks, a severity table can be constructed based on a wider variety of healthcare actions than only purely aesthetic healthcare actions. For instance, non-aesthetic healthcare actions (e.g., non-aesthetic medical devices and/or medical procedures) with mental benefits and both mental and physical risks should be used, together with the benefits and risks of purely aesthetic healthcare actions, to create more-appropriate probability and severity metrics than are in, for instance, ISO 14971.

Non-aesthetic healthcare actions with emotional and/or mental benefits and both mental and physical risks include various healthcare actions (including drugs) to treat mental disorders ranging from anxiety to schizophrenia to manic depression. At least one embodiment of the disclosure uses non-aesthetic healthcare actions (e.g., for mental disorders) along with purely aesthetic healthcare actions (e.g., creams and surgeries) to construct one or more severity tables (e.g., three- and/or five-level severity tables) to create risk metrics for healthcare actions that benefit a patient's emotional and/or mental state.

### Models of Patient Health

In at least one embodiment, a model for patient health is disclosed. The methods described herein enable the measurement of the amount of benefit and risk from a given healthcare action (e.g., a given medical device and/or medical procedure) significantly more objectively than is currently possible. These benefits and risks therefore create a significantly more objective measure of the health of a patient population than is currently possible.

Once built, this model can be used to compare the rate of various risks to a given patient population or sub-population of a given healthcare action. Changes to the patient's health can come from the healthcare action's intended action, from a side effect therein, or from the patient population's underlying health concerns (e.g., the changes may be unrelated to the healthcare action). In at least an additional example, the patient health model can be extended to include these factors using Bayesian statistics. For instance, comorbidity factors can be included for the patient population and their likely impact on the health of the patient population.

One advantage of extending the model to include such factors is that it enables reliable comparisons of predicted and actual rates of occurrence. Such comparisons can be especially useful with respect to occasional harmful events (e.g., a heart attack or allergic reaction). If the model is accurate with respect to known diseases and effects, and if the rates of occasional harmful events are different from the actual rate by statistically significant amounts (which may be determined by calculating one or more uncertainty intervals as described herein), then the difference can be attributed to the healthcare action under consideration. Otherwise, the side effect can be presumed to be due to random chance, with no causal connection to the healthcare action.

In at least an additional example, the calculated rate of side effects is accompanied by a calculated confidence interval.

In at least a further example, the model can be extended to include multiple disease states. The model may also include, for instance, the comorbidity factors discussed above. For example, if D represents the set of disease states of patients treated with a healthcare action, then l=n (D) refers to the number of disease states treated with the healthcare action, S represents the severity rating of the j^{th} harm of the i^{th} disease state, mᵢ=n (Sⱼⁱ) represents the number of harms caused by the i^{th} disease state, Pⱼⁱ is the probability rating of the j^{th} harm occurring at the severity Sⱼⁱ for the i^{th} disease state, Wⱼⁱ is a time factor (e.g., as discussed above herein) for the j^{th} harm of the i^{th} disease state, and R is the likely amount of risk to the patient's health from the healthcare action. As a non-limiting example, if a user uses a 7×5 severity table, then

$${P}_{j}^{i} ∈ \{ \begin{matrix}Expected , Often , Frequent , Probable , \\ Occasional , Remote , Improbable\end{matrix} \} and {S}_{j}^{i} ∈ \{ \begin{matrix}Negligible , Minor , Serious / Major , \\ Critical , Catastrophic / Fatal\end{matrix} \} .$$

With these assumptions, the risk to the patient from the healthcare action is:

$$R = ( \begin{matrix}( {P}_{1}^{1} , {S}_{1}^{1} , {W}_{1}^{1} ) , ( {P}_{2}^{1} , {S}_{2}^{1} , {W}_{2}^{1} ) , \\ ( {P}_{3}^{1} , {S}_{3}^{1} , {W}_{3}^{1} ) , … , ( {P}_{{m}_{1}}^{1} , {S}_{{m}_{1}}^{1} , {W}_{{m}_{1}}^{1} ) , \\ ( {P}_{1}^{2} , {S}_{1}^{2} , {W}_{1}^{2} ) , ( {P}_{2}^{2} , {S}_{2}^{2} , {W}_{2}^{2} ) , \\ ( {P}_{3}^{2} , {S}_{3}^{2} , {W}_{3}^{2} ) , … , ( {P}_{{m}_{2}}^{2} , {S}_{{m}_{2}}^{2} , {W}_{{m}_{2}}^{2} ) , \\ ( {P}_{1}^{3} , {S}_{1}^{3} , {W}_{1}^{3} ) , ( {P}_{2}^{3} , {S}_{2}^{3} , {W}_{2}^{3} ) , \\ ( {P}_{3}^{3} , {S}_{3}^{3} , {W}_{3}^{3} ) , … , ( {P}_{{m}_{3}}^{3} , {S}_{{m}_{3}}^{3} , {W}_{{m}_{3}}^{3} ) , … , \\ ( {P}_{1}^{1} , {S}_{1}^{1} , {W}_{1}^{1} ) , ( {P}_{2}^{1} , {S}_{2}^{1} , {W}_{2}^{1} ) , \\ ( {P}_{3}^{1} , {S}_{3}^{1} , {W}_{3}^{1} ) , … , ( {P}_{l}^{1} , {S}_{l}^{1} , {W}_{l}^{1} ) ,\end{matrix} ) \qquad ( Equation 34 )$$

Similar to Equation 34, risk models for both R_{A} and R_{B} can be built to represent complete models of patient health. In at least a further example, the model considers synergistic effects from multiple exposures with the healthcare action. See also ISO 31010, Section 6.3.5.1.

In at least one example, the model is generated by determining the typical characteristics of a patient suffering from one or more diseases or conditions that the healthcare action under consideration is designed to treat. Such a determination could be made by identifying one or more diseases or conditions the healthcare action is intended to treat, and estimating, using medical data (e.g., published data, including, for instance clinical data) regarding the disease characteristics, the patient's likely state of health. Such an estimate may include, for example, determining how the disease will likely harm the patient, the probability of such harm occurring, the duration of harm, and whether the harm will occur immediately or at some point in the future.

Benefit-Risk Analysis for Product Compliance and/or Product Availability

Given the central role that benefit-risk analysis has in evaluating products, and before releasing new products to market, it should be no surprise that benefit-risk analysis can also be a key indicator of product compliance (e.g., compliance with applicable regulations) and/or product availability (e.g., whether field actions are required).

Currently-used tables for benefit-risk analysis are only relative; that is, their absolute value has no meaning. By contrast, the benefit-risk analysis methods described herein compare measures of risk with measures of benefit, which provides a meaningful absolute value. A company or regulator can, for instance, review the benefit-risk analysis to determine if post-market circumstances have changed the original (i.e., pre-market) analysis significantly.

Looking at changes to the benefit-risk analysis can be useful when assessing the criticality of audit observations. For instance, if a production line is cited for compliance problems because it was not sufficiently clean, the risks to users from a dirty production line can be increased to reflect the audit finding. If increasing these risks changes the product's risk profile sufficiently that the product's risks exceed the benefits, then the non-compliant stock could be withdrawn from the field. Conversely, if increasing these risks to users as far as possible does not change the fact that the product's benefits outweigh its risks, then field action may not need to be taken.

Often, an important consideration is whether a benefit, or anticipated benefit, can be achieved through the use of alternative solutions that do not have a specific risk, or have a smaller risk. See ISO 14971, Section 7.4.1. Therefore, it is possible that, although an audit observation or combination of observations does not increase risk to a user sufficiently that a given product's risks exceed the benefits, those same observation(s) may increase the risk sufficiently that the increase in risk is too high given the list of alternative products or possibilities, as discussed above herein with respect to selecting a best therapy from a list of alternative therapies. In such a situation, non-compliant stock could therefore still be withdrawn from the field, although such withdrawal would be due to a different healthcare action (e.g., a different product and/or service such as, for instance, a different medical device and/or medical procedure) providing a better therapy and/or a better outcome, and not because the original healthcare action's risks exceeds its benefits.

### Computer Device Implementations

One or more of the examples described herein can be implemented on one or more computing systems, as described in further detail below.

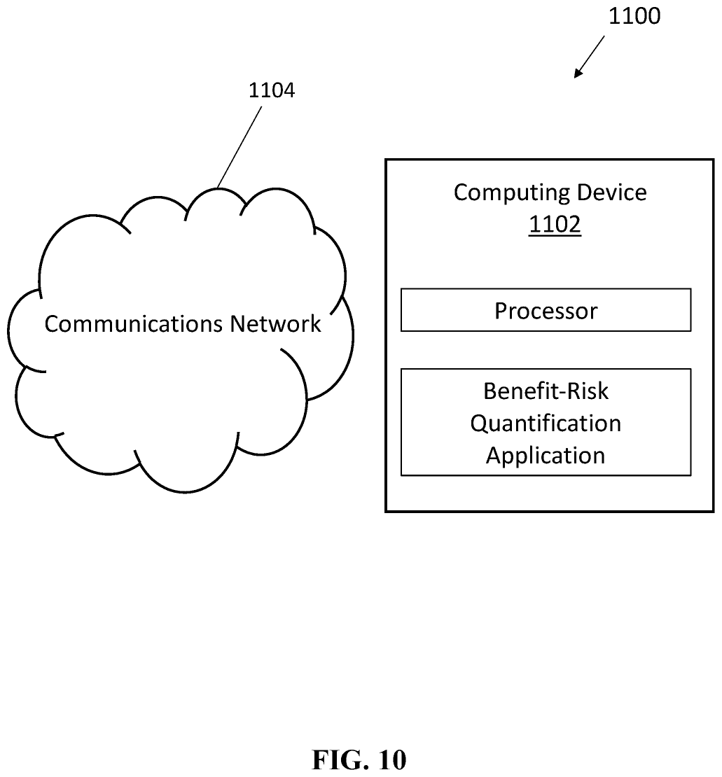

FIG. 10 is a block diagram of a computing system 1100 for identifying and/or calculating benefits of a healthcare action (e.g., a product and/or service such as, for instance, a medical device and/or medical procedure), and for comparing the benefits with the risks, according to an example embodiment. Thus, the computing system 1100 may perform, for instance, any of the steps and/or calculations described above herein.

The system 1100 comprises one or more computing devices 1102 that may execute one or more applications to identify and/or calculate benefits (e.g., of a healthcare action), and/or to compare the benefits with the risks. Additionally, the applications can be used to send information to, or receive information from, a specific user or users. The applications can further be capable of scheduled or triggered communications or commands when various events occur (e.g., when one or more calculations results in an error, when more information is needed defining the types of risks, and the like).

The one or more computing devices 1102 can be used to store acquired computational data, as well as other data in memory and/or a database. The memory may be communicatively coupled to one or more hardware processing devices.

The one or more computing devices 1102 may further be connected to a communications network 1104, which can be the Internet, an intranet, or another wired or wireless communication network. For example, the communication network 1104 may include a Mobile Communications (GSM) network, a code division multiple access (CDMA) network, 3^{rd} Generation Partnership Project (GPP) network, an Internet Protocol (IP) network, a wireless application protocol (WAP) network, a Wi-Fi network, a satellite communications network, or an IEEE 802.11 standards network, as well as various communications thereof. Other conventional and/or later developed wired and wireless networks may also be used.

The one or more computing devices 1102 include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions related to the benefit-risk quantification application. In addition, the one or more computing devices 1102 further include at least one communications interface to transmit and receive communications, messages, and/or signals.

Thus, information processed by the one or more computing devices 1102, or the applications executed thereon, may be sent to another computing device, such as a remote computing device, via the communication network 1104.

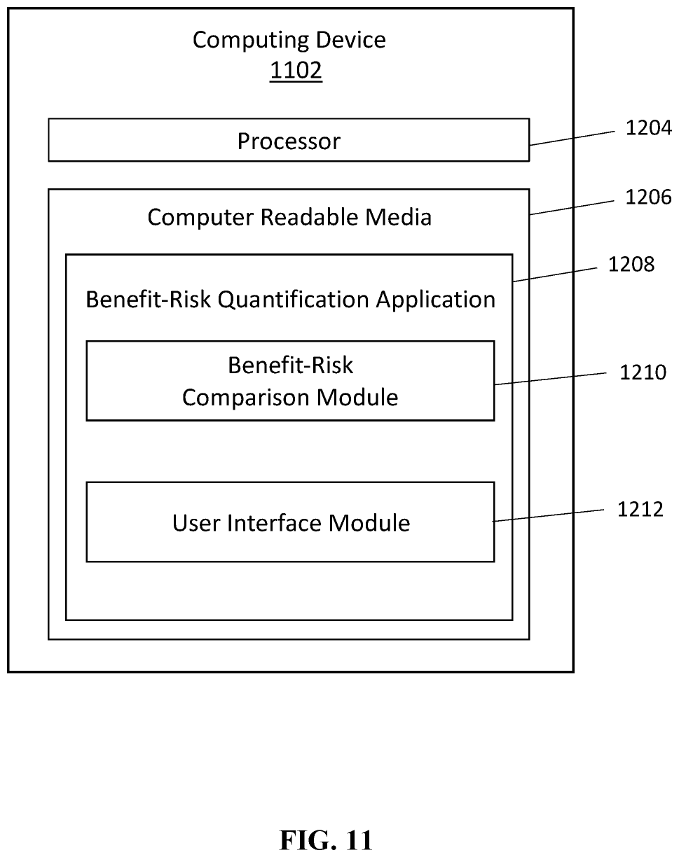

FIG. 11 illustrates a block diagram of a computing device 1102 according to an example embodiment. The computing device 1102 includes computer readable media (CRM) 1206 in memory on which a benefit-risk quantification application 1208 or other user interface or application is stored. The computer readable media may include volatile media, nonvolatile media, removable media, non-removable media, and/or another available medium that can be accessed by the processor 1204. By way of example and not limitation, the computer readable media comprises computer storage media and communication media. Computer storage media includes non-transitory storage memory, volatile media, nonvolatile media, removable media, and/or non-removable media implemented in a method or technology for storage of information, such as computer/machine-readable/executable instructions, data structures, program modules, or other data. Communication media may embody computer/machine-readable/executable instructions, data structures, program modules, or other data and include an information delivery media or system, both of which are hardware.

The benefit-risk quantification application 1208 can include a benefit-risk comparison module 1210 that is operable to perform various functions, such as, for instance, identifying specific benefits and/or risks of a healthcare action (e.g., a medical device and/or medical procedure), calculating values for such benefits and/or risks, and comparing the calculated benefits to the calculated risks. The module 1210 may also be operable to obtain data from other sources, such as a user, a database, and the like, and to process that data to correct for imperfect, inaccurate, or absent calculations and/or models regarding the quantification of benefits, risks, and benefit-risk ratios.

Using a local high-speed network, the computing device 1102 may receive the aforementioned data in near real time, process the data, and generate calculations. These calculations may be executed by one or more algorithms within the benefit-risk quantification application 908 or other stored applications.

Measured or calculated data may be monitored to generate an event and an alert if something is out of range (e.g., one or more calculations and/or equations is incorrect or throws an error, one or more additional data points or definitions is needed, etc.). As mentioned herein, an alert may also be sent after one or more benefit-risk decisions has been made. Such alerts may be sent in real-time or near real-time using an existing uplink or dedicated link. The alerts may be sent using email, SMS, push notification, or using an online messaging platform to end users and computing devices, among others.

The benefit-risk quantification application 1208 may provide data visualization using a user interface module 1212 for displaying a user interface on a display device. As an example, the user interface module 1212 generates a native and/or web-based graphical user interface (GUI) that accepts input and provides output viewed by users of the computing device 1102. The computing device 1102 may provide real-time automatically and dynamically refreshed information on the identification and/or quantification of one or more types of benefits and/or risks. The user interface module 1212 may send data to other modules of the benefit-risk quantification application 1208 of the computing device 1102, and retrieve data from other modules of the benefit-risk quantification application of the computing device 1102 asynchronously without interfering with the display and behavior of the user interface displayed by the computing device 1102.

Accordingly, embodiments of the present disclosure, by providing methods to identify and/or calculate benefits and/or risks, improve the rate at which a benefit-risk decision can be made. Embodiments also simplify benefit-risk decision-making in situations where multiple possible decisions must be made to see which decision has the greatest difference between benefit and risk.

Thus, embodiments of the disclosure can be used to, for instance, calculate the benefits and risks of a healthcare action (e.g., a medical device and/or medical procedure) using the same scale, and compare the calculated benefits and risks. Measuring both benefits and risks with the same metric makes the task of comparing the two more straightforward, thereby improving reliability of the decision on whether benefits or risks are greater.

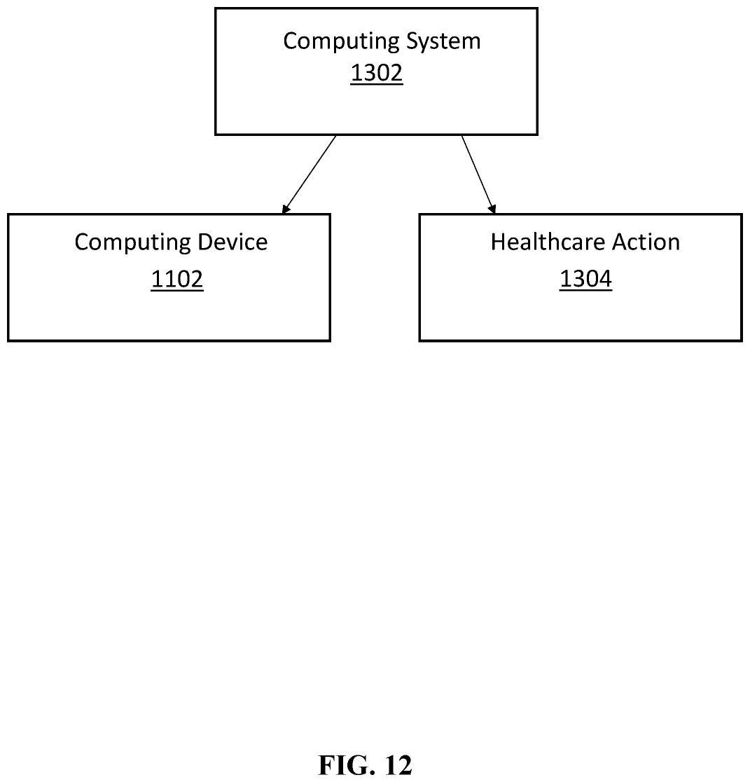

Further, one or more computing systems can implement one or more aspects of the technology and/or methods described herein. FIG. 12 shows an example of such a computing system 1302, which may include one or more computing devices (e.g., computing device 1102) and/or processing units, which include one or more processors and software. The one or more computing devices (e.g., computing device 1102) may execute one or more applications to operate one or more applications, such as, for example, the benefit-risk quantification application 1208 described above herein, or one or more portions thereof. The computing system 1102 may further control, monitor, and/or extract data from, for instance, a healthcare action 1304 (e.g., a medical device and/or medical procedure). The computing system can further comprise a graphical user interface (GUI) so that a user may control the system or portions thereof, such as, for instance, the healthcare action 1304.

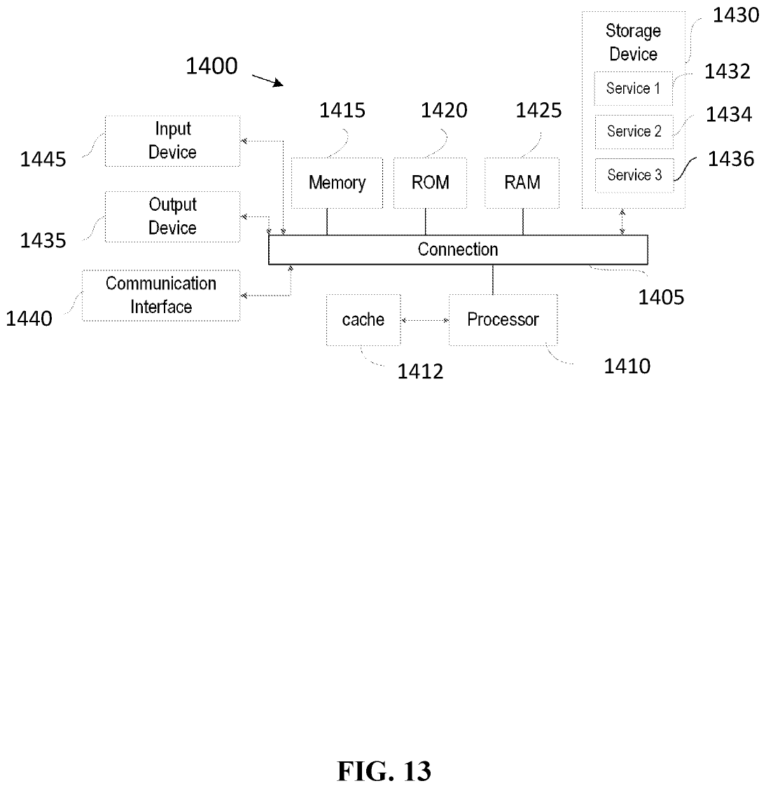

FIG. 13 shows an example of computing system 1400, which can be for example any computing device such as the computing device 1102, or any component thereof in which the components of the system are in communication with each other using connection 1405. Connection 1405 can be a physical connection via a bus, or a direct connection into processor 1410, such as in a chipset architecture. Connection 1405 can also be a virtual connection, networked connection, or logical connection.

In some embodiments, computing system 1400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 1400 includes at least one processing unit (CPU or processor) 1410 and connection 1405 that couples various system components including system memory 1415, such as read-only memory (ROM) 1420 and random access memory (RAM) 1425 to processor 1410.

Computing system 1400 can include a cache of high-speed memory 1412 connected directly with, in close proximity to, or integrated as part of processor 1410.

Processor 1410 can include any general purpose processor and a hardware service or software service, such as services 1432, 1434, and 1436 stored in storage device 1430, configured to control processor 1410 as well as a special-purpose processor where software instructions are incorporated into the actual processor design. Processor 1410 may essentially be a completely self-contained computing system, containing multiple cores or processors, a bus, memory controller, cache, etc. A multi-core processor may be symmetric or asymmetric.

To enable user interaction, computing system 1400 includes an input device 1445, which can represent any number of input mechanisms, such as a microphone for speech, a touch-sensitive screen for gesture or graphical input, keyboard, mouse, motion input, speech, etc. Computing system 1400 can also include output device 1435, which can be one or more of a number of output mechanisms known to those of skill in the art. In some instances, multimodal systems can enable a user to provide multiple types of input/output to communicate with computing system 1400. Computing system 1400 can include communications interface 1440, which can generally govern and manage the user input and system output. There is no restriction on operating on any particular hardware arrangement, and therefore the basic features here may easily be substituted for improved hardware or firmware arrangements as they are developed.

Storage device 1430 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 1430 can include software services, servers, services, etc., that when the code that defines such software is executed by the processor 1410, it causes the system to perform a function. In some embodiments, a hardware service that performs a particular function can include the software component stored in a computer-readable medium in connection with the necessary hardware components, such as processor 1410, connection 1405, output device 1435, etc., to carry out the function.

For clarity of explanation, in some instances, the present technology may be presented as including individual functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the disclosures herein can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, Universal Serial Bus (USB) devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Embodiments of the present disclosure will be further understood by reference to the following non-limiting examples.

### EXAMPLES

The below examples set forth a benefit-risk analysis for a given healthcare action. Example 1 shows such an analysis for a first patient population, while Example 2 shows the analysis for a different patient population.

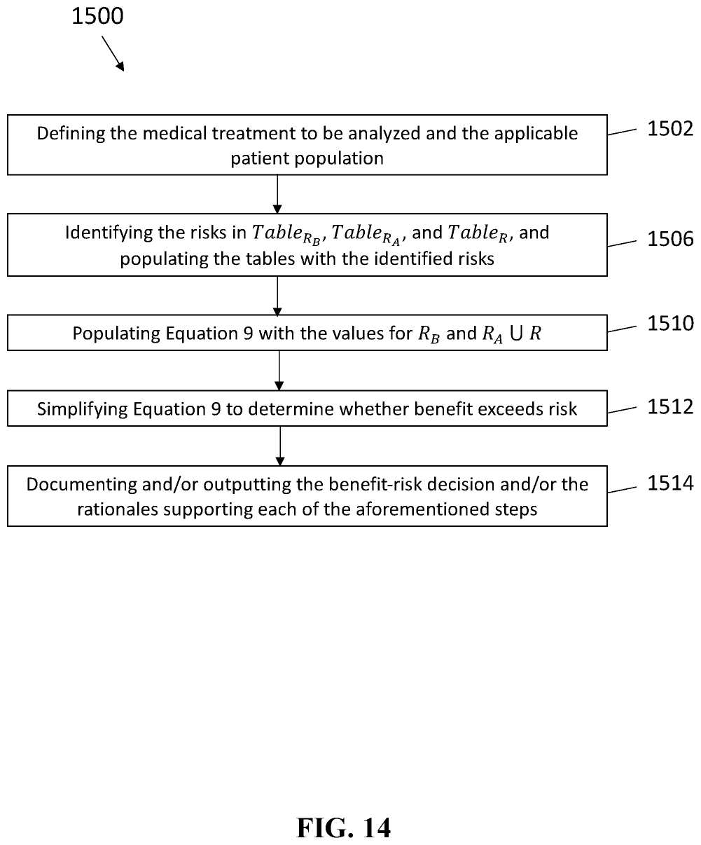

Generally, the below examples utilize the following method, shown in FIG. 14 for determining whether the healthcare action's benefits exceed its risks. First, the method 1500 generally comprises, at block 1502, defining the healthcare action and the applicable patient population. This may include, for instance, ensuring the healthcare action's options are broken down sufficiently that there is only one benefit-risk decision being analyzed, and/or ensuring that the patient population is sufficiently clear and/or limited, since risks generally vary significantly based on the patient population. The method 1500 further comprises, at block 1506, identifying the risks in Table_{R}_{_{B}}, Table_{R}_{_{A}}, and Table_{R}, as described below herein, and populating the tables with the identified risks. Such tables include definitions of R_{B}, R_{A}, and R, as those variables have been described above herein. The aforementioned identification may be achieved by, for instance, consulting different fields of expertise to ensure depth and/or breadth of knowledge with respect to the healthcare action and the risks of applying such a healthcare action to the patient population. The method 1500 further comprises, at block 1508, calculating, via the tables for R_{A} and R, R_{A} ∪R. The method 1500 further comprises, at block 1510, populating Equation 9 with the values for R_{B} and R_{A} ∪R. The method 1500 further comprises, at block 1512, simplifying Equation 9 to determine whether benefit exceeds risk. This can be done using, for instance, the risk algebra rules described above herein. The method 1500 may further comprise, at block 1514, documenting and/or outputting the benefit-risk decision and/or the rationales supporting each of the aforementioned steps.

### Example 1

In this example, with particular reference to block 1502, the healthcare action is a blood transfusion and the applicable patient population is patients in the United States who have sufficient upper gastrointestinal (GI) bleeding to meet emergency room (ER) criteria for needing such a transfusion.

The purpose and risks of performing such blood transfusions to these patients may be identified by reference to knowledge of ER medicine, physicians specializing in ER medicine, knowledge of medical devices (e.g., apheresis equipment) for collecting and infusing blood, and the like.

With particular reference to block 1506, the risks in Table_{R}_{_{B}}, Table_{R}_{_{A}}, and Table_{R} can be identified. Table_{R}_{_{B}} can be generated by identifying the health conditions that might cause this patient population to receive a blood transfusion (e.g., health conditions that might cause a bleeding ulcer to lose enough blood that a transfusion might be indicated to treat the blood loss). An example of this shown in Table 11 below.

TABLE 11
Sample Health Conditions for Blood Transfusions

| Medical Description of | |
|---|---|
| the Health Condition | Non-Specialist Description |
| Altered Mental State | The patient becomes disoriented, more |
| | easily distracted, and less able to focus. |
| Circulatory Collapse | The patient has bled so much that the |
| | heart has too little blood volume to |
| | pump. Standard of care directs that such |
| | a patient has an endoscopy after the |
| | transfusion to identify and/or correct the |
| | reason for such extensive bleeding. |
| Heart attack, stroke, | The circulatory system cannot transport |
| Kidney injury | sufficient oxygen to sustain the body's |
| | organs. |
| Esophageal Varices | A pre-existing risk factor that can result |
| | in significant upper GI bleeding. |
| | Variceal bleeding generally accounts |
| | for 10-30% of all cases of upper |
| | gastrointestinal bleeding. |

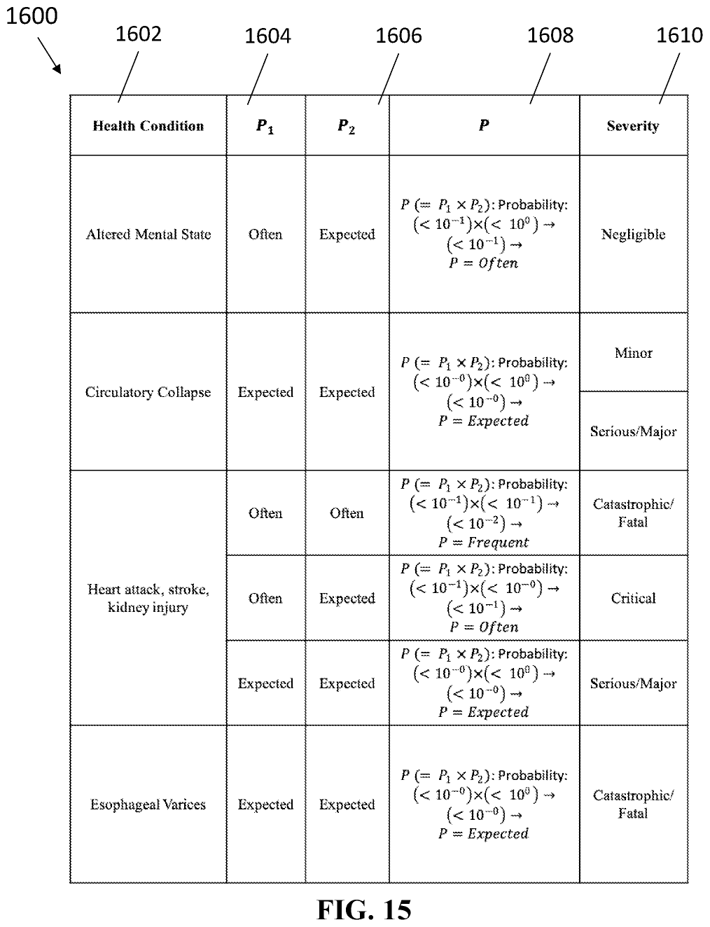

Based on the specific health condition, probability and severity categories can be identified and assigned, as shown in FIG. 15. Specifically, table 1600 has five columns, columns 1602, 1604, 1606, 1608, and 1610. Column 1602 shows the various health conditions, specifically, in this example, altered mental state; circulatory collapse; heart attack, stroke, and kidney injury; and esophageal varices. Column 1604 shows P₁, or probability, for each of these conditions. Column 1606 shows P₂, or probability and severity, for each of these conditions. Column 1608 shows P for each of the listed conditions, with P (=P₁×P₂). Column 1610 shows the severity for each of the listed conditions.

P₁ can be obtained from various sources of information, including, for instance, reference to emergency room medicine resources and/or physicians. For example, hemorrhagic shock may be relevant to the altered mental state condition. Such shock can be defined as, for instance, (1) volume loss from 30% to 40% of total blood volume, from 1500 mL to 2000 mL, (2) a significant drop in blood pressure and changes in mental status, (3) heart rate and respiratory rate elevation (e.g., more than 120 beats per minute (BPM)), (4) urine output decline, and/or (5) capillary refill delays. With respect to circulatory collapse, studies have indicated around a 5% incidence. With respect to esophageal varices, between 1966 and 2013, there have been about 50 cases of esophageal varices triggered by transfusions. According to the Red Cross, there are 29,000 transfusions per day, or 10,585,000 transfusions per year.

Similarly, P₂ can be obtained from various sources of information, including, for instance, reference to emergency room medicine resources and/or physicians.

Next, with particular reference to block 1506, each cell in the following table for R_{B} can be populated. To do so, note the severity and the probability from FIG. 15, and enter the number of risks with that cell's combination of severity and probability.

TABLE 12
Sample Table for R_{B}

| | | | Serious/ | | Catastrophic/ |
|---|---|---|---|---|---|
| R_{B} | Negligible | Minor | Major | Critical | Fatal |
| Expected | 0 | 1 | 2 | 0 | 1 |
| Often | 1 | 0 | 0 | 1 | 0 |
| Frequent | 0 | 0 | 0 | 0 | 1 |
| Probable | 0 | 0 | 0 | 0 | 0 |
| Occasional | 0 | 0 | 0 | 0 | 0 |
| Remote | 0 | 0 | 0 | 0 | 0 |
| Improbable | 0 | 0 | 0 | 0 | 0 |

Table_{R}_{_{A}} can be generated by identifying the health conditions in this patient population that might remain after receiving the healthcare action. Since B=R_{B}−R_{A}, Table_{R}, will normally contain the same health conditions as Table_{R}_{_{B}}, particularly since Table_{R} contains any health risks that were created by the healthcare action.

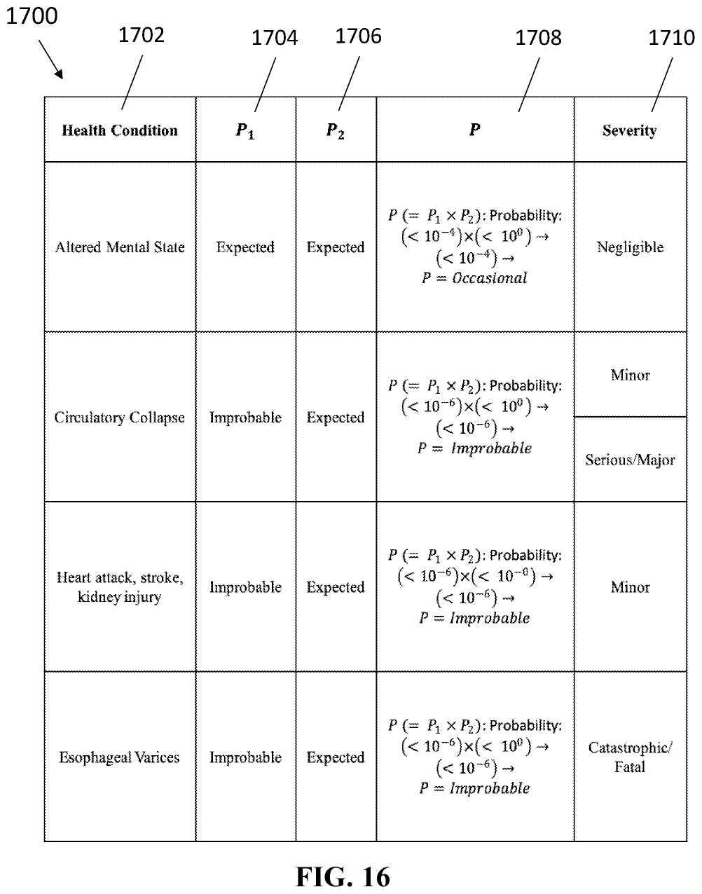

FIG. 16 shows a table 1700 for R_{A}. As with FIG. 15, table 1700 contains column 1702 for the various health conditions, column 1704 showing P₁, or probability, for each of these conditions, column 1706 showing P₂, or probability and severity, for each of these conditions, column 1708 showing P for each of the listed conditions, with P (=P₁×P₂), and column 1710 showing the severity for each of the listed conditions.

Next, with particular reference to block 1506, each cell in the following table for R_{A} can be populated. To do so, note the severity and the probability from FIG. 16, and enter the number of risks in the previous table with that cell's combination of severity and probability.

TABLE 13
Sample Table for R_{A}

| | | | Serious/ | | Catastrophic/ |
|---|---|---|---|---|---|
| R_{A} | Negligible | Minor | Major | Critical | Fatal |
| Expected | 1 | 0 | 0 | 0 | 0 |
| Often | 0 | 0 | 0 | 0 | 0 |
| Frequent | 0 | 0 | 0 | 0 | 0 |
| Probable | 0 | 0 | 0 | 0 | 0 |
| Occasional | 0 | 0 | 0 | 0 | 0 |
| Remote | 0 | 0 | 0 | 0 | 0 |
| Improbable | 0 | 2 | 1 | 0 | 1 |

Next, R can be populated by identifying the health conditions that, for this patient population, might remain after the blood transfusion, as shown in Table 14 below.

TABLE 14
Sample Health Conditions Remaining After A Blood Transfusion

| Medical Description of | |
|---|---|
| the Health Condition | Non-Specialist Description |
| Febrile Nonhemolytic | Febrile non-hemolytic transfusion reaction |
| Reaction | (FNHTR) is the most common type of |
| | transfusion reaction. It is a benign occurrence |
| | with symptoms that include fever but not |
| | directly related with hemolysis. It is caused |
| | by cytokine release from leukocytes within |
| | the donor product as a consequence of white |
| | blood cell breakdown. |
| Allergic reaction to | Typically, a rash, itching/swelling (especially |
| Tylenol or Benadryl | of the face/tongue/throat), severe dizziness, |
| | trouble breathing. |
| Acute hemolytic | An acute hemolytic transfusion reaction |
| transfusion reaction | (AHTR) is triggered by host antibodies |
| | destroying donor red blood cells. |
| Graft vs. host disease | If the person receiving the transfusion has a |
| | compromised immune system. |
| TACO (Transfusion | Fluid overload, similar to acute congestive |
| Associated | heart failure |
| Circulatory | |
| Overload) | |
| Transfusion-related | Antibodies get activated, causing breathing |
| acute lung injury | issues |
| Infection | Patient acquires an infection from the needle |
| | or donated blood |
| Hepatitis B | Patient acquires Hepatitis B from the donated |
| | blood |
| Hepatitis A | Patient acquires Hepatitis A from the donated |
| | blood |
| HIV | Patient acquires HIV from the donated blood |
| Mislabeled blood bag | The label on the blood bag is affixed to the |
| | wrong bag |

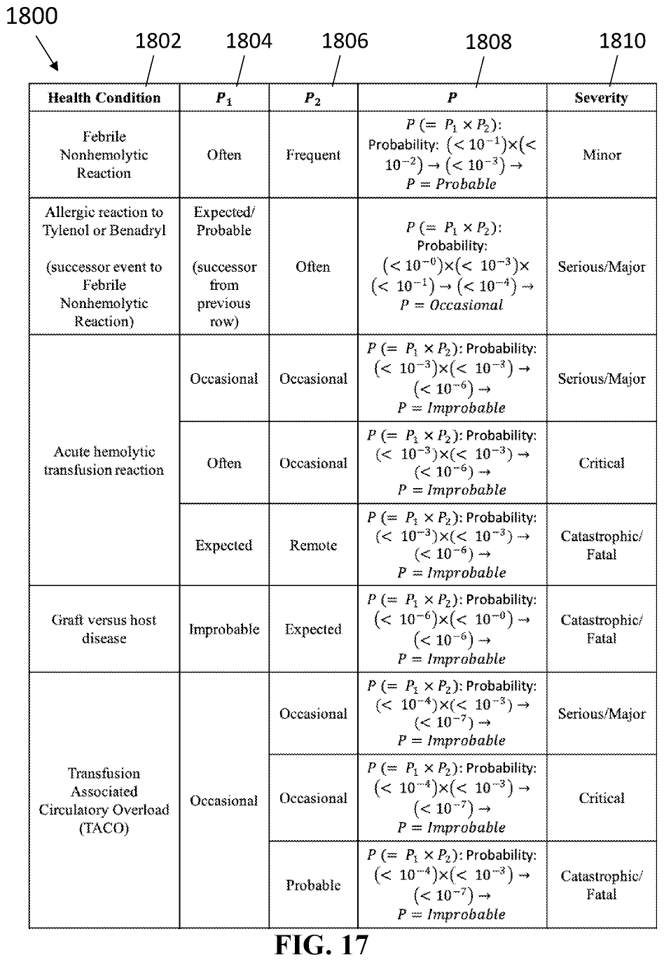

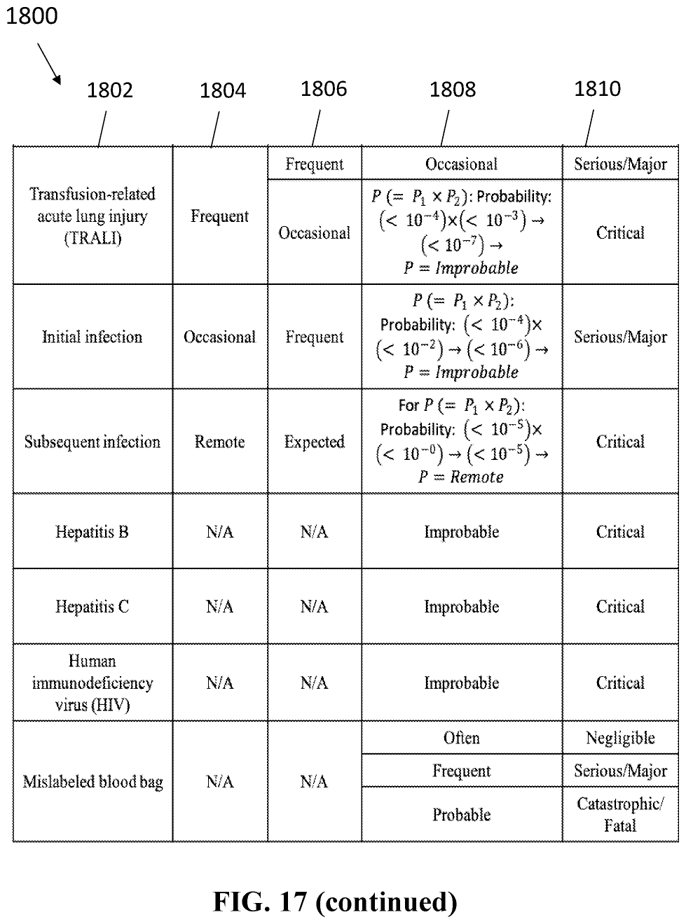

Based on the above health condition, probability and severity categories can be identified and assigned, as shown in FIG. 17. Specifically, table 1800 has five columns, columns 1802, 1804, 1806, 1808, and 1810. Column 1802 shows the various health conditions, specifically, in this example, febrile non-hemolytic reaction, allergic reaction subsequent to the febrile non-hemolytic reaction, acute hemolytic transfusion reaction, graft versus host disease, transfusion associated circulatory overload (TACO), transfusion-related acute lung injury (TRALI), initial infection, subsequent infection, hepatitis B, hepatitis C, human immunodeficiency virus (HIV), and a mislabeled blood bag. Column 1804 shows P₁, or probability, for each of these conditions. Column 1806 shows P₂, or probability and severity, for each of these conditions. Column 1808 shows P for each of the listed conditions, with P (=P₁×P₂). Column 1810 shows the severity for each of the listed conditions.

P₁ can be obtained from various sources of information, including, for instance, reference to emergency room medicine resources and/or physicians. For example, febrile nonhemolytic transfusion reactions (FNHTRs) can be commonly encountered transfusion reactions with an overall per unit rate of about 1% to about 3%. With respect to subsequent allergic reactions, Tylenol or Benadryl is administered after the patient receives a transfusion for FNHTRs. Reactions can occur in about 1.6% of all patients taking non-steroidal anti-inflammatory drugs (NSAIDs). With respect to acute hemolytic transfusion reactions, the rate may roughly be about 5 per 100,000. With respect to graft versus host disease, the frequency may be challenging to ascertain due to its relative rarity; some underlying risk factors include a prior bone marrow transplant. With respect to transfusion associated circulatory overload (TACO), the general frequency may be about 17.1 per 100,000. With respect to transfusion-related acute lung injury (TRALI), estimates have been 1 in 5,000 components, mostly in whole blood, 1 in 7,900 units of fresh frozen plasma, and 1 in 432 units of whole blood-derived platelet concentrates. With respect to initial infection, the incidence of any bacterial infection can be around 1 in 30,000. With respect to subsequent infection, the incidence of any bacteremia infection (subsequent to bacterial infection) can be around 1 in 500,000. With respect to hepatitis B, the incidence of acquiring hepatitis B from a blood transfusion ranges from about 1 in 1 million to 1 in 1.5 million. With respect to hepatitis C, the incidence of acquiring hepatitis C from a blood transfusion ranges from about 1 in 2 million to 1 in 2.6 million. With respect to HIV, the incidence of acquiring HIV from a blood transfusion is about less than 1 in 2 million. With respect to a mislabeled blood bag, actual harmful events due to errors can occur in about 0.26% of patients.

Similarly, P₂ can be obtained from various sources of information, including, for instance, reference to emergency room medicine resources and/or physicians.

Next, with particular reference to block 1506, each cell in the following table for R can be populated. To do so, note the severity and the probability from FIG. 17, and enter the number of risks with that cell's combination of severity and probability.

TABLE 15
Sample Table for R

| | | | Serious/ | | Catastrophic/ |
|---|---|---|---|---|---|
| R | Negligible | Minor | Major | Critical | Fatal |
| Expected | 0 | 0 | 0 | 0 | 0 |
| Often | 1 | 0 | 0 | 0 | 0 |
| Frequent | 0 | 0 | 1 | 0 | 0 |
| Probable | 0 | 1 | 0 | 0 | 1 |
| Occasional | 0 | 0 | 2 | 0 | 0 |
| Remote | 0 | 0 | 0 | 1 | 0 |
| Improbable | 0 | 0 | 3 | 6 | 3 |

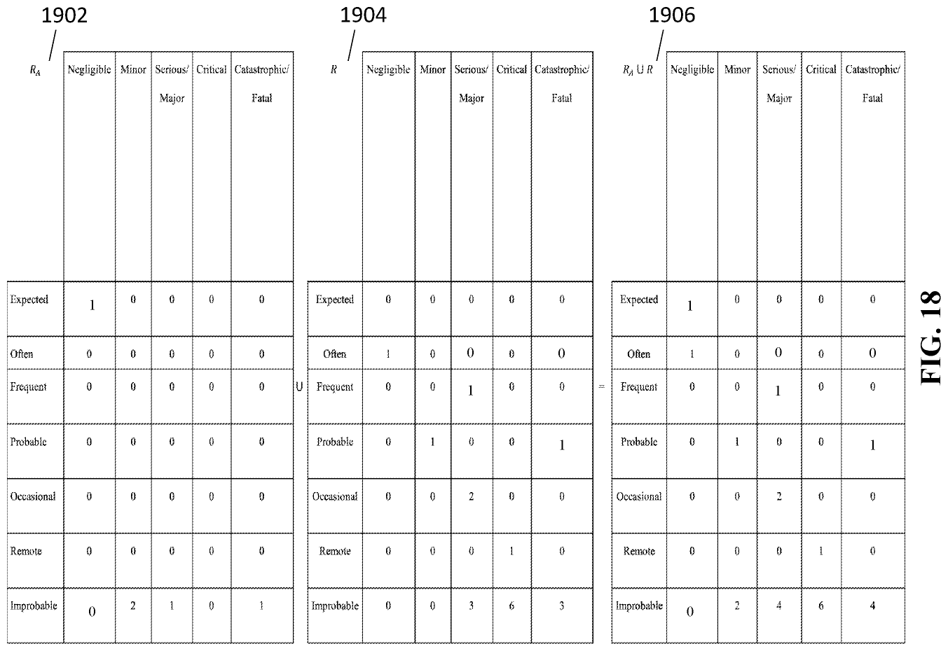

Then, with particular reference to blocks 1508 and 1510, R_{A} ∪R can be calculated and Equation 9 can be populated accordingly. Specifically, R_{A} ∪R can be calculated using R_{A} and R, and their respective tables above. This generates Equation 24, shown in FIG. 18. Specifically, tables 1902, 1904, and 1906 are shown for R_{A}, R, and R_{A} ∪R, respectively.

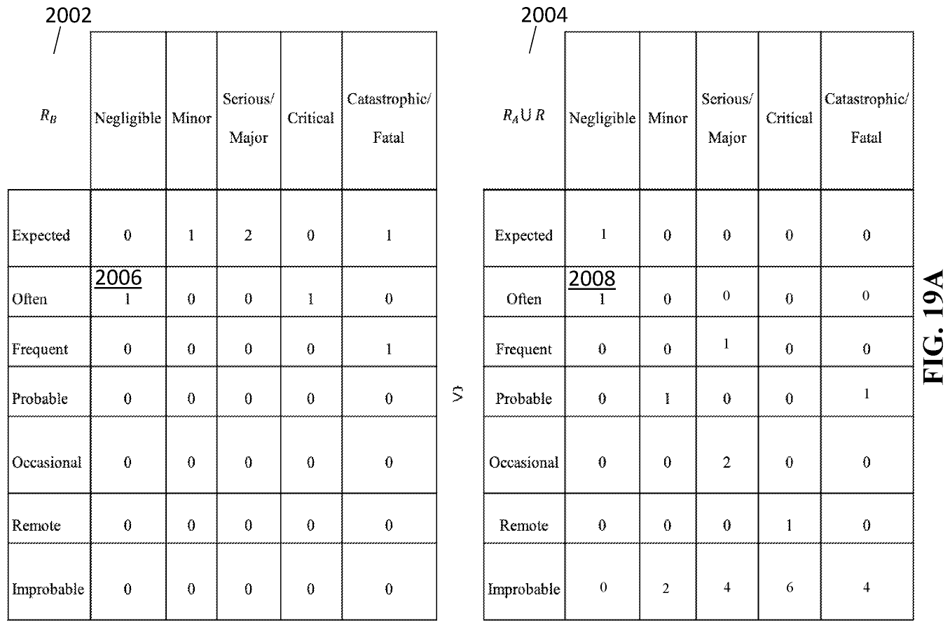

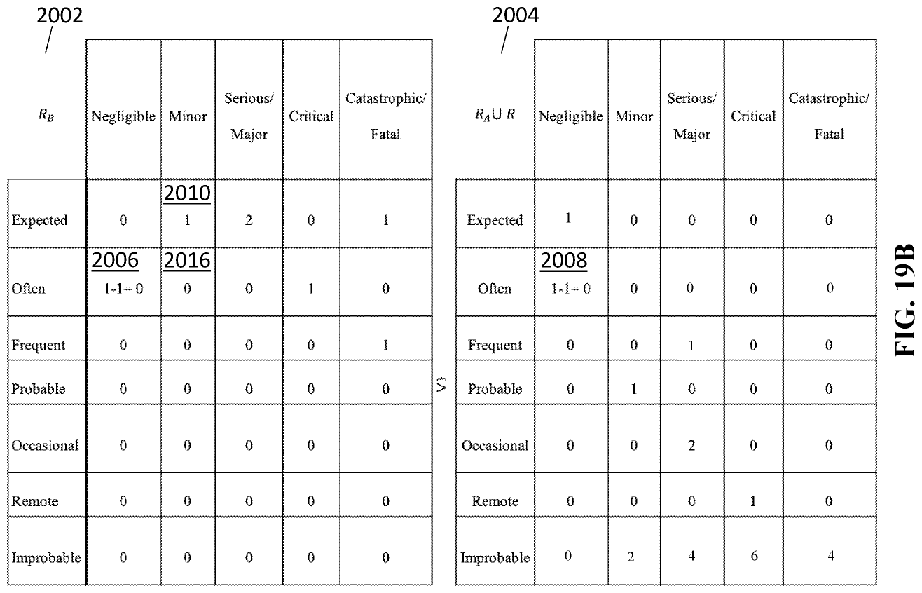

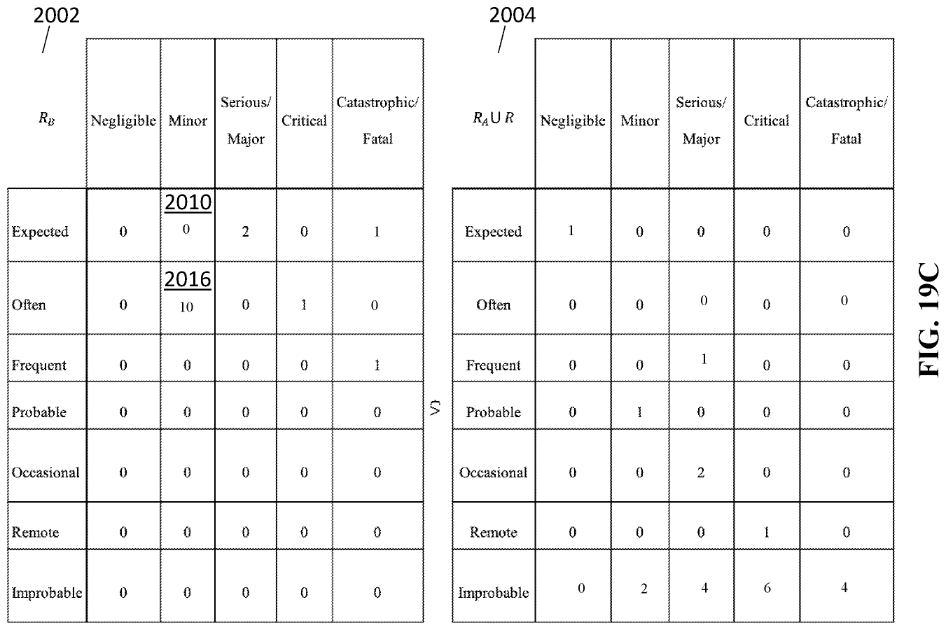

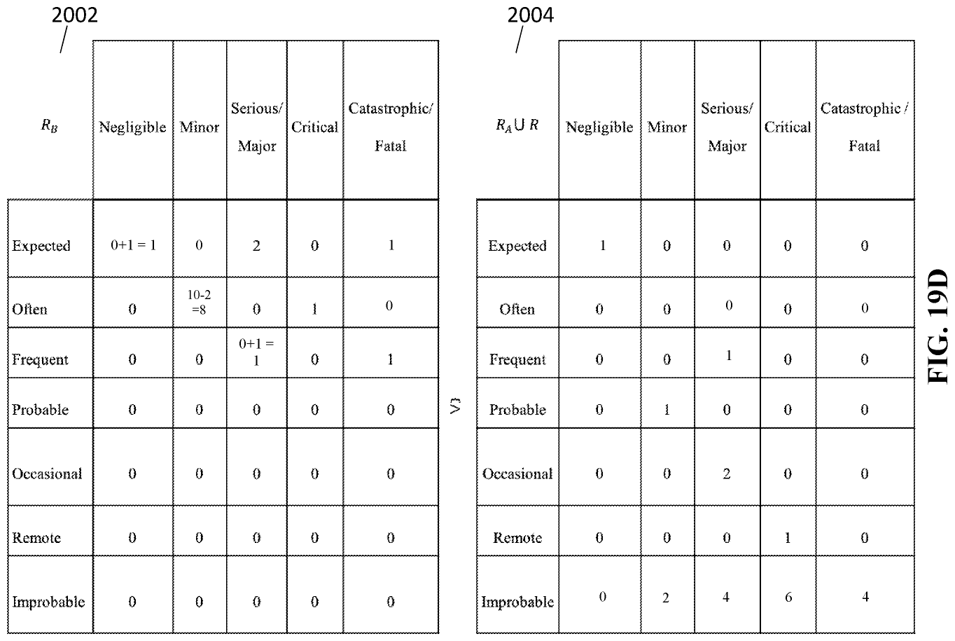

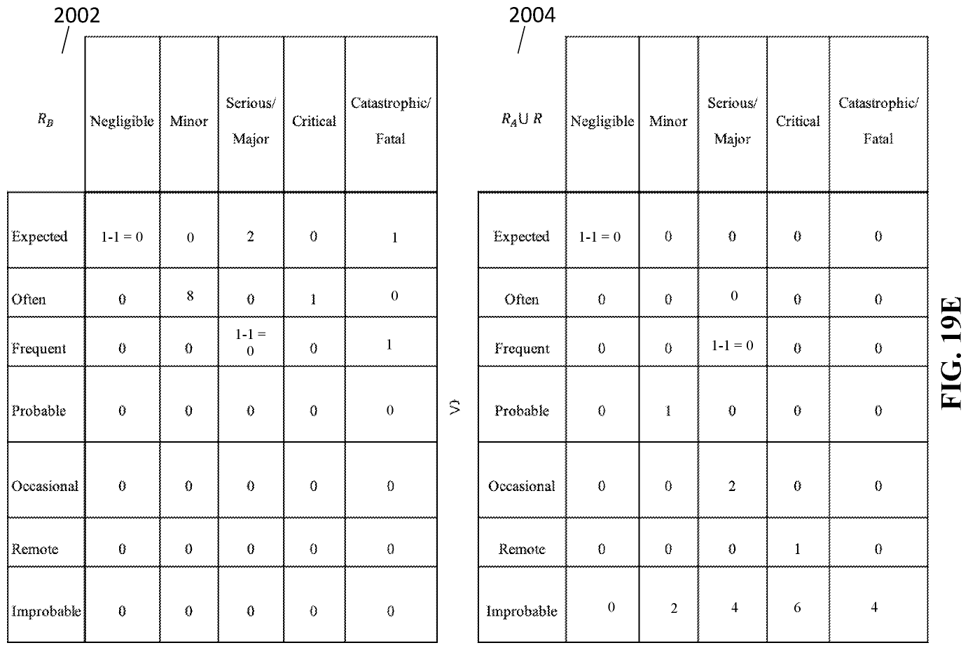

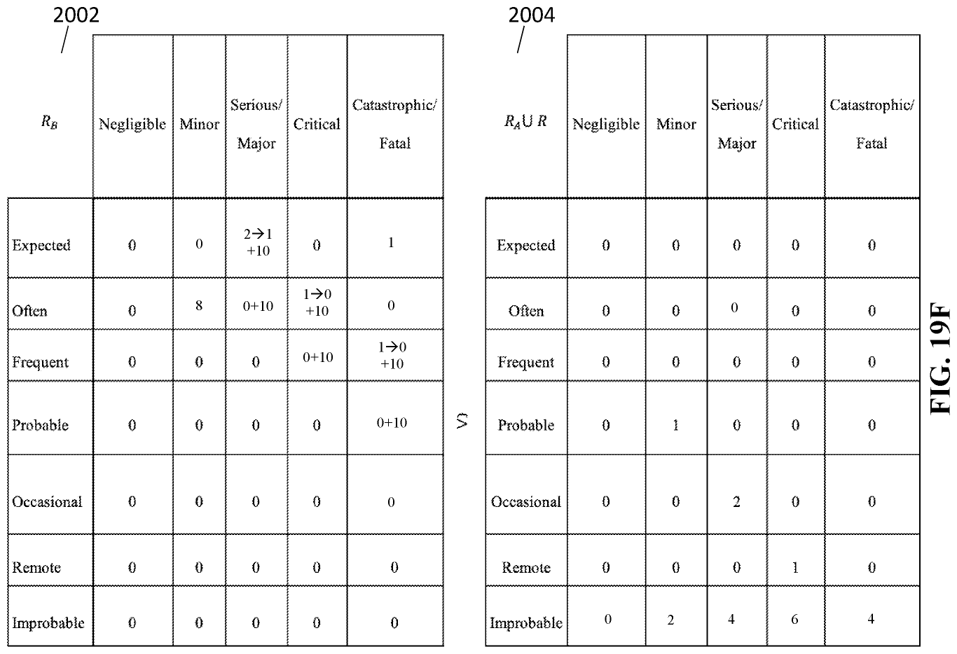

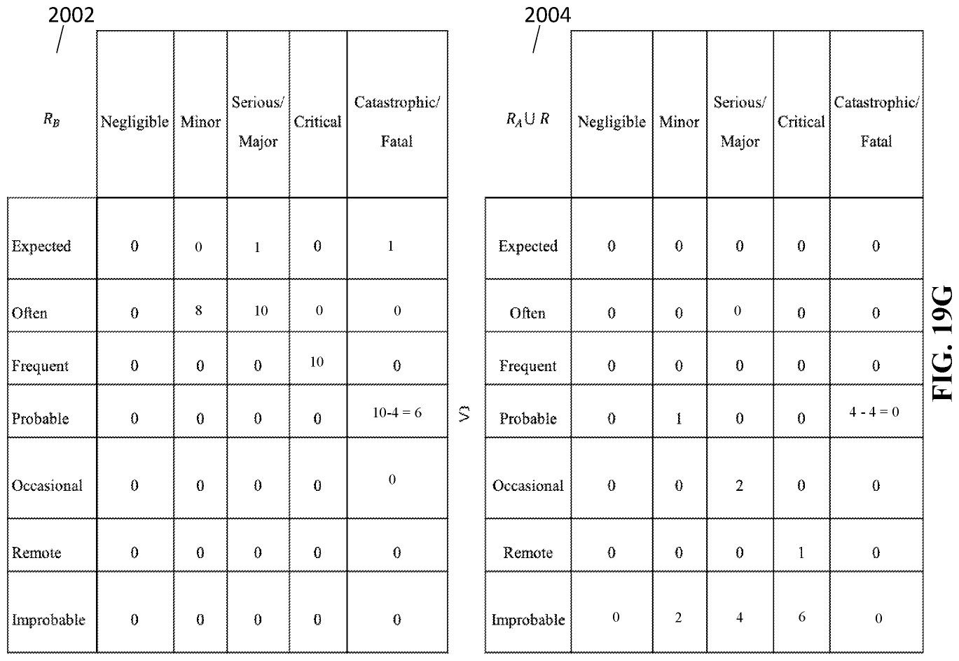

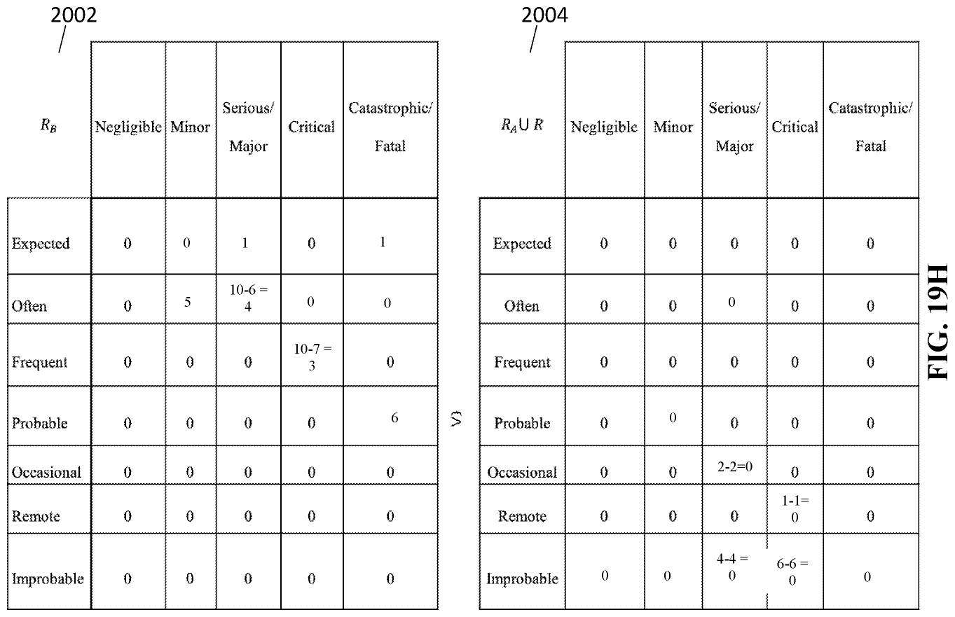

Further, Equation 9 can be populated using R_{B} (e.g., per Table 6) and R_{A} ∪R, producing Equation 25, shown in FIG. 19A. Specifically, tables 2002 and 2004 are shown for R_{B} and R_{A} ∪R, respectively.

Next, with particular reference to block 1512, Equation 9 can be simplified to determine whether benefit exceeds risk. For instance, the “Removing Identical Risks” rule can be used to simplify Equation 25. The cell at the junction “Often” and “Negligible” (cell 2006) has a value of “1” on the left-hand side, while the corresponding cell (cell 2008) on the right-hand side also has a value of “1.” By applying the “Removing Identical Risks” rule to both sides of the equation, this value can be removed (i.e., by subtracting “1”) without changing which side of the equation has the most risk. The result is shown in Equation 26, shown in FIG. 19B.

This equation can be further simplified using, for instance, the “Moving Redundant Risks” and “Removing Identical Risks” rules. For example, the left-hand side 2002 of the equation has a count of “1” the “Expected/Minor” cell (cell 2010). Using the “Moving Redundant Risks” rule, the count of “1” in cell 2010 can be removed and a count of “10” can be added to the “Often/Minor” cell (cell 2016), without changing which side of the equation has the most risk. This result is shown in Equation 27 in FIG. 19C.

Now, the “Moving Similar Risks” rule can be used to simplify Equation 27 to Equation 28, shown in FIG. 19D. As can be seen, one risk can be moved from the left-hand side's “Often/Minor” cell to the “Expected/Negligible” cell, and another risk can be moved to the “Frequent/Serious/Major” cell without changing which side of the equation has the most risk.

Equation 28 can be further simplified. There is an “Expected/Negligible” and a “Frequent/Serious/Major” risk on both the left-hand side 2002 and the right-hand side 2004 of the equation. Therefore, by applying the “Removing Identical Risks” rule, an “Expected/Negligible” risk and a “Frequent/Serious/Major” risk can be removed from each side of the equation without changing which side of the equation has the most risk. This is shown in Equation 29 in FIG. 19E.

FIG. 19E shows a pattern that is useful for simplifying the remainder of the equation. Both sides of the equation have no risks in the “Negligible” column. For all of the other columns, the left-hand side 2002 has at least one risk in each column and in the three most-frequent rows (that is, “Expected,” “Often,” and “Frequent”), and the right-hand side 2004 has much larger numbers of risks in each column and in the least-frequent four rows (that is, “Probable,” “Occasional,” “Remote,” and “Improbable”). This itself indicates that the risk total in the left-hand side is greater than the risk total in the right-hand side and, therefore, the benefits of giving the patient population a transfusion exceeds the risk of giving the patient population a transfusion.

However, further simplification can be performed. By using the “Moving Redundant Risks” rule on each column of the left-hand side, there will be enough risks in each column of the left-hand side of the equation to cancel all of the risks in the same column on the right-hand side of the equation. First, on the left-hand side of the equation, the “Moving Repeated Risks” rule once for each of the three right-most columns (that is, “Serious/Major,” “Critical,” and “Catastrophic/Fatal”) yields Equation 30, shown in FIG. 19F. Second, if the “Remove Unequal Risks” rule is used on the “Catastrophic/Fatal” column on Equation 30, then Equation 31 (shown in FIG. 19G) is generated. Now, the left-most and right-most columns of the right-hand side 2004 of the equation have no risks. Third, if the “Remove Unequal Risks” rule is used on the “Critical” and “Serious/Major” columns of Equation 31, then Equation 32 (shown in FIG. 19H) is generated. This equation now has no risks left on the right-hand side and 19 risks on the left-hand side. Therefore, there are more risks on the left-hand side and the equation is true. Accordingly, for this patient population, the benefit of a blood transfusion outweighs the risk.

Finally, with particular reference to block 1514 in FIG. 14, the above benefit-risk decision and/or rationales supporting each of the aforementioned steps and/or simplifications can be documented and/or outputted. Such output may be, for instance, via a GUI, a summary document, or the like.

### Example 2

In this example, with particular reference to block 1502, the healthcare action is a blood transfusion and the applicable patient population is patients in the United States who have upper gastrointestinal (GI) bleeding. Unlike in Example 1, this patient population may, or may not, meet ER criteria for needing a blood transfusion.

The purpose and risks of performing such blood transfusions to these patients may be identified by reference to knowledge of ER medicine, physicians specializing in ER medicine, knowledge of medical devices (e.g., apheresis equipment) for collecting and infusing blood, and the like.

With particular reference to block 1506, the risks in Table_{R}_{_{B}}, Table_{R}_{_{A}}, and Table_{R} can be identified. Table_{R}_{_{B}} can be generated by identifying the health conditions that might cause this patient population to receive a blood transfusion (e.g., health conditions that might cause a bleeding ulcer to lose enough blood that a transfusion might be indicated to treat the blood loss). Such a table would be similar to Table 11, shown in Example 1 (e.g., describing the conditions of “altered mental state,” “circulatory collapse,” “heart attack, stroke, kidney injury,” and “esophageal varices”).

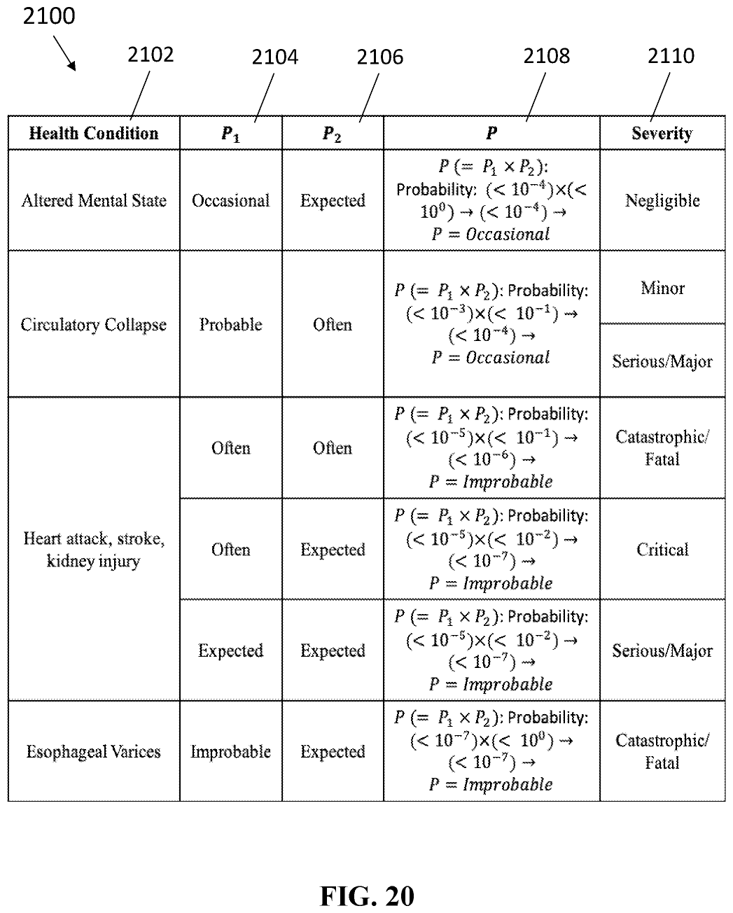

Based on the specific health condition, probability and severity categories can be identified and assigned, as shown in FIG. 20. This figure is similar to FIG. 15, set forth above herein with respect to Example 1. FIG. 20 shows table 2100, which has five columns, columns 2102, 2104, 2106, 2108, and 2110. Column 2102 shows the various health conditions, specifically, in this example, altered mental state; circulatory collapse; heart attack, stroke, and kidney injury; and esophageal varices. Column 2104 shows P₁, or probability, for each of these conditions. Column 2106 shows P₂, or probability and severity, for each of these conditions. Column 2108 shows P for each of the listed conditions, with P (=P₁×P₂). Column 1610 shows the severity for each of the listed conditions.

P₁ can be obtained from various sources of information, including, for instance, reference to emergency room medicine resources and/or physicians. For example, hemorrhagic shock may be relevant to the altered mental state condition. Such shock can be defined as, for instance, (1) volume loss from 30% to 40% of total blood volume, from 1500 mL to 2000 mL, (2) a significant drop in blood pressure and changes in mental status, (3) heart rate and respiratory rate elevation (e.g., more than 120 beats per minute (BPM)), (4) urine output decline, and/or (5) capillary refill delays. With respect to circulatory collapse, studies have indicated around a 5% incidence. With respect to esophageal varices, between 1966 and 2013, there have been about 50 cases of esophageal varices triggered by transfusions. According to the Red Cross, there are 29,000 transfusions per day, or 10,585,000 transfusions per year.

Similarly, P₂ can be obtained from various sources of information, including, for instance, reference to emergency room medicine resources and/or physicians.

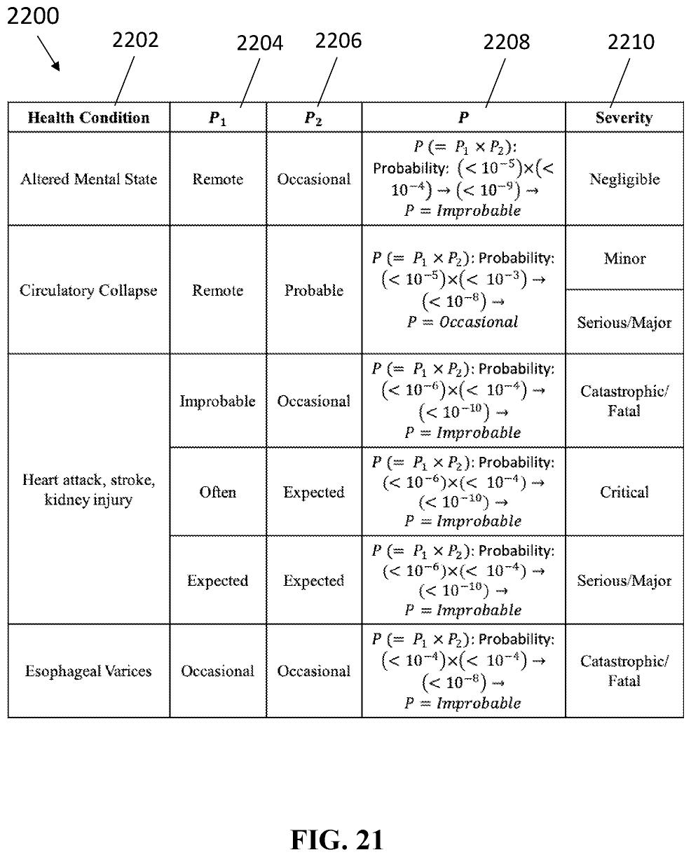

Next, with particular reference to block 1506, each cell in the following table for R_{B} can be populated. To do so, note the severity and the probability from FIG. 21, and enter the number of risks with that cell's combination of severity and probability.

TABLE 16
Sample Table for R_{B}

| | | | Serious/ | | Catastrophic/ |
|---|---|---|---|---|---|
| R_{B} | Negligible | Minor | Major | Critical | Fatal |
| Expected | 0 | 0 | 0 | 0 | 0 |
| Often | 0 | 0 | 0 | 0 | 0 |
| Frequent | 0 | 0 | 0 | 0 | 0 |
| Probable | 0 | 0 | 0 | 0 | 0 |
| Occasional | 1 | 1 | 1 | 0 | 0 |
| Remote | 0 | 0 | 0 | 0 | 0 |
| Improbable | 0 | 0 | 1 | 1 | 2 |

Table_{R}_{_{A}} can be generated by identifying the health conditions in this patient population that might remain after receiving the healthcare action. Since B=R_{B}−R_{A}, Table_{R}, will normally contain the same health conditions as Table_{R}_{_{B}}, particularly since Table_{R} contains any health risks that were created by the healthcare action.

FIG. 21 shows a table 2200 for R_{A}. As with FIG. 20, table 2200 contains column 2202 for the various health conditions, column 2204 showing P₁, or probability, for each of these conditions, column 2206 showing P₂, or probability and severity, for each of these conditions, column 2208 showing P for each of the listed conditions, with P (=P₁×P₂), and column 2210 showing the severity for each of the listed conditions.

Next, with particular reference to block 1506, each cell in the following table for R_{A} can be populated. To do so, note the severity and the probability from FIG. 21, and enter the number of risks in the previous table with that cell's combination of severity and probability.

TABLE 17
Sample Table for R_{A}

| | | | Serious/ | | Catastrophic/ |
|---|---|---|---|---|---|
| R_{A} | Negligible | Minor | Major | Critical | Fatal |
| Expected | 0 | 0 | 0 | 0 | 0 |
| Often | 0 | 0 | 0 | 0 | 0 |
| Frequent | 0 | 0 | 0 | 0 | 0 |
| Probable | 0 | 0 | 0 | 0 | 0 |
| Occasional | 0 | 0 | 0 | 0 | 0 |
| Remote | 0 | 0 | 0 | 0 | 0 |
| Improbable | 1 | 1 | 2 | 1 | 2 |

Next, R can be populated by identifying the health conditions that, for this patient population, might remain after the blood transfusion. These conditions are the same as that shown in Table 14 with respect to Example 1.

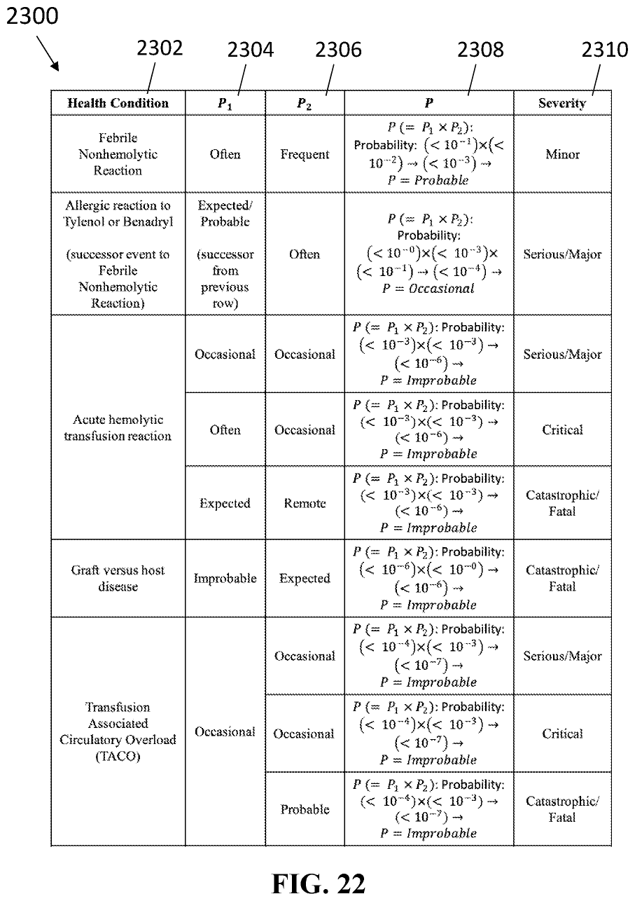

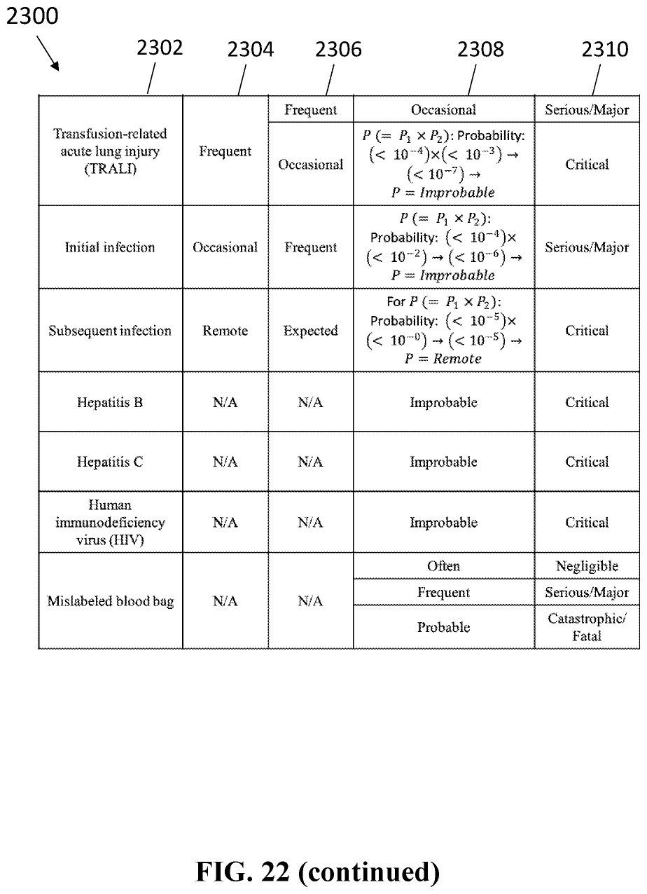

Based on the above health condition, probability and severity categories can be identified and assigned, as shown in FIG. 22. This figure is therefore similar to FIG. 17, shown above herein with respect to Example 1. FIG. 22 shows table 2300 with five columns, columns 2302, 2304, 2306, 2308, and 2310. Column 2302 shows the various health conditions, specifically, in this example, febrile non-hemolytic reaction, allergic reaction subsequent to the febrile non-hemolytic reaction, acute hemolytic transfusion reaction, graft versus host disease, transfusion associated circulatory overload (TACO), transfusion-related acute lung injury (TRALI), initial infection, subsequent infection, hepatitis B, hepatitis C, human immunodeficiency virus (HIV), and a mislabeled blood bag. Column 2304 shows P₁, or probability, for each of these conditions. Column 2306 shows P₂, or probability and severity, for each of these conditions. Column 2308 shows P for each of the listed conditions, with P (=P₁×P₂). Column 2310 shows the severity for each of the listed conditions.

P₁ can be obtained from various sources of information, including, for instance, reference to emergency room medicine resources and/or physicians. Various exemplar statistics relating to the listed health conditions has been set forth above herein with respect to the description of FIG. 17.

Similarly, P₂ can be obtained from various sources of information, including, for instance, reference to emergency room medicine resources and/or physicians.

Next, with particular reference to block 1506, each cell in the following table for R can be populated. To do so, note the severity and the probability from FIG. 22, and enter the number of risks with that cell's combination of severity and probability.

TABLE 18
Sample Table for R

| | | | Serious/ | | Catastrophic/ |
|---|---|---|---|---|---|
| R | Negligible | Minor | Major | Critical | Fatal |
| Expected | 0 | 0 | 0 | 0 | 0 |
| Often | 1 | 0 | 0 | 0 | 0 |
| Frequent | 0 | 0 | 1 | 0 | 0 |
| Probable | 0 | 1 | 0 | 0 | 1 |
| Occasional | 0 | 0 | 2 | 0 | 0 |
| Remote | 0 | 0 | 0 | 1 | 0 |
| Improbable | 0 | 0 | 3 | 6 | 3 |

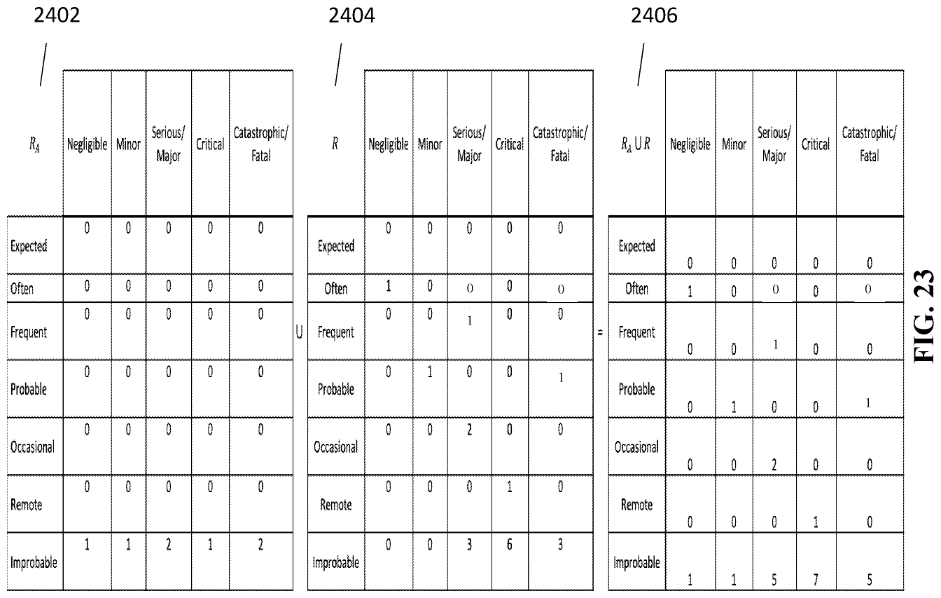

Then, with particular reference to blocks 1508 and 1510, R_{A} ∪R can be calculated and Equation 9 can be populated accordingly. Specifically, R_{A} ∪R can be calculated using R_{A} and R, and their respective tables above. This generates Equation 35, shown in FIG. 23. Specifically, tables 2402, 2404, and 2406 are shown for R_{A}, R, and R_{A} ∪R, respectively.

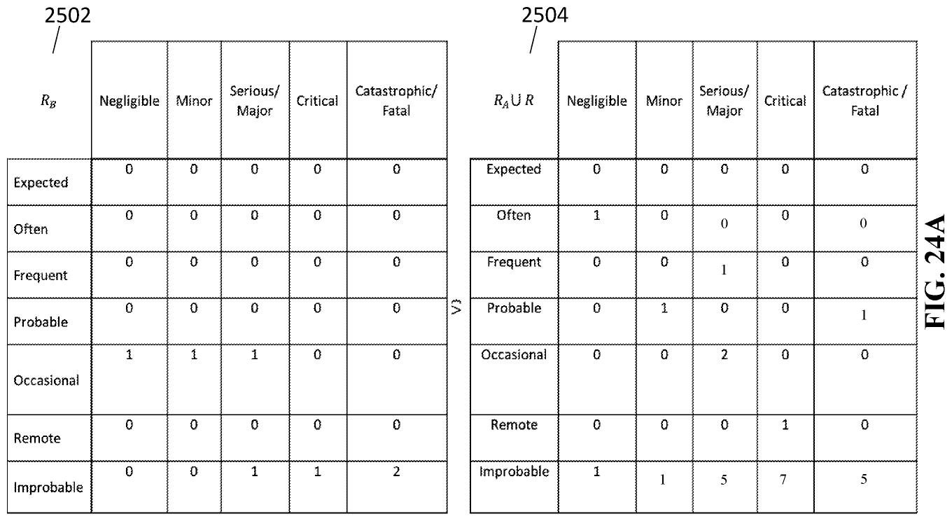

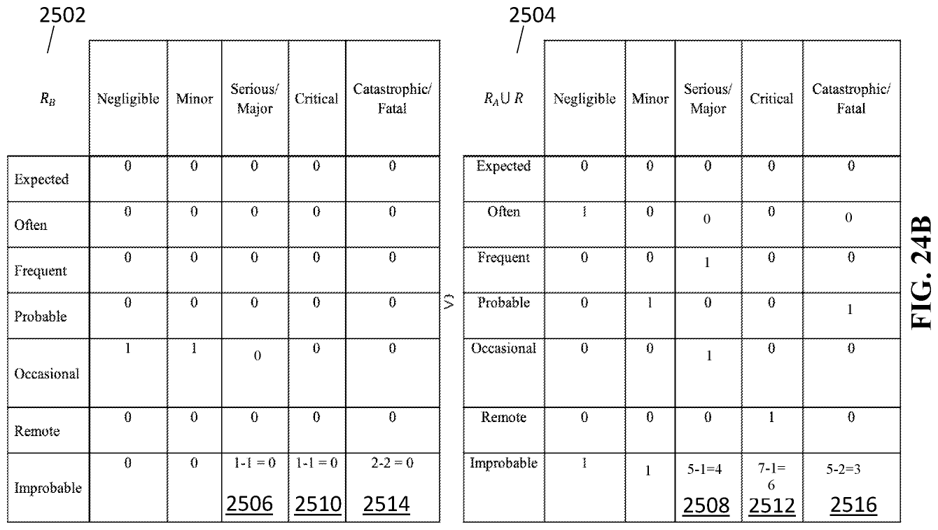

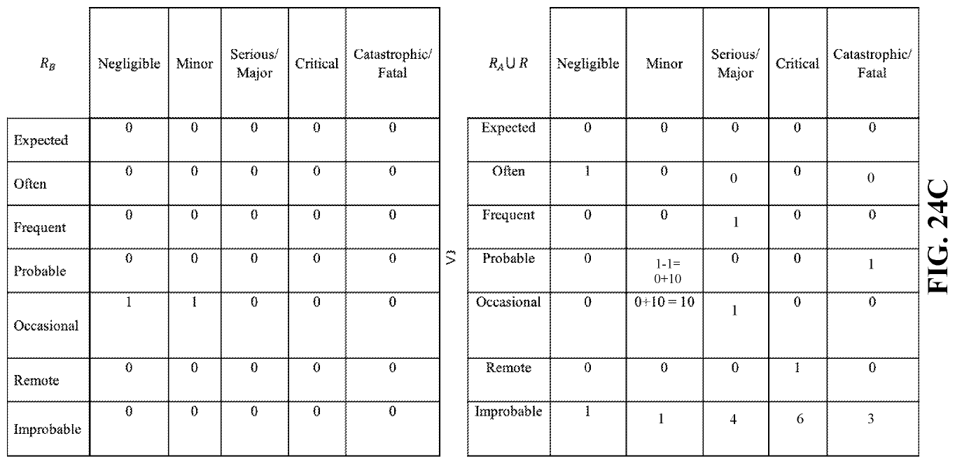

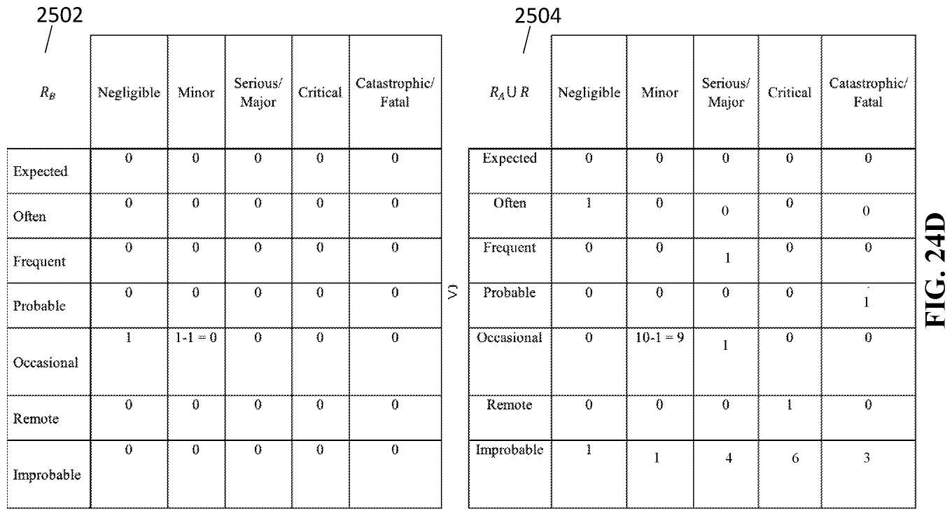

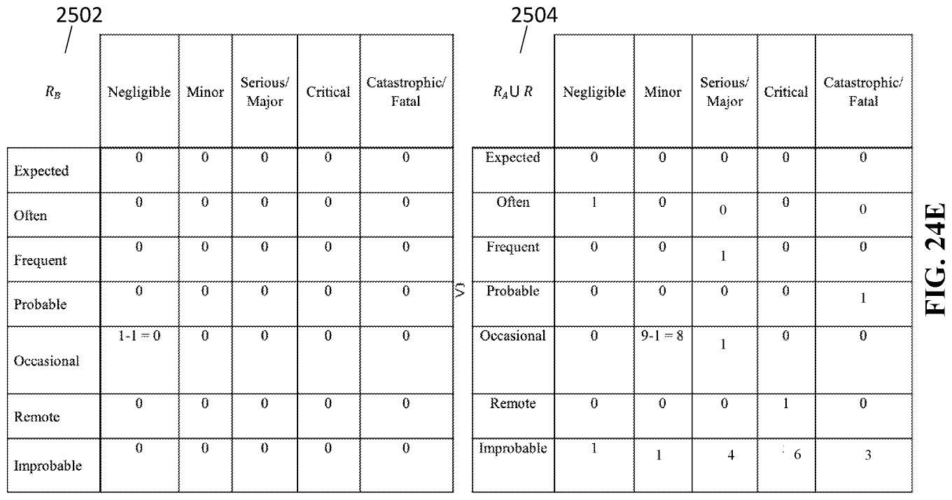

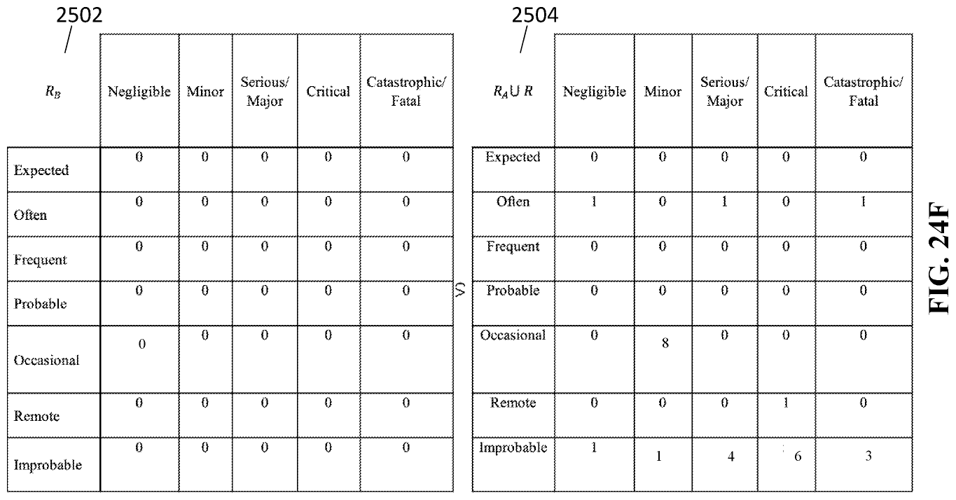

Further, Equation 9 can be populated using R_{B} (e.g., per Table 6) and R_{A} ∪R, producing Equation 36, shown in FIG. 24A. Specifically, tables 2502 and 2504 are shown for R_{B} and R_{A} ∪R, respectively.

Next, with particular reference to block 1512, Equation 9 can be simplified to determine whether benefit exceeds risk. For instance, the “Removing Identical Risks” rule can be used to simplify Equation 36 to produce Equation 37, shown in FIG. 24B. First, a count of “1” can be removed from cell 2506 on the left-hand side 2502 of the equation, and a corresponding count of “1” can also be removed from cell 2508 on the right-hand side 2504 of the equation. Second, a count of “1” can be removed from cell 2510 on the left-hand side 2502 and from the corresponding cell 2512 on the right-hand side 2504. Third, a count of “2” can be removed from cell 2514 on the left-hand side 2502 and from the corresponding cell 2516 on the right-hand side 2504.

This equation can be further simplified using, for instance, the “Moving Redundant Risk” rule, which produces Equation 38, shown in FIG. 24C.

The “Removing Identical Risks” rule can be used to further simplify Equation 38, producing Equation 39, shown in FIG. 24D.

Further, the “Removing Unequal Risks” rule can be used within the “Minor,” “Serious/Major,” “Critical,” and “Catastrophic/Fatal” columns to further simplify Equation 39, producing Equation 40, shown in FIG. 24E. This equation then simplifies to Equation 41, shown in FIG. 24F.

It is apparent from the values shown in FIG. 24F that the risk total on the right-hand side is greater than the risk total on the left-hand side, especially since there are no risks remaining in any of the cells on the left-hand side. This means that the risks of giving this patient population a transfusion outweighs the benefits of doing so.

Because the amount of risk on the right-hand side of FIG. 24F is such a large fraction of the initial risk in FIG. 24A, there is nothing to gain by following the process in FIG. 5. However, in cases where the amount of risk on the left and right hand sides of the equation are much closer, the additional processes in FIG. 5 can be followed to confirm that the risk exceeds the benefit.

Finally, with particular reference to block 1514 in FIG. 14, the above benefit-risk decision and/or rationales supporting each of the aforementioned steps and/or simplifications can be documented and/or outputted. Such output may be, for instance, via a GUI, a summary document, or the like.

Accordingly, embodiments of the present disclosure enable the determination of whether the benefits of a product and/or service exceeds the risk. In at least one example, methods are disclosed to determine if the benefit of a medical device and/or medical procedure exceeds the risk.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised.

The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

What is claimed is:

1. A method for analyzing one or more healthcare decisions, the method comprising:
defining, by at least one processor, one or more healthcare decisions to be made;
for each of the defined one or more healthcare decisions, performing a benefit-risk analysis by:
defining, by the at least one processor, a patient population to which a healthcare action is to be applied,
identifying, by the at least one processor, one or more health conditions that would cause the healthcare action to be applied to the patient population,
defining, by the at least one processor, variables R_{B}, R_{A}, and R, wherein:
R_{B} is an amount of risk from a health condition that affects at least one portion of the patient population before the healthcare action is applied,
R_{A} is an amount of risk from the health condition of the at least one portion of the patient population after the healthcare action is applied,
R is an amount of risk from applying the healthcare action to the at least one portion of the patient population,
defining, by the at least one processor, an equation in which R_{B} [symbol] R_{A}∪R,
determining, by the at least one processor, R_{B} and R_{A}∪R,
determining from the equation, by the at least one processor, whether (i) the amount of risk from the health condition that affects the at least one portion of the patient population before the healthcare action is applied is greater than a combination of (ii) the amount of risk from the health condition of the at least one portion of the patient population after the healthcare action is applied, and (iii) the amount of risk from applying the healthcare action to the at least one portion of the patient population, and
documenting, by the at least one processor, the benefit-risk analysis;
transmitting, by the at least one processor, a first alert to at least one computing device, the first alert containing an answer for each of the one or more healthcare decisions;
transmitting, by the at least one processor, a second alert to the at least one computing device when the equation comprises an error,
wherein the first alert and the second alert are both sent in real-time or near real-time,
wherein the healthcare action comprises a medical device.

2. The method of claim 1, wherein each of R_{B}, R_{A}, and R is a set of estimates of individual risks, and wherein an overall risk is represented by a totality of the individual risks in a given set of estimates.

3. The method of claim 2, wherein each estimate in each set of estimates is an ordered pair whose ordinate is a text description of a level of probability and whose abscissa is a text description of a level of severity.

4. The method of claim 1, wherein the calculating further comprises:
generating, by the at least one processor, a table for R_{B} as Table_{R}_{_{B}},
generating, by the at least one processor, a table for R_{A} as Table_{R}_{_{A}},
generating, by the at least one processor, a table for R as Table_{R},
wherein:
Table_{R}_{_{B}} comprises a matrix of probability rankings and severity rankings for R_{B},
Table_{R}_{_{A}} comprises a matrix of probability rankings and severity rankings for R_{A}, and
Table_{R} comprises a matrix of probability rankings and severity rankings for R.

5. The method of claim 4, wherein a range of all possible risks is divided into at least 35 categories, wherein the at least 35 categories comprise at least seven probability rankings and at least five severity rankings, and wherein each cell of Table_{R}_{_{B}}, Table_{R}_{_{A}}, and Table_{R} contains a count of a number of occurrences of a combination of probability and severity for a respective row and column.

6. The method of claim 4, wherein the probability rankings in each of Table_{R}_{_{B}}, Table_{R}_{_{A}}, and Table_{R} range from less than 0.00001% to 100%.

7. The method of claim 4, wherein the severity rankings in each of Table_{R}_{_{B}}, Table_{R}_{_{A}}, and Table_{R} are qualitative and/or descriptive of potential patient outcomes.

8. The method of claim 1, wherein the determining from the equation further comprises:
simplifying, by the at least one processor, the equation using at least one rule in a plurality of rules,
wherein the plurality of rules comprises:
a first rule in which identical risks in the equation are removed from the equation,
a second rule in which unequal risks in the equation are removed from the equation,
a third rule in which representations of repeated risks are changed, to move locations of the repeated risks, and
a fourth rule in which representations of similar risks are changed, to move locations of the similar risks.

9. The method of claim 1, further comprising:
defining, by the at least one processor, additional benefit-risk decisions to be made with respect to one or more alternatives to the healthcare action; and
repeating, by the at least one processor, the benefit-risk analysis for each of the additional benefit-risk decisions.

10. The method of claim 1, further comprising at least one of:
adding, by the at least one processor, a time component to any risk associated with the healthcare action, and
adding, by the at least one processor, a separate risk associated with the healthcare action that varies with time.

11. The method of claim 1, further comprising:
for a patient in the patient population, modeling, by the at least one processor, a benefit that does not begin until after a period of time has passed and then continues, unchanged, for a remainder of the patient's life,
wherein the modeling comprises:
defining a variable L as the remainder of the patient's life,
defining a variable Tᵢ as the period of time,
weighting the benefit over time based on how long the patient is expected to experience the benefit,
defining, for each i^{th} risk, equations Wᵢ=L−Tᵢ and Rᵢ=(Pᵢ, Sᵢ, L−Tᵢ),
modifying Wᵢ to reflect a deferred value of the benefit not beginning immediately, and
defining an equation where R={(P₁, S₁, L−T₁), (P₂, S₂, L−T₂), (P₃, S₃, L−T₃), . . . , (Pₙ, Sₙ, L−Tₙ)}.

12. A method for analyzing one or more healthcare decisions, the method comprising:
defining, by at least one processor, one or more healthcare decisions to be made with respect to a healthcare action;
for each of the defined one or more healthcare decisions, performing a benefit-risk analysis by:
defining, by the at least one processor, a patient population to which the healthcare action is to be applied,
constructing, by the at least one processor, a first table listing a first set of health conditions that would cause the healthcare action to be applied to the patient population,
constructing, by the at least one processor, a second table listing (i) a probability for each condition in the first set of health conditions existing before the healthcare action is applied to the patient population, (ii) a severity ranking for each condition in the first set of health conditions existing before the healthcare action is applied to the patient population, and (iii) a combination of probability and severity for each condition in the first set of health conditions existing before the healthcare action is applied to the patient population,
generating, by the at least one processor, a Table_{R}_{_{B}} that comprises a matrix of probability rankings and severity rankings based on the second table,
constructing, by the at least one processor, a third table listing (i) a probability for each condition in the first set of health conditions existing after the healthcare action is applied to the patient population, (ii) a severity ranking for each condition in the first set of health conditions existing after the healthcare action is applied to the patient population, and (iii) a combination of probability and severity for each condition in the first set of health conditions existing after the healthcare action is applied to the patient population,
generating, by the at least one processor, a Table_{R}_{_{A}} that comprises a matrix of probability rankings and severity rankings based on the third table,
constructing, by the at least one processor, a fourth table listing a second set of health conditions that could result from application of the healthcare action to the patient population,
constructing, by the at least one processor, a fifth table listing (i) a probability for each condition in the second set of health conditions existing because of the application of the healthcare action to the patient population, (ii) a severity ranking for each condition in the second set of health conditions existing because of the application of the healthcare action to the patient population, and (iii) a combination of probability and severity for each condition in the second set of health conditions existing because of the application of the healthcare action to the patient population,
generating, by the at least one processor, a Table_{R} that comprises a matrix of probability rankings and severity rankings based on the fifth table,
determining, by the at least one processor, R_{A} ∪R based on the Table_{R}_{_{A}} and the Table_{R},
constructing, by the at least one processor, a Table_{R}_{_{A}}_{∪R},
generating, by the at least one processor, an equation in which Table_{R}_{_{B}} [symbol] Table_{R}_{_{A}}_{∪R},
simplifying, by the at least one processor, the equation using at least one of a plurality of simplification rules, the plurality of simplification rules comprising:
a first rule in which identical risks on both sides of the equation are removed,
a second rule in which repeated risks on one side of the equation are represented differently,
a third rule in which similar risks on one side of the equation are represented differently, and
a fourth rule in which unequal risks on both sides of the equation are removed,
reviewing, by the at least one processor, the simplified equation to determine whether (i) an amount of risk that exists before application of the healthcare action is greater than a combination of (ii) an amount of risk from the application of the healthcare action, and (iii) an amount of risk that exists after the application of the healthcare action, and
transmitting, by the at least one processor, a plurality of alerts to at least one computing device, the plurality of alerts containing an answer for each of the one or more healthcare decisions and a notification when an error occurs with at least one of: (i) the constructing the first table, (ii) the constructing the second table, (iii) the constructing the third table, (iv) the constructing the fourth table, (v) the constructing the fifth table, (vi) the determining R_{A} ∪R, (vii) the constructing the Table_{R}_{_{A}}_{∪R}, and (viii) the simplifying the equation,
wherein the healthcare action comprises a medical device.

13. The method of claim 12, wherein the probability rankings for each of the Table_{R}_{_{B}}, Table_{R}_{_{A}}, and Table_{R} comprise seven semi-quantitative probability levels.

14. The method of claim 13, wherein the seven semi-quantitative probability levels range from less than 0.00001% to 100%.

15. The method of claim 14, wherein the severity rankings for each of the Table_{R}_{_{B}}, Table_{R}_{_{A}}, and Table_{R} comprise five qualitative probability levels.

16. The method of claim 12, further comprising:
quantifying, by the at least one processor, one or more uncertainty ranges in the equation, and
modifying, by the at least one processor, the benefit-risk analysis based on the one or more uncertainty ranges.

17. The method of claim 12, wherein the healthcare action is purely aesthetic such that the healthcare action only benefits mental and/or emotional states of patients in the patient population.

18. A non-transitory computer-readable storage medium, having instructions stored thereon that, when executed by a computing device, cause the computing device to perform operations, the operations comprising:
defining one or more healthcare decisions to be made with respect to a healthcare action;
for each of the defined one or more healthcare decisions, performing a benefit-risk analysis by:
defining a patient population to which the healthcare action is to be applied,
identifying one or more health conditions that would cause the healthcare action to be applied to the patient population,
defining variables R_{B}, R_{A}, and R, wherein:
R_{B} is an amount of risk from a health condition that affects at least one portion of the patient population before the healthcare action is applied,
R_{A} is an amount of risk from the health condition that affects the at least one portion of the patient population after the healthcare action is applied,
R is an amount of risk from applying the healthcare action to the at least one portion of the patient population,
defining an equation in which R_{B} [symbol] R_{A}∪R,
determining R_{B} and R_{A}∪R,
determining the equation to determine whether (i) the amount of risk from the health condition that affects the at least one portion of the patient population before the healthcare action is applied is greater than a combination of (ii) the amount of risk from the health condition that affects the at least one portion of the patient population after the healthcare action is applied, and (iii) the amount of risk from applying the healthcare action to the at least one portion of the patient population, and
determining the benefit-risk analysis;
transmitting an alert to at least one computing device, the alert containing an answer for each of the defined one or more healthcare decisions;
transmitting a notification to the at least one computing device when an error occurs with the determining R_{B} and R_{A}∪R and the determining the equation,
wherein the healthcare action comprises a medical device.

19. The non-transitory computer-readable storage medium of claim 18, wherein the operations further comprise:
displaying, by a graphical user interface (GUI), the alert and the notification.

20. The non-transitory computer-readable storage medium of claim 19, wherein the calculating further comprises:
generating a table for R_{B} as Table_{R}_{_{B}},
generating a table for R_{A} as Table_{R}_{_{A}}, and
generating a table for R as Table_{R}.

21. The non-transitory computer-readable storage medium of claim 20, wherein:
Table_{R}_{_{B}} comprises a matrix of semi-quantitative probability rankings and qualitative severity rankings for R_{B},
Table_{R}_{_{A}} comprises a matrix of semi-quantitative probability rankings and qualitative severity rankings for R_{A}, and
Table_{R} comprises a matrix of semi-quantitative probability rankings and qualitative severity rankings for R.

22. The non-transitory computer-readable storage medium of claim 21, wherein the determining the equation further comprises:
simplifying the equation using at least one rule in a plurality of rules,
wherein the plurality of rules comprises:
a first rule in which identical risks in the equation are removed,
a second rule in which unequal risks in the equation are removed,
a third rule in which repeated risks in the equation are moved, and
a fourth rule in which similar risks in the equation are moved.

23. The non-transitory computer-readable storage medium of claim 22, wherein the operations further comprise:
quantifying one or more uncertainty ranges in the equation, and
modifying the benefit-risk analysis based on the one or more uncertainty ranges.
